SEQUENCE LISTING (226 sequences)

SEQ ID NO: 1 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 2 - protein, 5 aa, Artificial Sequence
  source: /note=“Description of Artificial Sequence Synthetic peptide”
DGGGS

SEQ ID NO: 3 - protein, 5 aa, Artificial Sequence
  source: /note=“Description of Artificial Sequence Synthetic peptide”
TGEKP

SEQ ID NO: 4 - protein, 9 aa, Artificial Sequence
  source: /note=“Description of Artificial Sequence Synthetic peptide”
LRQKDGERP

SEQ ID NO: 5 - protein, 4 aa, Artificial Sequence
  source: /note=“Description of Artificial Sequence Synthetic peptide”
GGRR

SEQ ID NO: 6 - protein, 8 aa, Artificial Sequence
  source: /note=“Description of Artificial Sequence Synthetic peptide”
GGRRGGGS

SEQ ID NO: 7 - protein, 9 aa, Artificial Sequence
  source: /note=“Description of Artificial Sequence Synthetic peptide”
LRQRDGERP

SEQ ID NO: 8 - protein, 12 aa, Artificial Sequence
  source: /note=“Description of Artificial Sequence Synthetic peptide”
LRQKDGGGSERP

SEQ ID NO: 9 - protein, 16 aa, Artificial Sequence
  source: /note=“Description of Artificial Sequence Synthetic peptide”
LRQKDGGGSGGGSERP

SEQ ID NO: 10 - protein, 6 aa, Artificial Sequence
  source: /note=“Description of Artificial Sequence Synthetic peptide”
TGSQKP

SEQ ID NO: 11 - protein, 5 aa, Artificial Sequence
  source: /note=“Description of Artificial Sequence Synthetic peptide”
GGGGS

SEQ ID NO: 12 - protein, 96 aa, Homo sapiens
DAKSLTAWSRTLVTFKDVFVDFTREEWKLLDTAQQIVYRNVMLENYKNLVSLGYQLTKPDVILRLEKGEEPWLVEREIHQETHPDSETAFEIKSSV

SEQ ID NO: 13 - DNA, 24 bp, Artificial Sequence
  source: /note=“Description of Artificial Sequence Synthetic primer”
gcagagctctctggctaactagag

SEQ ID NO: 14 - DNA, 200 bp, Artificial Sequence
  source: /note=“Description of Artificial Sequence Synthetic primer”
ttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttctggcaactagaaggcacag

SEQ ID NO: 15 - protein, 7 aa, Artificial Sequence
  source: /note=“Description of Artificial Sequence Synthetic peptide”
TGEKPFA

SEQ ID NO: 16 - protein, 8 aa, Artificial Sequence
  source: /note=“Description of Artificial Sequence Synthetic peptide”
TGSQKPFQ

SEQ ID NO: 17 - protein, 12 aa, Artificial Sequence
  source: /note=“Description of Artificial Sequence Synthetic peptide”
LRQKDAARGSGG

SEQ ID NO: 18 - protein, 6 aa, Artificial Sequence
  source: /note=“Description of Artificial Sequence Synthetic peptide”
LRGSGG

SEQ ID NO: 19 - DNA, 21 bp, Homo sapiens
atccgagatagggacgaggag

SEQ ID NO: 20 - protein, 7 aa, Artificial Sequence
  source: /note=“Description of Artificial Sequence Synthetic peptide”
QSGHLAR

SEQ ID NO: 21 - protein, 7 aa, Artificial Sequence
  source: /note=“Description of Artificial Sequence Synthetic peptide”
QRKDLTQ

SEQ ID NO: 22 - protein, 7 aa, Artificial Sequence
  source: /note=“Description of Artificial Sequence Synthetic peptide”
RSDHLSR

SEQ ID NO: 23 - protein, 7 aa, Artificial Sequence
  source: /note=“Description of Artificial Sequence Synthetic peptide”
TRDGLRQ

SEQ ID NO: 24 - protein, 7 aa, Artificial Sequence
  source: /note=“Description of Artificial Sequence Synthetic peptide”
RSANLAR

SEQ ID NO: 25 - protein, 7 aa, Artificial Sequence
  source: /note=“Description of Artificial Sequence Synthetic peptide”
DSSDRKK

SEQ ID NO: 26 - DNA, 20 bp, Homo sapiens
ccgagatagggacgaggagc

SEQ ID NO: 27 - protein, 7 aa, Artificial Sequence
  source: /note=“Description of Artificial Sequence Synthetic peptide”
RSDNLST

SEQ ID NO: 28 - protein, 7 aa, Artificial Sequence
  source: /note=“Description of Artificial Sequence Synthetic peptide”
RSDNLTR

SEQ ID NO: 29 - protein, 7 aa, Artificial Sequence
  source: /note=“Description of Artificial Sequence Synthetic peptide”
DRSDLSR

SEQ ID NO: 30 - protein, 7 aa, Artificial Sequence
  source: /note=“Description of Artificial Sequence Synthetic peptide”
RREHLRA

SEQ ID NO: 31 - protein, 7 aa, Artificial Sequence
  source: /note=“Description of Artificial Sequence Synthetic peptide”
TSGNLTR

SEQ ID NO: 32 - protein, 7 aa, Artificial Sequence
  source: /note=“Description of Artificial Sequence Synthetic peptide”
QRSHLSD

SEQ ID NO: 33 - DNA, 21 bp, Homo sapiens
cagggaaagcagcgagcgccg

SEQ ID NO: 34 - protein, 7 aa, Artificial Sequence
  source: /note=“Description of Artificial Sequence Synthetic peptide”
ERGTLAR

SEQ ID NO: 35 - protein, 7 aa, Artificial Sequence
  source: /note=“Description of Artificial Sequence Synthetic peptide”
MACCRYA

SEQ ID NO: 36 - protein, 7 aa, Artificial Sequence
  source: /note=“Description of Artificial Sequence Synthetic peptide”
RSADLTR

SEQ ID NO: 37 - protein, 7 aa, Artificial Sequence
  source: /note=“Description of Artificial Sequence Synthetic peptide”
QSGDLTR

SEQ ID NO: 38 - protein, 7 aa, Artificial Sequence
  source: /note=“Description of Artificial Sequence Synthetic peptide”
QSGNLAR

SEQ ID NO: 39 - protein, 7 aa, Artificial Sequence
  source: /note=“Description of Artificial Sequence Synthetic peptide”
RQEHRVA

SEQ ID NO: 40 - DNA, 20 bp, Homo sapiens
gaaagcagcgagcgccggga

SEQ ID NO: 41 - protein, 7 aa, Artificial Sequence
  source: /note=“Description of Artificial Sequence Synthetic peptide”
RSAHLSR

SEQ ID NO: 42 - protein, 7 aa, Artificial Sequence
  source: /note=“Description of Artificial Sequence Synthetic peptide”
MSCCRSA

SEQ ID NO: 43 - protein, 7 aa, Artificial Sequence
  source: /note=“Description of Artificial Sequence Synthetic peptide”
RSADLSR

SEQ ID NO: 44 - protein, 7 aa, Artificial Sequence
  source: /note=“Description of Artificial Sequence Synthetic peptide”
QRSNLNI

SEQ ID NO: 45 - DNA, 20 bp, Homo sapiens
ggacggcgacgaccagaagg

SEQ ID NO: 46 - protein, 7 aa, Artificial Sequence
  source: /note=“Description of Artificial Sequence Synthetic peptide”
RSDNLSV

SEQ ID NO: 47 - protein, 7 aa, Artificial Sequence
  source: /note=“Description of Artificial Sequence Synthetic peptide”
ASWTLTQ

SEQ ID NO: 48 - protein, 7 aa, Artificial Sequence
  source: /note=“Description of Artificial Sequence Synthetic peptide”
DRSNLTR

SEQ ID NO: 49 - protein, 7 aa, Artificial Sequence
  source: /note=“Description of Artificial Sequence Synthetic peptide”
DRSHLTR

SEQ ID NO: 50 - DNA, 20 bp, Homo sapiens
cgcgacgcggaagtgaggtg

SEQ ID NO: 51 - protein, 7 aa, Artificial Sequence
  source: /note=“Description of Artificial Sequence Synthetic peptide”
LRHHLTR

SEQ ID NO: 52 - protein, 7 aa, Artificial Sequence
  source: /note=“Description of Artificial Sequence Synthetic peptide”
VAEYRYK

SEQ ID NO: 53 - protein, 7 aa, Artificial Sequence
  source: /note=“Description of Artificial Sequence Synthetic peptide”
RHSHLTS

SEQ ID NO: 54 - protein, 7 aa, Artificial Sequence
  source: /note=“Description of Artificial Sequence Synthetic peptide”
RSDTLSA

SEQ ID NO: 55 - DNA, 20 bp, Homo sapiens
cgctcacgagggtggaaagg

SEQ ID NO: 56 - protein, 7 aa, Artificial Sequence
  source: /note=“Description of Artificial Sequence Synthetic peptide”
QSGHLSR

SEQ ID NO: 57 - protein, 7 aa, Artificial Sequence
  source: /note=“Description of Artificial Sequence Synthetic peptide”
DAHHLHR

SEQ ID NO: 58 - protein, 7 aa, Artificial Sequence
  source: /note=“Description of Artificial Sequence Synthetic peptide”
RSANLTR

SEQ ID NO: 59 - protein, 7 aa, Artificial Sequence
  source: /note=“Description of Artificial Sequence Synthetic peptide”
DRSNRTT

SEQ ID NO: 60 - protein, 7 aa, Artificial Sequence
  source: /note=“Description of Artificial Sequence Synthetic peptide”
HRSSLRR

SEQ ID NO: 61 - DNA, 20 bp, Homo sapiens
attcgacgacaggttagcgg

SEQ ID NO: 62 - protein, 7 aa, Artificial Sequence
  source: /note=“Description of Artificial Sequence Synthetic peptide”
RSDDLTR

SEQ ID NO: 63 - protein, 7 aa, Artificial Sequence
  source: /note=“Description of Artificial Sequence Synthetic peptide”
QKFPRDS

SEQ ID NO: 64 - protein, 7 aa, Artificial Sequence
  source: /note=“Description of Artificial Sequence Synthetic peptide”
RSDHLTQ

SEQ ID NO: 65 - protein, 7 aa, Artificial Sequence
  source: /note=“Description of Artificial Sequence Synthetic peptide”
DSANLSR

SEQ ID NO: 66 - protein, 7 aa, Artificial Sequence
  source: /note=“Description of Artificial Sequence Synthetic peptide”
DRSNLSR

SEQ ID NO: 67 - protein, 7 aa, Artificial Sequence
  source: /note=“Description of Artificial Sequence Synthetic peptide”
DRTSLKW

SEQ ID NO: 68 - DNA, 21 bp, Homo sapiens
ggccgctgtgagccggcgacg

SEQ ID NO: 69 - protein, 7 aa, Artificial Sequence
  source: /note=“Description of Artificial Sequence Synthetic peptide”
HLCCLGR

SEQ ID NO: 70 - protein, 7 aa, Artificial Sequence
  source: /note=“Description of Artificial Sequence Synthetic peptide”
RSCCLHL

SEQ ID NO: 71 - protein, 7 aa, Artificial Sequence
  source: /note=“Description of Artificial Sequence Synthetic peptide”
RNASRTR

SEQ ID NO: 72 - protein, 7 aa, Artificial Sequence
  source: /note=“Description of Artificial Sequence Synthetic peptide”
QSSDLSR

SEQ ID NO: 73 - protein, 7 aa, Artificial Sequence
  source: /note=“Description of Artificial Sequence Synthetic peptide”
HRKSLSR

SEQ ID NO: 74 - DNA, 21 bp, Homo sapiens
aggcggcacccgggggggact

SEQ ID NO: 75 - protein, 7 aa, Artificial Sequence
  source: /note=“Description of Artificial Sequence Synthetic peptide”
DRSHLAR

SEQ ID NO: 76 - protein, 7 aa, Artificial Sequence
  source: /note=“Description of Artificial Sequence Synthetic peptide”
RSDHLSE

SEQ ID NO: 77 - protein, 7 aa, Artificial Sequence
  source: /note=“Description of Artificial Sequence Synthetic peptide”
TSSDRTK

SEQ ID NO: 78 - protein, 7 aa, Artificial Sequence
  source: /note=“Description of Artificial Sequence Synthetic peptide”
DRSHLSR

SEQ ID NO: 79 - DNA, 21 bp, Homo sapiens
gagcgcacaggaagggcggag

SEQ ID NO: 80 - protein, 7 aa, Artificial Sequence
  source: /note=“Description of Artificial Sequence Synthetic peptide”
AKWNLDA

SEQ ID NO: 81 - protein, 7 aa, Artificial Sequence
  source: /note=“Description of Artificial Sequence Synthetic peptide”
TQGYLRK

SEQ ID NO: 82 - DNA, 21 bp, Homo sapiens
ggcggggaggagtcggagttg

SEQ ID NO: 83 - protein, 7 aa, Artificial Sequence
  source: /note=“Description of Artificial Sequence Synthetic peptide”
TSGSLSR

SEQ ID NO: 84 - protein, 7 aa, Artificial Sequence
  source: /note=“Description of Artificial Sequence Synthetic peptide”
QAGHLAK

SEQ ID NO: 85 - protein, 7 aa, Artificial Sequence
  source: /note=“Description of Artificial Sequence Synthetic peptide”
DRSARTR

SEQ ID NO: 86 - protein, 7 aa, Artificial Sequence
  source: /note=“Description of Artificial Sequence Synthetic peptide”
QSSDLRR

SEQ ID NO: 87 - DNA, 21 bp, Homo sapiens
ggtcgtgggcaccgggagggg

SEQ ID NO: 88 - protein, 7 aa, Artificial Sequence
  source: /note=“Description of Artificial Sequence Synthetic peptide”
QHAHRNT

SEQ ID NO: 89 - protein, 7 aa, Artificial Sequence
  source: /note=“Description of Artificial Sequence Synthetic peptide”
RPDTLRD

SEQ ID NO: 90 - protein, 7 aa, Artificial Sequence
  source: /note=“Description of Artificial Sequence Synthetic peptide”
QNATRTK

SEQ ID NO: 91 - protein, 7 aa, Artificial Sequence
  source: /note=“Description of Artificial Sequence Synthetic peptide”
RSDALSR

SEQ ID NO: 92 - protein, 7 aa, Artificial Sequence
  source: /note=“Description of Artificial Sequence Synthetic peptide”
DRSALAR

SEQ ID NO: 93 - DNA, 21 bp, Homo sapiens
ggctgcttctccgggatccgc

SEQ ID NO: 94 - protein, 7 aa, Artificial Sequence
  source: /note=“Description of Artificial Sequence Synthetic peptide”
NNRDLIN

SEQ ID NO: 95 - protein, 7 aa, Artificial Sequence
  source: /note=“Description of Artificial Sequence Synthetic peptide”
TSSNLSR

SEQ ID NO: 96 - protein, 7 aa, Artificial Sequence
  source: /note=“Description of Artificial Sequence Synthetic peptide”
RSDTLSE

SEQ ID NO: 97 - protein, 7 aa, Artificial Sequence
  source: /note=“Description of Artificial Sequence Synthetic peptide”
NNRDRTK

SEQ ID NO: 98 - protein, 7 aa, Artificial Sequence
  source: /note=“Description of Artificial Sequence Synthetic peptide”
HRSTRNR

SEQ ID NO: 99 - DNA, 21 bp, Homo sapiens
ggaggacggcgacgaccagaa

SEQ ID NO: 100 - protein, 7 aa, Artificial Sequence
  source: /note=“Description of Artificial Sequence Synthetic peptide”
QNAHRKT

SEQ ID NO: 101 - protein, 7 aa, Artificial Sequence
  source: /note=“Description of Artificial Sequence Synthetic peptide”
DNSTRIK

SEQ ID NO: 102 - protein, 7 aa, Artificial Sequence
  source: /note=“Description of Artificial Sequence Synthetic peptide”
TSANLSR

SEQ ID NO: 103 - DNA, 20 bp, Homo sapiens
agagggggcgagcgaccgag

SEQ ID NO: 104 - protein, 7 aa, Artificial Sequence
  source: /note=“Description of Artificial Sequence Synthetic peptide”
QRNHRTT

SEQ ID NO: 105 - DNA, 21 bp, Homo sapiens
cgtcgcggcgctcggtcgctc

SEQ ID NO: 106 - protein, 7 aa, Artificial Sequence
  source: /note=“Description of Artificial Sequence Synthetic peptide”
DRSYRNT

SEQ ID NO: 107 - protein, 7 aa, Artificial Sequence
  source: /note=“Description of Artificial Sequence Synthetic peptide”
RRSDLKR

SEQ ID NO: 108 - DNA, 21 bp, Homo sapiens
ctgcgctgcagcccgcacgca

SEQ ID NO: 109 - protein, 7 aa, Artificial Sequence
  source: /note=“Description of Artificial Sequence Synthetic peptide”
DSSTRKT

SEQ ID NO: 110 - protein, 7 aa, Artificial Sequence
  source: /note=“Description of Artificial Sequence Synthetic peptide”
QSATRTK

SEQ ID NO: 111 - protein, 7 aa, Artificial Sequence
  source: /note=“Description of Artificial Sequence Synthetic peptide”
AASNRSK

SEQ ID NO: 112 - DNA, 21 bp, Homo sapiens
cgtcgaatggccactcccagt

SEQ ID NO: 113 - protein, 7 aa, Artificial Sequence
  source: /note=“Description of Artificial Sequence Synthetic peptide”
RSDNLSQ

SEQ ID NO: 114 - protein, 7 aa, Artificial Sequence
  source: /note=“Description of Artificial Sequence Synthetic peptide”
ASNDRKK

SEQ ID NO: 115 - protein, 7 aa, Artificial Sequence
  source: /note=“Description of Artificial Sequence Synthetic peptide”
YDYGRYT

SEQ ID NO: 116 - DNA, 21 bp, Homo sapiens
gtacctgtggatctaaacggg

SEQ ID NO: 117 - protein, 7 aa, Artificial Sequence
  source: /note=“Description of Artificial Sequence Synthetic peptide”
QSNHRKT

SEQ ID NO: 118 - protein, 7 aa, Artificial Sequence
  source: /note=“Description of Artificial Sequence Synthetic peptide”
VYEGLKK

SEQ ID NO: 119 - protein, 7 aa, Artificial Sequence
  source: /note=“Description of Artificial Sequence Synthetic peptide”
RSDVLSE

SEQ ID NO: 120 - protein, 7 aa, Artificial Sequence
  source: /note=“Description of Artificial Sequence Synthetic peptide”
DRSNRIK

SEQ ID NO: 121 - DNA, 21 bp, Homo sapiens
aaagtgggggcgcttttgcgc

SEQ ID NO: 122 - protein, 7 aa, Artificial Sequence
  source: /note=“Description of Artificial Sequence Synthetic peptide”
RRADLSR

SEQ ID NO: 123 - protein, 7 aa, Artificial Sequence
  source: /note=“Description of Artificial Sequence Synthetic peptide”
YHWYLKK

SEQ ID NO: 124 - protein, 7 aa, Artificial Sequence
  source: /note=“Description of Artificial Sequence Synthetic peptide”
RSDHLSQ

SEQ ID NO: 125 - protein, 7 aa, Artificial Sequence
  source: /note=“Description of Artificial Sequence Synthetic peptide”
TSANRTT

SEQ ID NO: 126 - DNA, 20 bp, Homo sapiens
gcaaggcgggaaagtggggg

SEQ ID NO: 127 - protein, 7 aa, Artificial Sequence
  source: /note=“Description of Artificial Sequence Synthetic peptide”
RSDHLST

SEQ ID NO: 128 - protein, 7 aa, Artificial Sequence
  source: /note=“Description of Artificial Sequence Synthetic peptide”
RSDALAR

SEQ ID NO: 129 - protein, 7 aa, Artificial Sequence
  source: /note=“Description of Artificial Sequence Synthetic peptide”
QSANRTT

SEQ ID NO: 130 - protein, 7 aa, Artificial Sequence
  source: /note=“Description of Artificial Sequence Synthetic peptide”
QSGNLHV

SEQ ID NO: 131 - DNA, 21 bp, Homo sapiens
gcgcaaggcgggaaagtgggg

SEQ ID NO: 132 - protein, 7 aa, Artificial Sequence
  source: /note=“Description of Artificial Sequence Synthetic peptide”
AKHHRKT

SEQ ID NO: 133 - protein, 7 aa, Artificial Sequence
  source: /note=“Description of Artificial Sequence Synthetic peptide”
RSDNLSA

SEQ ID NO: 134 - protein, 7 aa, Artificial Sequence
  source: /note=“Description of Artificial Sequence Synthetic peptide”
QNRDRKN

SEQ ID NO: 135 - DNA, 20 bp, Homo sapiens
cgcgcaaggcgggaaagtgg

SEQ ID NO: 136 - protein, 7 aa, Artificial Sequence
  source: /note=“Description of Artificial Sequence Synthetic peptide”
RSDSLSV

SEQ ID NO: 137 - protein, 7 aa, Artificial Sequence
  source: /note=“Description of Artificial Sequence Synthetic peptide”
QNANRKT

SEQ ID NO: 138 - DNA, 20 bp, Homo sapiens
ggagcacgctgcagggaaag

SEQ ID NO: 139 - protein, 7 aa, Artificial Sequence
  source: /note=“Description of Artificial Sequence Synthetic peptide”
DQSNLRA

SEQ ID NO: 140 - DNA, 21 bp, Homo sapiens
agcacgctgcagggaaagcag

SEQ ID NO: 141 - DNA, 20 bp, Homo sapiens
tgcagggaaagcagcgagcg

SEQ ID NO: 142 - DNA, 20 bp, Homo sapiens
gtgggggcggagagccgagg

SEQ ID NO: 143 - protein, 7 aa, Artificial Sequence
  source: /note=“Description of Artificial Sequence Synthetic peptide”
RSDNLAR

SEQ ID NO: 144 - protein, 7 aa, Artificial Sequence
  source: /note=“Description of Artificial Sequence Synthetic peptide”
DRSVLHR

SEQ ID NO: 145 - protein, 7 aa, Artificial Sequence
  source: /note=“Description of Artificial Sequence Synthetic peptide”
QSAHRKN

SEQ ID NO: 146 - protein, 7 aa, Artificial Sequence
  source: /note=“Description of Artificial Sequence Synthetic peptide”
RNDHRTT

SEQ ID NO: 147 - DNA, 21 bp, Homo sapiens
gagagggggcgagcgaccgag

SEQ ID NO: 148 - protein, 7 aa, Artificial Sequence
  source: /note=“Description of Artificial Sequence Synthetic peptide”
QGAHLGA

SEQ ID NO: 149 - DNA, 21 bp, Homo sapiens
gcagcgcagaccccggcccgg

SEQ ID NO: 150 - protein, 7 aa, Artificial Sequence
  source: /note=“Description of Artificial Sequence Synthetic peptide”
RSDALSV

SEQ ID NO: 151 - protein, 7 aa, Artificial Sequence
  source: /note=“Description of Artificial Sequence Synthetic peptide”
DSSHRTR

SEQ ID NO: 152 - protein, 7 aa, Artificial Sequence
  source: /note=“Description of Artificial Sequence Synthetic peptide”
DNRDLST

SEQ ID NO: 153 - DNA, 21 bp, Homo sapiens
tctcggaggggccgggccggg

SEQ ID NO: 154 - protein, 7 aa, Artificial Sequence
  source: /note=“Description of Artificial Sequence Synthetic peptide”
DNSHRTR

SEQ ID NO: 155 - protein, 7 aa, Artificial Sequence
  source: /note=“Description of Artificial Sequence Synthetic peptide”
RSTHLVR

SEQ ID NO: 156 - DNA, 20 bp, Homo sapiens
ggacgctctcggaggggccg

SEQ ID NO: 157 - protein, 7 aa, Artificial Sequence
  source: /note=“Description of Artificial Sequence Synthetic peptide”
QESDLVR

SEQ ID NO: 158 - DNA, 20 bp, Homo sapiens
aaaaggagcgcacaggaagg

SEQ ID NO: 159 - protein, 7 aa, Artificial Sequence
  source: /note=“Description of Artificial Sequence Synthetic peptide”
RRAHLNQ

SEQ ID NO: 160 - protein, 7 aa, Artificial Sequence
  source: /note=“Description of Artificial Sequence Synthetic peptide”
QRVALQA

SEQ ID NO: 161 - DNA, 21 bp, Homo sapiens
ggaaaaggagcgcacaggaag

SEQ ID NO: 162 - DNA, 21 bp, Homo sapiens
tcaggtaagtacctgtggatc

SEQ ID NO: 163 - protein, 7 aa, Artificial Sequence
  source: /note=“Description of Artificial Sequence Synthetic peptide”
RSELLQS

SEQ ID NO: 164 - protein, 7 aa, Artificial Sequence
  source: /note=“Description of Artificial Sequence Synthetic peptide”
ARSTRIT

SEQ ID NO: 165 - protein, 7 aa, Artificial Sequence
  source: /note=“Description of Artificial Sequence Synthetic peptide”
QSGSLTR

SEQ ID NO: 166 - DNA, 20 bp, Homo sapiens
tctgggaggtgagtacttgt

SEQ ID NO: 167 - protein, 7 aa, Artificial Sequence
  source: /note=“Description of Artificial Sequence Synthetic peptide”
RPYTLRL

SEQ ID NO: 168 - protein, 7 aa, Artificial Sequence
  source: /note=“Description of Artificial Sequence Synthetic peptide”
DNSNRIK

SEQ ID NO: 169 - protein, 7 aa, Artificial Sequence
  source: /note=“Description of Artificial Sequence Synthetic peptide”
TSGHLSR

SEQ ID NO: 170 - protein, 7 aa, Artificial Sequence
  source: /note=“Description of Artificial Sequence Synthetic peptide”
RKWTLQG

SEQ ID NO: 171 - DNA, 20 bp, Homo sapiens
ccaaagggacaagtactcac

SEQ ID NO: 172 - protein, 7 aa, Artificial Sequence
  source: /note=“Description of Artificial Sequence Synthetic peptide”
QSADRTK

SEQ ID NO: 173 - protein, 7 aa, Artificial Sequence
  source: /note=“Description of Artificial Sequence Synthetic peptide”
DSANRIK

SEQ ID NO: 174 - protein, 7 aa, Artificial Sequence
  source: /note=“Description of Artificial Sequence Synthetic peptide”
QNANRIT

SEQ ID NO: 175 - DNA, 21 bp, Homo sapiens
cccaaagggacaagtactcac

SEQ ID NO: 176 - protein, 7 aa, Artificial Sequence
  source: /note=“Description of Artificial Sequence Synthetic peptide”
RSANLSV

SEQ ID NO: 177 - protein, 7 aa, Artificial Sequence
  source: /note=“Description of Artificial Sequence Synthetic peptide”
DRANLSR

SEQ ID NO: 178 - protein, 7 aa, Artificial Sequence
  source: /note=“Description of Artificial Sequence Synthetic peptide”
QKATRIT

SEQ ID NO: 179 - protein, 298 aa, Artificial Sequence
  source: /note=“Description of Artificial Sequence Synthetic polypeptide”
MAPKKKRKVGVPAAMAERPFQCRICMRNFSQSGHLARHIRTHTGEKPFACDICGRKFAQRKDLTQHTKIHTHPRAPIPKPFQCRICMRNFSRSDHLSRHIRTHTGEKPFACDICGRKFATRDGLRQHTKIHTGSQKPFQCRICMRNFSRSANLARHIRTHTGEKPFACDICGRKFADSSDRKKHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 180 - protein, 294 aa, Artificial Sequence
  source: /note=“Description of Artificial Sequence Synthetic polypeptide”
MAPKKKRKVGVPAAMAERPFQCRICMRNFSRSDNLSTHIRTHTGEKPFACDICGRKFARSDNLTRHTKIHTGSQKPFQCRICMRNFSDRSDLSRHIRTHTGEKPFACDICGRKFARREHLRAHTKIHTGSQKPFQCRICMRNFSTSGNLTRHIRTHTGEKPFACDICGRKFAQRSHLSDHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 181 - protein, 298 aa, Artificial Sequence
  source: /note=“Description of Artificial Sequence Synthetic polypeptide”
MAPKKKRKVGVPAAMAERPFQCRICMRNFSERGTLARHIRTHTGEKPFACDICGRKFAMACCRYAHTKIHTGSQKPFQCRICMRNFSRSADLTRHIRTHTGEKPFACDICGRKFAQSGDLTRHTKIHTHPRAPIPKPFQCRICMRNFSQSGNLARHIRTHTGEKPFACDICGRKFARQEHRVAHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 182 - protein, 294 aa, Artificial Sequence
  source: /note=“Description of Artificial Sequence Synthetic polypeptide”
MAPKKKRKVGVPAAMAERPFQCRICMRNFSRSAHLSRHIRTHTGEKPFACDICGRKFADRSDLSRHTKIHTGSQKPFQCRICMRNFSMSCCRSAHIRTHTGEKPFACDICGRKFARSADLSRHTKIHTGSQKPFQCRICMRNFSQSGDLTRHIRTHTGEKPFACDICGRKFAQRSNLNIHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 183 - protein, 294 aa, Artificial Sequence
  source: /note=“Description of Artificial Sequence Synthetic polypeptide”
MAPKKKRKVGVPAAMAERPFQCRICMRNFSRSDNLSVHIRTHTGEKPFACDICGRKFAASWTLTQHTKIHTGSQKPFQCRICMRNFSDRSDLSRHIRTHTGEKPFACDICGRKFADRSNLTRHTKIHTGSQKPFQCRICMRNFSDRSHLTRHIRTHTGEKPFACDICGRKFADRSNLTRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 184 - protein, 294 aa, Artificial Sequence
  source: /note=“Description of Artificial Sequence Synthetic polypeptide”
MAPKKKRKVGVPAAMAERPFQCRICMRNFSLRHHLTRHIRTHTGEKPFACDICGRKFAVAEYRYKHTKIHTGSQKPFQCRICMRNFSRSDNLSVHIRTHTGEKPFACDICGRKFARHSHLTSHTKIHTGSQKPFQCRICMRNFSRSDTLSAHIRTHTGEKPFACDICGRKFARSADLSRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 185 - protein, 294 aa, Artificial Sequence
  source: /note=“Description of Artificial Sequence Synthetic polypeptide”
MAPKKKRKVGVPAAMAERPFQCRICMRNFSRSDNLSTHIRTHTGEKPFACDICGRKFAQSGHLSRHTKIHTGSQKPFQCRICMRNFSDAHHLHRHIRTHTGEKPFACDICGRKFARSANLTRHTKIHTGSQKPFQCRICMRNFSDRSNRTTHIRTHTGEKPFACDICGRKFAHRSSLRRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 186 - protein, 294 aa, Artificial Sequence
  source: /note=“Description of Artificial Sequence Synthetic polypeptide”
MAPKKKRKVGVPAAMAERPFQCRICMRNFSRSDDLTRHIRTHTGEKPFACDICGRKFAQKFPRDSHTKIHTGSQKPFQCRICMRNFSRSDHLTQHIRTHTGEKPFACDICGRKFADSANLSRHTKIHTGSQKPFQCRICMRNFSDRSNLSRHIRTHTGEKPFACDICGRKFADRTSLKWHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 187 - protein, 298 aa, Artificial Sequence
  source: /note=“Description of Artificial Sequence Synthetic polypeptide”
MAPKKKRKVGVPAAMAERPFQCRICMRNFSDRSNLSRHIRTHTGEKPFACDICGRKFAHLCCLGRHTKIHTHPRAPIPKPFQCRICMRNFSRSCCLHLHIRTHTGEKPFACDICGRKFARNASRTRHTKIHTGSQKPFQCRICMRNFSQSSDLSRHIRTHTGEKPFACDICGRKFAHRKSLSRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 188 - protein, 298 aa, Artificial Sequence
  source: /note=“Description of Artificial Sequence Synthetic polypeptide”
MAPKKKRKVGVPAAMAERPFQCRICMRNFSDRSNLSRHIRTHTGEKPFACDICGRKFADRSHLARHTKIHTHPRAPIPKPFQCRICMRNFSRSDHLSEHIRTHTGEKPFACDICGRKFATSSDRTKHTKIHTGSQKPFQCRICMRNFSDRSHLSRHIRTHTGEKPFACDICGRKFADRSHLARHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 189 - protein, 298 aa, Artificial Sequence
  source: /note=“Description of Artificial Sequence Synthetic polypeptide”
MAPKKKRKVGVPAAMAERPFQCRICMRNFSDRSHLTRHIRTHTGEKPFACDICGRKFADRSHLARHTKIHTHPRAPIPKPFQCRICMRNFSQSGNLARHIRTHTGEKPFACDICGRKFAAKWNLDAHTKIHTGSQKPFQCRICMRNFSQSGDLTRHIRTHTGEKPFACDICGRKFATQGYLRKHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 190 - protein, 298 aa, Artificial Sequence
  source: /note=“Description of Artificial Sequence Synthetic polypeptide”
MAPKKKRKVGVPAAMAERPFQCRICMRNFSTSGSLSRHIRTHTGEKPFACDICGRKFAQAGHLAKHTKIHTGSQKPFQCRICMRNFSDRSARTRHIRTHTGEKPFACDICGRKFAQSGHLSRHTKIHTHPRAPIPKPFQCRICMRNFSRSAHLSRHIRTHTGEKPFACDICGRKFAQSSDLRRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 191 - protein, 298 aa, Artificial Sequence
  source: /note=“Description of Artificial Sequence Synthetic polypeptide”
MAPKKKRKVGVPAAMAERPFQCRICMRNFSRSDHLSRHIRTHTGEKPFACDICGRKFAQHAHRNTHTKIHTGSQKPFQCRICMRNFSRPDTLRDHIRTHTGEKPFACDICGRKFAQNATRTKHTKIHTHPRAPIPKPFQCRICMRNFSRSDALSRHIRTHTGEKPFACDICGRKFADRSALARHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 192 - protein, 298 aa, Artificial Sequence
  source: /note=“Description of Artificial Sequence Synthetic polypeptide”
MAPKKKRKVGVPAAMAERPFQCRICMRNFSNNRDLINHIRTHTGEKPFACDICGRKFATSSNLSRHTKIHTHPRAPIPKPFQCRICMRNFSRSDTLSEHIRTHTGEKPFACDICGRKFANNRDRTKHTKIHTGSQKPFQCRICMRNFSQSSDLSRHIRTHTGEKPFACDICGRKFAHRSTRNRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 193 - protein, 298 aa, Artificial Sequence
  source: /note=“Description of Artificial Sequence Synthetic polypeptide”
MAPKKKRKVGVPAAMAERPFQCRICMRNFSQNAHRKTHIRTHTGEKPFACDICGRKFADNSTRIKHTKIHTHPRAPIPKPFQCRICMRNFSDRSNLSRHIRTHTGEKPFACDICGRKFAHLCCLGRHTKIHTGSQKPFQCRICMRNFSDRSNLSRHIRTHTGEKPFACDICGRKFATSANLSRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 194 - protein, 294 aa, Artificial Sequence
  source: /note=“Description of Artificial Sequence Synthetic polypeptide”
MAPKKKRKVGVPAAMAERPFQCRICMRNFSQRNHRTTHIRTHTGEKPFACDICGRKFADRSNLTRHTKIHTGSQKPFQCRICMRNFSMSCCRSAHIRTHTGEKPFACDICGRKFARSADLSRHTKIHTGSQKPFQCRICMRNFSRSAHLSRHIRTHTGEKPFACDICGRKFATSANLSRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 195 - protein, 298 aa, Artificial Sequence
  source: /note=“Description of Artificial Sequence Synthetic polypeptide”
MAPKKKRKVGVPAAMAERPFQCRICMRNFSQSSDLSRHIRTHTGEKPFACDICGRKFADRSALARHTKIHTHPRAPIPKPFQCRICMRNFSDRSYRNTHIRTHTGEKPFACDICGRKFARRSDLKRHTKIHTGSQKPFQCRICMRNFSRSADLTRHIRTHTGEKPFACDICGRKFADRSALARHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 196 - protein, 298 aa, Artificial Sequence
  source: /note=“Description of Artificial Sequence Synthetic polypeptide”
MAPKKKRKVGVPAAMAERPFQCRICMRNFSDSSTRKTHIRTHTGEKPFACDICGRKFAQSATRTKHTKIHTHPRAPIPKPFQCRICMRNFSERGTLARHIRTHTGEKPFACDICGRKFAQSGDLTRHTKIHTGSQKPFQCRICMRNFSQSSDLSRHIRTHTGEKPFACDICGRKFAAASNRSKHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 197 - protein, 298 aa, Artificial Sequence
  source: /note=“Description of Artificial Sequence Synthetic polypeptide”
MAPKKKRKVGVPAAMAERPFQCRICMRNFSRSDNLSQHIRTHTGEKPFACDICGRKFAASNDRKKHTKIHTGSQKPFQCRICMRNFSYDYGRYTHIRTHTGEKPFACDICGRKFADRSHLARHTKIHTHPRAPIPKPFQCRICMRNFSQSGNLARHIRTHTGEKPFACDICGRKFADRSALARHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 198 - protein, 298 aa, Artificial Sequence
  source: /note=“Description of Artificial Sequence Synthetic polypeptide”
MAPKKKRKVGVPAAMAERPFQCRICMRNFSRSDHLSEHIRTHTGEKPFACDICGRKFAQSNHRKTHTKIHTGSQKPFQCRICMRNFSVYEGLKKHIRTHTGEKPFACDICGRKFAQSGHLSRHTKIHTHPRAPIPKPFQCRICMRNFSRSDVLSEHIRTHTGEKPFACDICGRKFADRSNRIKHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 199 - protein, 298 aa, Artificial Sequence
  source: /note=“Description of Artificial Sequence Synthetic polypeptide”
MAPKKKRKVGVPAAMAERPFQCRICMQNFSRRADLSRHIRTHTGEKPFACDICGRKFAYHWYLKKHTKIHTGSQKPFQCRICMQNFSQSSDLSRHIRTHTGEKPFACDICGRKFADRSHLARHTKIHTHPRAPIPKPFQCRICMQNFSRSDHLSQHIRTHTGEKPFACDICGRKFATSANRTTHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 200 - protein, 294 aa, Artificial Sequence
  source: /note=“Description of Artificial Sequence Synthetic polypeptide”
MAPKKKRKVGVPAAMAERPFQCRICMQNFSRSDHLSTHIRTHTGEKPFACDICGRKFARSDALARHTKIHTGSQKPFQCRICMQNFSQSANRTTHIRTHTGEKPFACDICGRKFARSAHLSRHTKIHTGSQKPFQCRICMRNFSDRSHLTRHIRTHTGEKPFACDICGRKFAQSGNLHVHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 201 - protein, 294 aa, Artificial Sequence
  source: /note=“Description of Artificial Sequence Synthetic polypeptide”
MAPKKKRKVGVPAAMAERPFQCRICMQNFSRSDHLSTHIRTHTGEKPFACDICGRKFARSDALARHTKIHTGSQKPFQCRICMQNFSQSANRTTHIRTHTGEKPFACDICGRKFARSAHLSRHTKIHTGSQKPFQCRICMQNFSDRSHLTRHIRTHTGEKPFACDICGRKFAQSGNLHVHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 202 - protein, 298 aa, Artificial Sequence
  source: /note=“Description of Artificial Sequence Synthetic polypeptide”
MAPKKKRKVGVPAAMAERPFQCRICMQNFSRSDHLSEHIRTHTGEKPFACDICGRKFAAKHHRKTHTKIHTGSQKPFQCRICMRNFSQSGNLARHIRTHTGEKPFACDICGRKFARQEHRVAHTKIHTHPRAPIPKPFQCRICMRNFSRSDNLSAHIRTHTGEKPFACDICGRKFAQNRDRKNHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 203 - protein, 294 aa, Artificial Sequence
  source: /note=“Description of Artificial Sequence Synthetic polypeptide”
MAPKKKRKVGVPAAMAERPFQCRICMQNFSRSDSLSVHIRTHTGEKPFACDICGRKFAQNANRKTHTKIHTGSQKPFQCRICMRNFSRSAHLSRHIRTHTGEKPFACDICGRKFADRSHLARHTKIHTGSQKPFQCRICMRNFSQSANRTTHIRTHTGEKPFACDICGRKFARRSDLKRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 204 - protein, 294 aa, Artificial Sequence
  source: /note=“Description of Artificial Sequence Synthetic polypeptide”
MAPKKKRKVGVPAAMAERPFQCRICMQNFSRSDSLSVHIRTHTGEKPFACDICGRKFAQNANRKTHTKIHTGSQKPFQCRICMQNFSRSAHLSRHIRTHTGEKPFACDICGRKFADRSHLARHTKIHTGSQKPFQCRICMRNFSQSANRTTHIRTHTGEKPFACDICGRKFARRSDLKRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 205 - protein, 294 aa, Artificial Sequence
  source: /note=“Description of Artificial Sequence Synthetic polypeptide”
MAPKKKRKVGVPAAMAERPFQCRICMQNFSQSANRTTHIRTHTGEKPFACDICGRKFARSAHLSRHTKIHTGSQKPFQCRICMQNFSQSGDLTRHIRTHTGEKPFACDICGRKFAQSSDLRRHTKIHTGSQKPFQCRICMRNFSDQSNLRAHIRTHTGEKPFACDICGRKFARSANLTRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 206 - protein, 294 aa, Artificial Sequence
  source: /note=“Description of Artificial Sequence Synthetic polypeptide”
MAPKKKRKVGVPAAMAERPFQCRICMQNFSQSANRTTHIRTHTGEKPFACDICGRKFARSAHLSRHTKIHTGSQKPFQCRICMQNFSQSGDLTRHIRTHTGEKPFACDICGRKFAQSSDLRRHTKIHTGSQKPFQCRICMQNFSDQSNLRAHIRTHTGEKPFACDICGRKFARSANLTRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 207 - protein, 298 aa, Artificial Sequence
  source: /note=“Description of Artificial Sequence Synthetic polypeptide”
MAPKKKRKVGVPAAMAERPFQCRICMQNFSQSGDLTRHIRTHTGEKPFACDICGRKFAQRSNLNIHTKIHTGSQKPFQCRICMRNFSRSAHLSRHIRTHTGEKPFACDICGRKFAQSGDLTRHTKIHTHPRAPIPKPFQCRICMRNFSDSSTRKTHIRTHTGEKPFACDICGRKFAQSATRTKHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 208 - protein, 298 aa, Artificial Sequence
  source: /note=“Description of Artificial Sequence Synthetic polypeptide”
MAPKKKRKVGVPAAMAERPFQCRICMQNFSQSGDLTRHIRTHTGEKPFACDICGRKFAQRSNLNIHTKIHTGSQKPFQCRICMQNFSRSAHLSRHIRTHTGEKPFACDICGRKFAQSGDLTRHTKIHTHPRAPIPKPFQCRICMRNFSDSSTRKTHIRTHTGEKPFACDICGRKFAQSATRTKHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 209 - protein, 294 aa, Artificial Sequence
  source: /note=“Description of Artificial Sequence Synthetic polypeptide”
MAPKKKRKVGVPAAMAERPFQCRICMQNFSMSCCRSAHIRTHTGEKPFACDICGRKFARSADLSRHTKIHTGSQKPFQCRICMQNFSQSGDLTRHIRTHTGEKPFACDICGRKFAQRSNLNIHTKIHTGSQKPFQCRICMQNFSRSAHLSRHIRTHTGEKPFACDICGRKFAQSGDLTRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 210 - protein, 294 aa, Artificial Sequence
  source: /note=“Description of Artificial Sequence Synthetic polypeptide”
MAPKKKRKVGVPAAMAERPFQCRICMQNFSRSDNLARHIRTHTGEKPFACDICGRKFADRSVLHRHTKIHTGSQKPFQCRICMRNFSQSAHRKNHIRTHTGEKPFACDICGRKFARHSHLTSHTKIHTGSQKPFQCRICMRNFSRSDHLSEHIRTHTGEKPFACDICGRKFARNDHRTTHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 211 - protein, 298 aa, Artificial Sequence
  source: /note=“Description of Artificial Sequence Synthetic polypeptide”
MAPKKKRKVGVPAAMAERPFQCRICMQNFSQRNHRTTHIRTHTGEKPFACDICGRKFADRSNLTRHTKIHTGSQKPFQCRICMRNFSMSCCRSAHIRTHTGEKPFACDICGRKFARSADLSRHTKIHTHPRAPIPKPFQCRICMRNFSRSDHLSRHIRTHTGEKPFACDICGRKFAQGAHLGAHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 212 - protein, 294 aa, Artificial Sequence
  source: /note=“Description of Artificial Sequence Synthetic polypeptide”
MAPKKKRKVGVPAAMAERPFQCRICMQNFSRSDALSVHIRTHTGEKPFACDICGRKFADSSHRTRHTKIHTGSQKPFQCRICMRNFSDNRDLSTHIRTHTGEKPFACDICGRKFADRSNLTRHTKIHTGSQKPFQCRICMRNFSQSGDLTRHIRTHTGEKPFACDICGRKFATQGYLRKHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 213 - protein, 298 aa, Artificial Sequence
  source: /note=“Description of Artificial Sequence Synthetic polypeptide”
MAPKKKRKVGVPAAMAERPFQCRICMQNFSRSDHLSQHIRTHTGEKPFACDICGRKFADNSHRTRHTKIHTHPRAPIPKPFQCRICMRNFSDRSDLSRHIRTHTGEKPFACDICGRKFARSTHLVRHTKIHTGSQKPFQCRICMRNFSDRSHLTRHIRTHTGEKPFACDICGRKFADRSYRNTHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 214 - protein, 294 aa, Artificial Sequence
  source: /note=“Description of Artificial Sequence Synthetic polypeptide”
MAPKKKRKVGVPAAMAERPFQCRICMQNFSDRSDLSRHIRTHTGEKPFACDICGRKFARSTHLVRHTKIHTGSQKPFQCRICMRNFSDRSHLTRHIRTHTGEKPFACDICGRKFADRSYRNTHTKIHTGSQKPFQCRICMRNFSQESDLVRHIRTHTGEKPFACDICGRKFADRSNLTRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 215 - protein, 294 aa, Artificial Sequence
  source: /note=“Description of Artificial Sequence Synthetic polypeptide”
MAPKKKRKVGVPAAMAERPFQCRICMQNFSRSDNLSVHIRTHTGEKPFACDICGRKFARRAHLNQHTKIHTGSQKPFQCRICMRNFSDQSNLRAHIRTHTGEKPFACDICGRKFARRSDLKRHTKIHTGSQKPFQCRICMRNFSQSGHLARHIRTHTGEKPFACDICGRKFAQRVALQAHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 216 - protein, 298 aa, Artificial Sequence
  source: /note=“Description of Artificial Sequence Synthetic polypeptide”
MAPKKKRKVGVPAAMAERPFQCRICMQNFSQSGNLARHIRTHTGEKPFACDICGRKFAAKWNLDAHTKIHTGSQKPFQCRICMRNFSQSGDLTRHIRTHTGEKPFACDICGRKFATQGYLRKHTKIHTHPRAPIPKPFQCRICMRNFSRSDNLSVHIRTHTGEKPFACDICGRKFAQSGDLTRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 217 - protein, 298 aa, Artificial Sequence
  source: /note=“Description of Artificial Sequence Synthetic polypeptide”
MAPKKKRKVGVPAAMAERPFQCRICMQNFSTSGNLTRHIRTHTGEKPFACDICGRKFARSELLQSHTKIHTGSQKPFQCRICMRNFSARSTRITHIRTHTGEKPFACDICGRKFAQSGSLTRHTKIHTHPRAPIPKPFQCRICMRNFSQSGSLTRHIRTHTGEKPFACDICGRKFAAKWNLDAHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 218 - protein, 298 aa, Artificial Sequence
  source: /note=“Description of Artificial Sequence Synthetic polypeptide”
MAPKKKRKVGVPAAMAERPFQCRICMQNFSTSGNLTRHIRTHTGEKPFACDICGRKFARSELLQSHTKIHTGSQKPFQCRICMQNFSARSTRITHIRTHTGEKPFACDICGRKFAQSGSLTRHTKIHTHPRAPIPKPFQCRICMRNFSQSGSLTRHIRTHTGEKPFACDICGRKFAAKWNLDAHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 219 - protein, 298 aa, Artificial Sequence
  source: /note=“Description of Artificial Sequence Synthetic polypeptide”
MAPKKKRKVGVPAAMAERPFQCRICMQNFSTSGNLTRHIRTHTGEKPFACDICGRKFARSELLQSHTKIHTGSQKPFQCRICMQNFSARSTRITHIRTHTGEKPFACDICGRKFAQSGSLTRHTKIHTHPRAPIPKPFQCRICMQNFSQSGSLTRHIRTHTGEKPFACDICGRKFAAKWNLDAHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 220 - protein, 294 aa, Artificial Sequence
  source: /note=“Description of Artificial Sequence Synthetic polypeptide”
MAPKKKRKVGVPAAMAERPFQCRICMQNFSRPYTLRLHIRTHTGEKPFACDICGRKFADNSNRIKHTKIHTGSQKPFQCRICMQNFSRSANLARHIRTHTGEKPFACDICGRKFATSGHLSRHTKIHTGSQKPFQCRICMRNFSQSGHLARHIRTHTGEKPFACDICGRKFARKWTLQGHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 221 - protein, 294 aa, Artificial Sequence
  source: /note=“Description of Artificial Sequence Synthetic polypeptide”
MAPKKKRKVGVPAAMAERPFQCRICMQNFSRPYTLRLHIRTHTGEKPFACDICGRKFADNSNRIKHTKIHTGSQKPFQCRICMQNFSRSANLARHIRTHTGEKPFACDICGRKFATSGHLSRHTKIHTGSQKPFQCRICMQNFSQSGHLARHIRTHTGEKPFACDICGRKFARKWTLQGHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 222 - protein, 294 aa, Artificial Sequence
  source: /note=“Description of Artificial Sequence Synthetic polypeptide”
MAPKKKRKVGVPAAMAERPFQCRICMQNFSQSADRTKHIRTHTGEKPFACDICGRKFADNSNRIKHTKIHTGSQKPFQCRICMQNFSRSDNLSTHIRTHTGEKPFACDICGRKFADSANRIKHTKIHTGSQKPFQCRICMQNFSRSDHLSEHIRTHTGEKPFACDICGRKFAQNANRITHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 223 - protein, 298 aa, Artificial Sequence
  source: /note=“Description of Artificial Sequence Synthetic polypeptide”
MAPKKKRKVGVPAAMAERPFQCRICMQNFSQSADRTKHIRTHTGEKPFACDICGRKFADNSNRIKHTKIHTGSQKPFQCRICMRNFSRSANLSVHIRTHTGEKPFACDICGRKFADRANLSRHTKIHTHPRAPIPKPFQCRICMRNFSRSDNLSVHIRTHTGEKPFACDICGRKFAQKATRITHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 224 - protein, 298 aa, Artificial Sequence
  source: /note=“Description of Artificial Sequence Synthetic polypeptide”
MAPKKKRKVGVPAAMAERPFQCRICMQNFSQSADRTKHIRTHTGEKPFACDICGRKFADNSNRIKHTKIHTGSQKPFQCRICMQNFSRSANLSVHIRTHTGEKPFACDICGRKFADRANLSRHTKIHTHPRAPIPKPFQCRICMRNFSRSDNLSVHIRTHTGEKPFACDICGRKFAQKATRITHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 225 - protein, 298 aa, Artificial Sequence
  source: /note=“Description of Artificial Sequence Synthetic polypeptide”
MAPKKKRKVGVPAAMAERPFQCRICMQNFSQSADRTKHIRTHTGEKPFACDICGRKFADNSNRIKHTKIHTGSQKPFQCRICMQNFSRSANLSVHIRTHTGEKPFACDICGRKFADRANLSRHTKIHTHPRAPIPKPFQCRICMQNFSRSDNLSVHIRTHTGEKPFACDICGRKFAQKATRITHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 226 - DNA, 18 bp, Homo sapiens
aaagcagcgagcgccggg

(19) United States
(12) Patent Application Publication
(10) Pub. No.: US 2024/0018203 A1
(43) Pub. Date: Jan. 18, 2024

(54) NOVEL ZINC FINGER PROTEIN TRANSCRIPTION FACTORS FOR REPRESSING ALPHA-SYNUCLEIN EXPRESSION

(71) Applicant: Sangamo Therapeutics, Inc., Brisbane, CA (US)

(72) Inventors: Asa Hatami, Pleasanton, CA (US); Bryan Zeitler, El Sobrante, CA (US); Lei Zhang, Davis, CA (US); David Emanuel Paschon, Castro Valley, CA (US)

(73) Assignee: Sangamo Therapeutics, Inc., Brisbane, CA (US)

(21) Appl. No.: 18/247,093

(22) PCT Filed: Oct. 1, 2021
(86) PCT No.: PCT/US2021/053166

§ 371 Date: Mar. 29, 2023

Related U.S. Application Data
(60) Provisional application No. 63/087,164, filed on Oct. 2, 2020.

(51) Int. Cl. C07K 14/47; C12N 15/86; A61P 25/28

(52) U.S. Cl. CPC C07K 14/4703; C12N 15/86; A61P 25/28; C07K 2319/81; C12N 2750/14143

(57) ABSTRACT

The present disclosure provides zinc finger fusion proteins that inhibit expression of alpha-synuclein in the nervous system, and methods of using the proteins to treat Parkinsons disease, Lewy body dementia, multiple system atrophy, Alzheimers disease, and other neurodegenerative diseases.

### CROSS-REFERENCE TO RELATED APPLICATION

This application claims priority from U.S. Provisional Application 63/087,164, filed Oct. 2, 2020, the disclosure of which is incorporated herein by reference in its entirety.

### SEQUENCE LISTING

The instant application contains a Sequence Listing which has been submitted electronically in ASCII format and is hereby incorporated by reference in its entirety. The electronic copy of the Sequence Listing, created on Sep. 30, 2021, is named 025297_WO035_SL.txt and is 167,361 bytes in size.

### BACKGROUND OF THE INVENTION

Parkinson's disease (PD) is a neurodegenerative disorder characterized by motor deficits. Approximately 50% of PD patients eventually develop dementia. PD is the second most prevalent neurodegenerative disease after Alzheimer's disease. In the United States, there are approximately one million PD patients and every year there are 50,000-60,000 new cases. Sporadic forms of PD have a typical onset age of 60-70 years. There is generally a span of 15-20 years from initial diagnosis to death from PD complications.

PD patients display a range of motoric symptoms such as bradykinesia, rigidity, stooped posture, masked facial expression, forward tilt of trunk, reduced arm swinging, flexed elbows, wrists, hips and knees, postural instability, tremor of extremities at rest, and shuffling, short-stepped gait. The patients often also have non-motoric symptoms, including anosmia, disordered sleep, reduced gut motility, neuropathic pain, and dementia. See, e.g., Jeanjean and Aubert, Lancet (2011) 378(9805):1773-4; Kalia and Lang, Lancet (2015) 386(9996):896-912.

The brains of PD patients are characterized by the loss of dopamine-producing (dopaminergic) neurons in an area called the substantia nigra. PD patients' brains also are characterized by the presence of Lewy bodies, which are protein aggregates or clumps formed inside neurons, and Lewy neurites, which are neurites (processes of neurons) containing protein aggregates similar to Lewy bodies. Lewy bodies were first discovered in the brains of PD patients by Friedrich Lewy in 1912 and were later found to contain fibrils of aggregated and insoluble forms of alpha-synuclein (Goedert and Spillantini, Mol Psychiatry. (1998) 3(6):462-5; Spillantini et al., Neurosci Lett. (1998) 251(3):205-8; Spillantini et al., Nature (1997) 388(6645):839-40). A mutation in the alpha-synuclein gene (SNCA) was identified in families with PD in 1997 (see, e.g., Polymeropoulos et al., Science (1997) 276(5321):2045-7). Later, duplication and triplication of the SNCA gene, as well as additional point mutations in alpha-synuclein, were shown to correlate with the genetic or familial forms of PD. Moreover, changes in parts of the genome that control the level of alpha-synuclein expression have been shown to be associated with an increased risk for PD in large unbiased population studies (genome-wide association studies, GWAS).

Alpha-synuclein is a membrane-bound protein involved in vesicle release at presynaptic terminals of neurons. It may also play a role in DNA repair. Mature alpha-synuclein is a small 14-kD protein with a central core region (residues 61-95) containing hydrophobic amino acids, known as the NAC (non-A-beta component of Alzheimer's disease amyloid) region. The NAC contributes to protein aggregation. Misfolded alpha-synuclein polypeptides aggregate into oligomers and protofibrils, which then come together to form the large, insoluble aggregates found in Lewy bodies. Accumulating evidence indicates that alpha-synuclein misfolding and aggregation play a central role in the cellular damage that occurs in PD and eventually lead to the death of neurons in the substantia nigra. Furthermore, smaller aggregates of alpha-synuclein have been shown to move from cell to cell and spread throughout the brain, similar to what is seen in prion diseases. Inhibition of alpha-synuclein aggregation may reduce damage to neurons and slow down or even halt the progression of PD.

Current approaches to reduce the levels of alpha-synuclein include the use of anti-sense oligonucleotides (ASOs), which target alpha-synuclein at the RNA level, and monoclonal antibodies (mAbs), which target specific 3D shapes or conformations of alpha-synuclein outside cells. However, there remains an urgent need for a clinically efficacious method of treating PD by targeting alpha-synuclein.

### SUMMARY OF THE INVENTION

The present disclosure provides zinc finger protein (ZFP) domains that target sites in or near the human SNCA gene. The ZFP domains of the present disclosure may be fused to a transcription factor to specifically inhibit expression of the human SNCA gene at the DNA level. These fusion proteins contain (i) a ZFP domain that binds specifically to a target region in the SNCA gene and (ii) a transcription repressor domain that reduces the transcription of the gene.

In one aspect, the present disclosure provides a fusion protein comprising a zinc finger protein (ZFP) domain and a transcription repressor domain, wherein the ZFP domain binds to a target region of a human alpha-synuclein gene (SNCA gene). In some embodiments, the target region (i.e., target site) is within about 1 kb of a transcription start site (TSS, e.g., TSS 1, 2a, or 2b) in the SNCA gene. In further embodiments, the target region is within about 500 bps upstream of TSS 2a, within about 500 bps downstream of TSS 2b, and/or within about 500 bps upstream or downstream of TSS 1 of the SNCA gene as shown in FIGS. 2B and/or 4. Nonlimiting examples of target regions are shown in Table 1.

In some embodiments, the fusion protein comprises one or more (e.g., two, three, four, five, or six) zinc fingers. It may repress expression of the SNCA gene by at least about 40%, 75%, 90%, 95%, or 99%, preferably with no or minimal detectable off-target binding or activity (e.g., binding to a gene that is not the SNCA gene). Nonlimiting examples of zinc finger domains are shown in Table 1. In some embodiments, the fusion protein comprises one or more recognition helix sequences shown in Table 1. In further embodiments, the fusion protein comprises some or all the recognition helix sequences from a single row of the table, with or without the indicated backbone mutation(s). In certain embodiments, the fusion protein comprises an amino acid sequence shown in Table 2.

In some embodiments, the transcription repressor domain of the fusion protein is from the KRAB domain of the KOX1 protein. The zinc finger domain may be linked to the transcription repressor domain through a peptide linker. In another aspect, the present disclosure provides a nucleic acid construct comprising a coding sequence for the present fusion protein, wherein the coding sequence is linked operably to a transcription regulatory element, such as a mammalian promoter that is constitutively active or inducible in a brain cell, and wherein the promoter is optionally a human synapsin I promoter. The present disclosure also provides a host cell comprising the nucleic acid construct. The host cell may be a human cell, such as a brain cell or a pluripotent stem cell, wherein the stem cell is optionally an embryonic stem cell or an inducible pluripotent stem cell (iPSC).

In another aspect, the present disclosure provides a method of inhibiting expression of alpha-synuclein in a human brain cell, comprising introducing into the cell the present fusion protein (e.g., through introduction of a nucleic acid construct or a recombinant virus such as AAV (e.g., AAV2, AAV6, AAV9, or hybrids thereof), thereby inhibiting the expression of alpha-synuclein in the cell. The brain cell may be a neuron, a glial cell, an ependymal cell, or a neuroepithelial cell. The cell may be in the brain of a patient suffering from or at risk of developing Parkinson's disease, Lewy body dementia, Alzheimer's disease, multiple system atrophy, or other synucleinopathy.

The present disclosure also provides a method of treating (e.g., slowing the progression) of a synucleinopathy in a patient, comprising administering to the patient a recombinant AAV encoding a fusion protein of the present disclosure. In some embodiments, the AAV is introduced to the patient via intravenous, intrathecal, intracerebroventricular, intra-cisternal magna, intrastriatal, or intranigral injection, or injection into any brain region. The patient may have Parkinson's disease, Lewy body dementia, Alzheimer's disease, or multiple system atrophy.

The present disclosure also provides fusion proteins for use in the above methods and use of the present fusion proteins in the manufacture of a medicament for use in the above methods.

Other features, objectives, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE DRAWINGS

FIG. 1 illustrates specific targeting of the SNCA gene by an engineered 6-finger zinc finger protein-transcription factor (ZFP-TF), which recognizes 18 base pairs in the gene. Binding of the ZFP-TF to the gene leads to reduced SNCA transcription, which in turn leads to reduced SNCA mRNA and alpha-synuclein protein levels. The figure discloses the genomic target sequence (bound sequence; SEQ ID NO: 226) for an exemplary engineered ZFP (SBS-82110).

FIG. 2A is a diagram illustrating the genomic structure of the human SNCA gene. The gene has 7 exons (2 non-protein-coding and 5 protein-coding), with each transcript containing 5 introns. There are three transcription start sites (TSSs), named TSS 1, TSS 2a, and TSS 2b, which give rise to transcripts starting with non-coding exons 1, 2a, and 2b, respectively. The first protein-coding exon in all transcripts is exon 3.

FIG. 2B is a diagram illustrating an upstream genomic region of the human SNCA gene. The SNCA mRNA sequence is shown as a red bar. The small triangles in the clusters underneath the gene depict the regions in the SNCA gene targeted by the 416 representative ZFP-TFs exemplified herein. The figure also shows the effect of each of the ZFP-TFs on reducing human SNCA mRNA expression in SK-N-MC human neuroblastoma cells harvested 24 hours after transfection with ZFP-TF mRNA. Messenger RNA levels were measured by RT-qPCR. Normalized SNCA expression levels are indicated by the gradient bar “SNCA mRNA.” Deepest (red) color denotes 100% reduction. Lightest color (white) denotes 0% reduction. RT-qPCR data were normalized to the mean of mRNA levels of two housekeeping genes (ATP5B and EIF4A2). Triangles pointing to the right indicate that the ZFP-TFs bind the sense strand of the gene. Triangles pointing to the left indicate that the ZFP-TFs bind the antisense strand of the gene.

FIGS. 3A-3E are graphs showing the screening results from a library of 416 ZFP-TFs as described herein. The screening was done in the SK-N-MC human neuroepithelial cell line. The y-axis in each graph is alpha-synuclein mRNA expression normalized to the geometric mean of two housekeeping genes (EIF4A2 and ATP5B) and assessed 24 hours after transfection with RNA coding for the different ZFP-TFs. The RNA dose increases from left to right (3, 10, 30, 100, 300, and 1,000 ng). The bars represent the mean of four technical replicates and the error bars represent standard deviation. The numbers below the graphs are the internal reference numbers for the ZFP-TFs.

FIG. 4 is a diagram illustrating an upstream genomic region of the human SNCA gene. The SNCA mRNA sequence is shown as a red bar. The small triangles in the clusters underneath the gene depict the regions in the SNCA gene targeted by 27 representative ZFP-TFs with 1, 2, or 3 phosphate contact mutations. The figure also shows the effect of each of the ZFP-TFs on reducing human SNCA mRNA expression in SK-N-MC human neuroblastoma cells harvested 24 hours after transfection with ZFP-TF mRNA. Messenger RNA levels were measured by RT-qPCR. Normalized SNCA expression levels are indicated by the gradient bar “SNCA mRNA.” Deepest (red) color denotes 100% reduction. Lightest color (white) denotes 0% reduction. RT-qPCR data were normalized to the mean of mRNA levels of two housekeeping genes (ATP5B and EIF4A2). Triangles pointing to the right indicate that the ZFP-TFs bind the sense strand of the gene. Triangles pointing to the left indicate that the ZFP-TFs bind the antisense strand of the gene.

FIG. 5 shows illustrative screening results from a library of 27 representative ZFP-TFs with 1, 2, or 3 phosphate contact mutations. The screening was done in the SK-N-MC human neuroepithelial cell line. The y-axis in each graph is alpha-synuclein mRNA expression normalized to the geometric mean of two housekeeping genes (EIF4A2 and ATP5B) and assessed 24 hours after transfection with RNA coding for the different ZFP-TFs. The RNA dose increases from left to right (3, 10, 30, 100, 300, and 1,000 ng). The bars represent the mean of four technical replicates and the error bars represent standard deviation. The numbers below the graphs are the internal reference numbers for the ZFP-TFs.

FIGS. 6A and 6B are panels of graphs showing that 40 alpha-synuclein ZFP-TFs displayed a range of alpha-synuclein repression activity in SK-N-MC human neuroblastoma cells and human iPSC-derived neurons. Twenty illustrative ZFP-TFs described in Tables 1 and 2 are shown in panel A. Illustrative ZFP-TFs characterized in FIGS. 3A-3E but not described in Tables 1 and 2 are shown in panel B. The y-axis is alpha-synuclein mRNA expression normalized to the geometric mean of two housekeeping genes (ATP5B and EIF4A2) and assessed 24 hours after transfection of SK-N-MC cells with RNA coding for the different ZFP-TFs, or 28 days after transduction of iPSC-derived neurons with AAV6 encoding the different ZFP-TFs. The amount of RNA or AAV6 used is indicated at the x-axis, with the RNA (3, 10, 30, 100, 300, and 1,000 ng) or AAV6 (1E3, 3E3, 1E4, 3E4, 1E5, and 3E5) dose increasing from left to right. The blue and orange bars represent the mean of four technical replicates and the error bars represent standard deviation. Enlarged versions of the titration scales are shown at the bottom of the figure.

FIGS. 7A-7D are panels of volcano plots depicting the off-target activity of 40 alpha-synuclein ZFP-TFs in mouse primary neurons (A and B) and human iPSC-derived neurons (C and D). For easy reference, bar graphs from FIGS. 6A-6B illustrating the repressing activity of the ZFP-TFs in human iPSC-derived neurons are shown next to the volcano plots of the corresponding ZFP-TFs. The volcano plots summarize microarray data showing changes in the transcriptome of mouse primary neurons 7 days after transduction (A and B) or human iPSC-derived neurons 19 days after transduction (C and D). Yellow circles indicate human alpha-synuclein. Green circles (right side of each volcano plot) represent genes significantly unregulated by >2-fold. The number of these genes is indicated in green text. Red circles represent genes significantly downregulated by >2-fold. The number of these genes is indicated in red text.

FIGS. 8A and 8B are panels of graphs showing (A) ZFP-TF and alpha-synuclein mRNA expression levels or (B) mRNA expression levels of glial fibrillary acidic protein (GFAP), ionized calcium binding adaptor molecule 1 (IBA1), and NeuN in different brain regions of female PAC synuclein mice (Kuo et al., Hum Mol Genet. (2010) 19(9):1633-50). The animals were bilaterally administered AAV9 coding for the indicated test article or vehicle at two sites in the striatum. All gene expression data are normalized to the geometric mean of 3 housekeeping genes (ATP5B, EIF4A2, and GAPDH). The y-axis is mRNA expression level of the indicated gene and the x-axis is brain region. The red squares indicate average expression data for n=3 animals administered ZFP-TF 82195, and the green triangles indicate average expression data for n=3 animals administered ZFP-TF 82264. In panel A, alpha-synuclein mRNA expression is normalized to the mean of 3 vehicle-treated animals, and ZFP-TF expression is provided on a log scale as copies per ng input RNA. In panel B, all expression data are normalized to the mean of the 3 vehicle-treated animals. Statistical analysis was performed using a one-way analysis of variance (ANOVA) followed by a Dunnett's multiple comparisons test. * p<0.05, ** p<0.01, ***, p<0.001, ***, p<0.0001.

### DETAILED DESCRIPTION OF THE INVENTION

The present disclosure provides ZFP domains that target sites (i.e., sequences) in or near the human SNCA gene. A ZFP domain as described herein may be attached or fused to another functional molecule or domain. The ZFP domains of the present disclosure may be fused to a transcription factor to repress transcription of the human SNCA gene into RNA. The fusion proteins are called zinc finger protein transcription factors (ZFP-TFs). These ZFP-TFs comprise a zinc finger protein (ZFP) domain that binds specifically to a target region in or near the SNCA gene and a transcription repressor domain that reduces the transcription of the gene. Reducing the level of alpha-synuclein in neurons by introducing the ZFP-TFs into the brain of a patient is expected to inhibit (e.g., reduce or stop) the assembly of alpha-synuclein into oligomeric (smaller soluble aggregates) or fibrillar (larger insoluble aggregates) forms. With a reduction in alpha-synuclein aggregation, the brain cells will have the capacity to timely remove misfolded and toxic forms of alpha-synuclein with their cellular quality control machinery. As a result, aggregation and cell-to-cell propagation of alpha-synuclein will be reduced or prevented.

Our ZFP-TF approach to alpha-synuclein inhibition has several advantages over the current approaches being tested by others. ZFP-TFs may need to be administered only once (by introducing to the patient a ZFP-TF expression construct), while ASOs require repeated dosing. In addition, the ZFP-TF approach only needs to engage the two alleles of the SNCA gene in the genome of each cell. By contrast, ASOs need to engage numerous copies of the SNCA mRNA in each cell.

Our ZFP-TF approach is advantageous over the antibody approach because antibodies can only bind a subset of alpha-synuclein shapes or conformations. This may not be sufficient for a robust therapeutic effect. In contrast, ZFP-TFs repress alpha-synuclein expression at the DNA level and lead to lower levels of all forms of alpha-synuclein. ZFP-TFs are therefore agnostic to the forms of the toxic species, unlike antibodies. In addition, antibodies are thought to largely act on alpha-synuclein on or outside the cells, whereas ZFP-TFs can reduce alpha-synuclein inside the cell directly and indirectly lower extracellular alpha-synuclein levels. Thus, the ZFP-TF approach is expected to be more effective because alpha-synuclein is largely an intracellular protein. Further, antibodies require repeated administration, while ZFP-TFs require only a one-time delivery of their expression constructs.

### I. Targets of the ZFP Domains

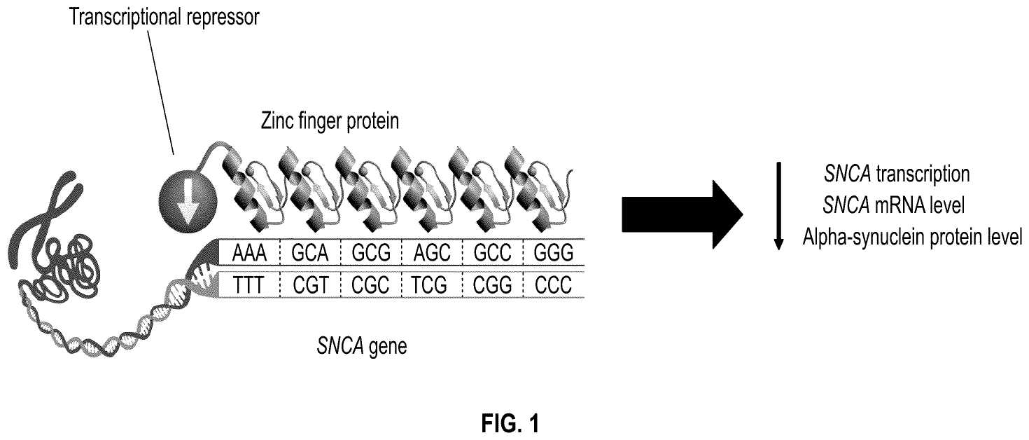

The ZFP domains of the present fusion proteins bind specifically to a target region in or near the human SNCA gene. FIG. 1 illustrates the binding of a ZFP domain to a target SNCA gene sequence. The ZFP domain in the figure has six zinc fingers; however, as further described below, a ZFP domain that has fewer or more zinc fingers can also be used.

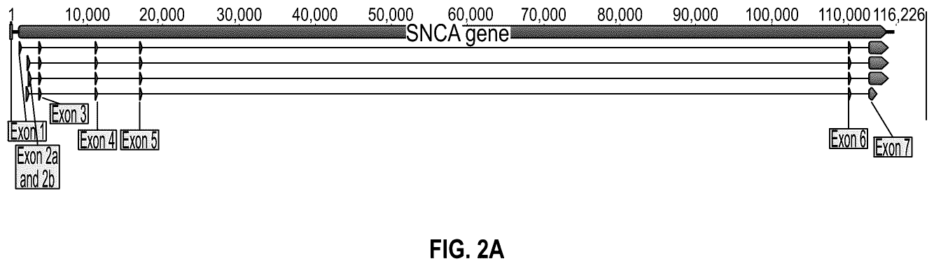

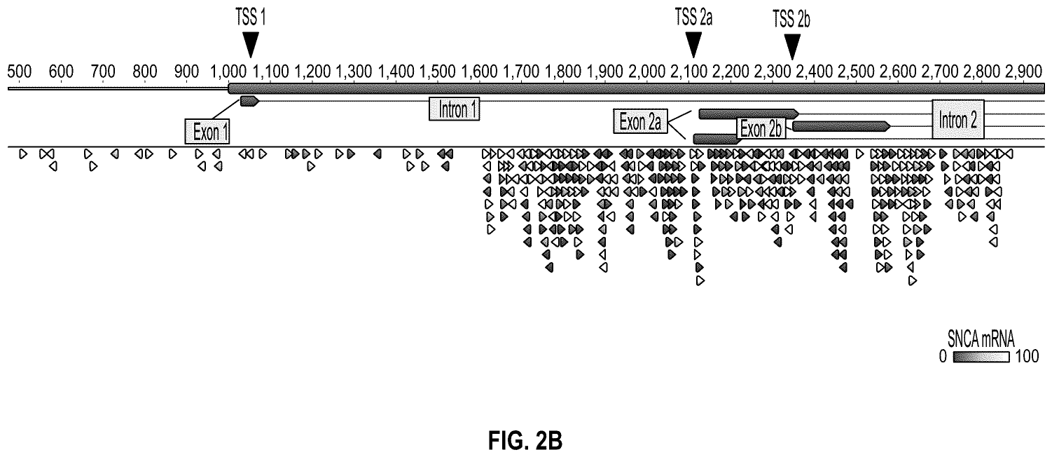

The human SNCA gene spans about 117 kb and has been mapped to chr4:89,724,099-89,838,315(GRCh38/hg38). Its nucleotide sequence is available at GenBank accession number NC_000004 version 000004.12. The gene has 7 exons (2 non-protein-coding, and 5 protein-coding), with each transcript having 5 introns (FIG. 2A). There are three transcription start sites (TSSs) for the gene: one at the beginning of exon 1, and two in exon 2, resulting in the expression of exons 2a and 2b. The first protein-coding exon is exon 3. See also Touchman et al., Genome Res. (2001) 11:78-86. Isoform 1 (full length) of human alpha-synuclein is shown below:

(SEQ ID NO: 1; P37840-1)
MDVFMKGLSK AKEGVVAAAE KTKQGVAEAA GKTKEGVLYV
GSKTKEGVVH GVATVAEKTK EQVTNVGGAV VTGVTAVAQK
TVEGAGSIAA ATGFVKKDQL GKNEEGAPQE GILEDMPVDP
DNEAYEMPSE EGYQDYEPEA

Isoform 2-4 differs from isoform 1 in that amino acid residues 103-130 are missing. Isoform 2-5 differs from isoform 1 in that amino acid residues 41-54 are missing. Genetic analysis of alpha-synuclein has pointed to gene copy amplification (see, e.g., Brueggemann et al., Neurology (2008) 71:1294; Troiano et al., Neurology (2008) 71:1295; Uchiyama et al., Neurology (2008) 71:1289-90) and certain point mutations as potential causes for synucleinopathies such as PD and Lewy body dementia. For example, the following alpha-synuclein point mutations have been identified in some PD patients: A30P (Kruger et al., Nature Genet. (1998) 18:106-8); E46K (Zarranz et al., Ann Neurol. (2004) 55:164-73; Choi et al., FEBS Lett. (2004) 576:363-8); H50Q (Khalaf et al., J Biol Chem. (2014) 289:21856-76); G51D (Lesage et al., Ann Neurol. (2013) 73:459-71); and A53T (Polymeropoulos et al., Science (1997) 276:2045-7).

The DNA-binding ZFP domain of the ZFP-TFs directs the fusion proteins to a target region of the SNCA gene and brings the transcription repressor domain of the fusion proteins to the target region. The repressor domain then represses the SNCA gene's transcription by RNA polymerase. The target region for the ZFP-TFs can be any suitable site in or near the SNCA gene that allows repression of gene expression. By way of example, the target region includes, or is adjacent to (either downstream or upstream of) an SNCA TSS or an SNCA transcription regulatory element (e.g., promoter, enhancer, RNA polymerase pause site, and the like).

As described above, the human SNCA gene has three transcription start sites (TSSs). They are, from 5′ to 3′, TSS 1, TSS 2a, and TSS 2b, which are located at the 5′ ends of exon 1 (TSS 1), exon 2a, and exon 2b (TSSs 2a and 2b) (FIG. 2B). Transcription at the three TSSs leads to RNA isoforms with different lengths. However, since translation of alpha-synuclein begins at exon 3, RNA produced from the different TSSs leads to the formation of the same protein. In some embodiments, the genomic target region of the present ZFP-TFs spans or is in the vicinity of TSS 1 (e.g., base pairs 529-1529) or TSSs 2a and 2b (e.g., base pairs 1613-2949). In certain embodiments, the target region is within about 500 bp upstream or downstream of TSS 1, and/or within about 500 bp upstream or downstream of TSS 2a, and/or within about 500 bp upstream or downstream of TSS 2b.

In some embodiments, the genomic target region is at least 8 bps in length. For example, the target region may be 8 bps to 40 bps in length, such as 12, 15, 18, 21, 24, 27, 30, 33, or 36 bps in length. The targeted sequence may be on the sense strand of the gene, or the antisense strand of the gene. To ensure targeting accuracy and to reduce off-target binding or activity by the ZFP-TFs, the sequence of the selected SNCA target region preferably has less than 75% homology (e.g., less than 70%, less than 65%, less than 60%, or less than 50%) to sequences in other genes. In certain embodiments, the target region of the present ZFP-TFs is 15-18 bps in length and resides within 500 bps of TSSs 1, 2a, or 2b. Examples of target regions are shown in FIG. 2B and Table 1.

In some embodiments, the present engineered ZFPs bind to a target site (i.e., Binding Sequence) as shown in a single row of Table 1, preferably with no or little detectable off-target binding or activity.

Other criteria for further evaluating target segments include the prior availability of ZFPs binding to such segments or related segments, ease of designing new ZFPs to bind a given target segment, and off-target binding risk.

### II. ZFP Domains

A “zinc finger protein” or “ZFP” refers to a protein having a DNA-binding domain that is stabilized by zinc. ZFPs bind to DNA in a sequence-specific manner. The individual DNA-binding unit of a ZFP is referred to as a zinc “finger.” Each finger contains a DNA-binding “recognition helix” that is typically comprised of seven amino acid residues and determines DNA binding specificity. A ZFP domain has at least one finger, each finger binds from two to four base pairs of DNA, typically three or four base pairs of DNA. Each zinc finger typically comprises approximately 30 amino acids and chelates zinc. An engineered ZFP can have a novel binding specificity, compared to a naturally occurring ZFP. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers that bind the particular triplet or quadruplet sequence. See, e.g., ZFP design methods described in detail in U.S. Pat. Nos. 5,789,538; 5,925,523; 6,007,988; 6,013,453; 6,140,081; 6,200,759; 6,453,242; 6,534,261; 6,979,539; and 8,586,526; and International Patent Publications WO 95/19431; WO 96/06166; WO 98/53057; WO 98/53058; WO 98/53059; WO 98/53060; WO 98/54311; WO 00/27878; WO 01/60970; WO 01/88197; WO 02/016536; WO 02/099084; and WO 03/016496. A ZFP domain as described herein may be attached or fused to another molecule, for example, a protein. Such ZFP-fusions may comprise a domain that enables gene activation (e.g., activation domain), gene repression (e.g., repression domain), ligand binding (e.g., ligand-binding domain), high-throughput screening (e.g., ligand-binding domain), localized hypermutation (e.g., activation-induced cytidine deaminase domain), chromatin modification (e.g., histone deacetylase domain), recombination (e.g., recombinase domain), targeted integration (e.g., integrase domain), DNA modification (e.g., DNA methyl-transferase domain), base editing (e.g., base editor domain), or targeted DNA cleavage (e.g., nuclease domain). Examples of engineered ZFP domains are shown in Table 1.

The ZFP domain of the present engineered ZFP fusion proteins may include at least one (e.g., one, two, three, four, five, six, seven, eight, nine, ten, eleven, twelve, thirteen, or more) zinc finger(s). A ZFP domain having one finger typically recognizes a target site that includes 3 or 4 nucleotides. A ZFP domain having two fingers typically recognizes a target site that includes 6 or 8 nucleotides. A ZFP domain having three fingers typically recognizes a target site that includes 9 or 12 nucleotides. A ZFP domain having four fingers typically recognizes a target site that includes 12 to 15 nucleotides. A ZFP domain having five fingers typically recognizes a target site that includes 15 to 18 nucleotides. A ZFP domain having six fingers can recognize target sites that include 18 to 21 nucleotides.

In some embodiments, the present engineered ZFPs comprise a DNA-binding recognition helix sequence shown in Table 1. For example, an engineered ZFP may comprise the sequence of F1, F2, F3, F4, F5, or F6 as shown in Table 1.

In some embodiments, the present engineered ZFPs comprise two adjacent DNA-binding recognition helix sequences shown in a single row of Table 1. For example, an engineered ZFP may comprise the sequences of F1-F2, F2-F3, F3-F4, F4-F5, or F5-F6 as shown in a single row of Table 1.

In some embodiments, the present engineered ZFPs comprise the DNA-binding recognition helix sequences shown in a single row of Table 1. For example, an engineered ZFP may comprise the sequences of F1, F2, F3, F4, F5, and F6 (e.g., F1-F6) as shown in a single row of Table 1.

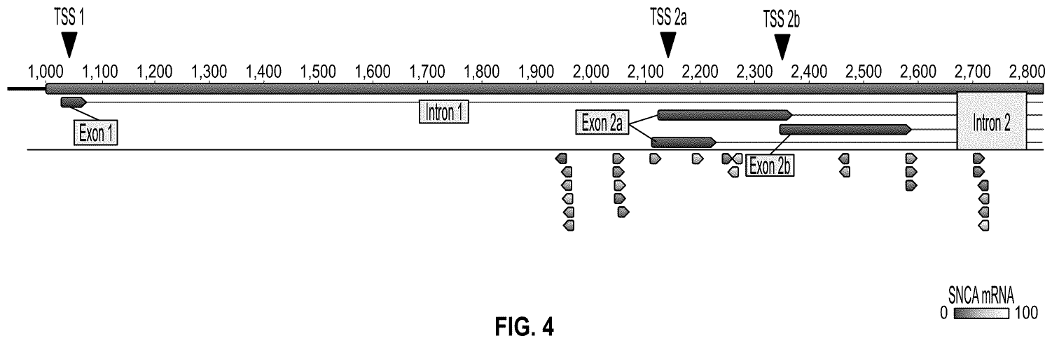

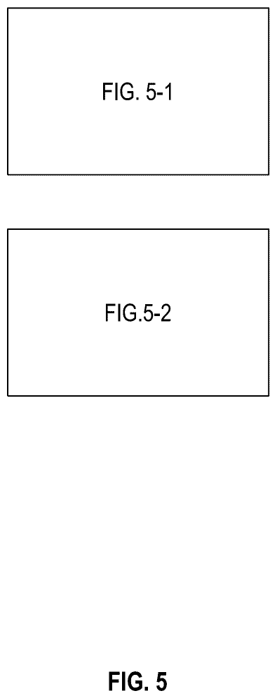

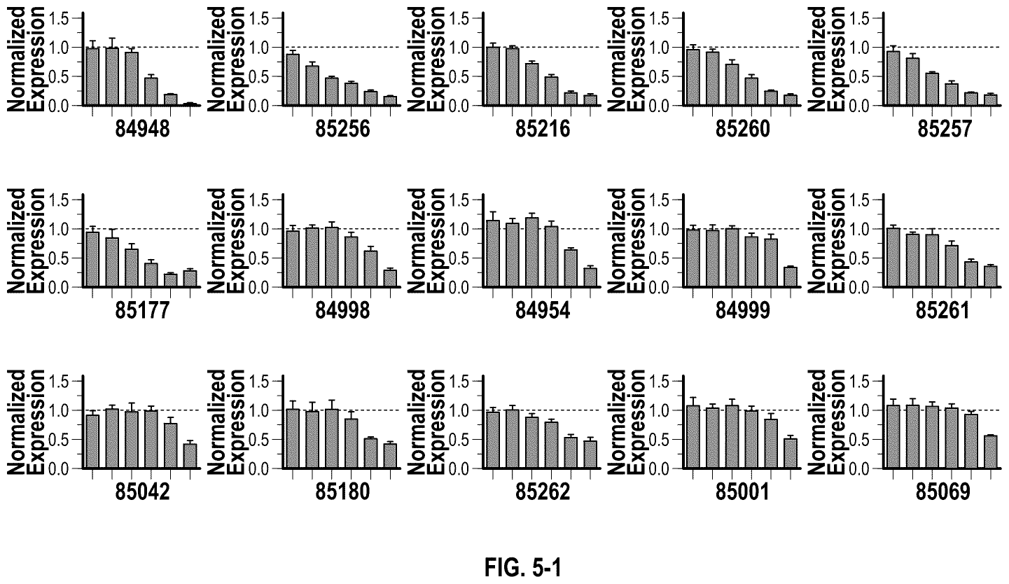

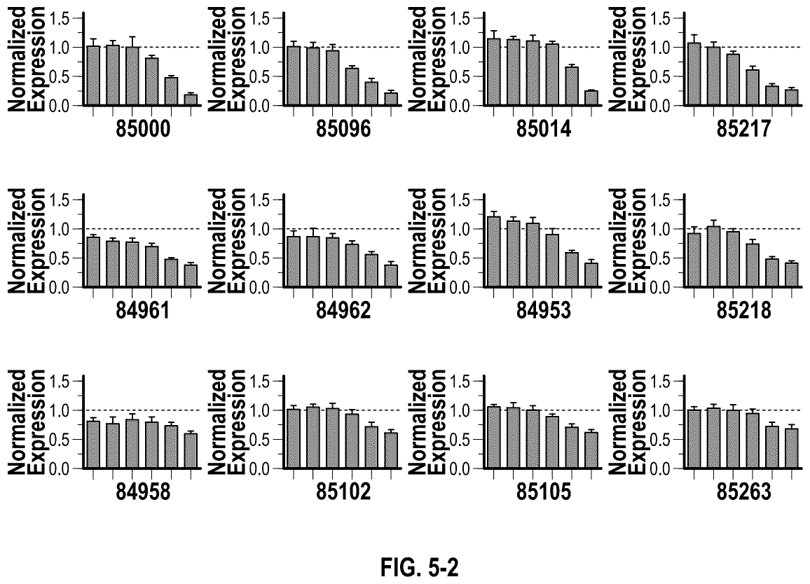

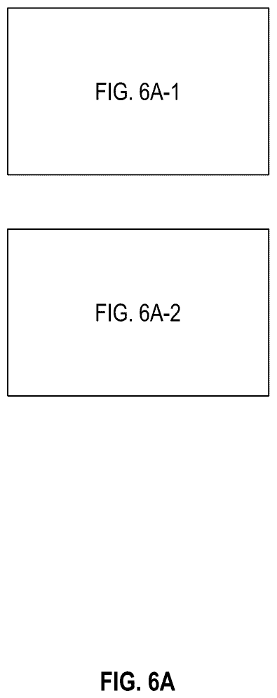

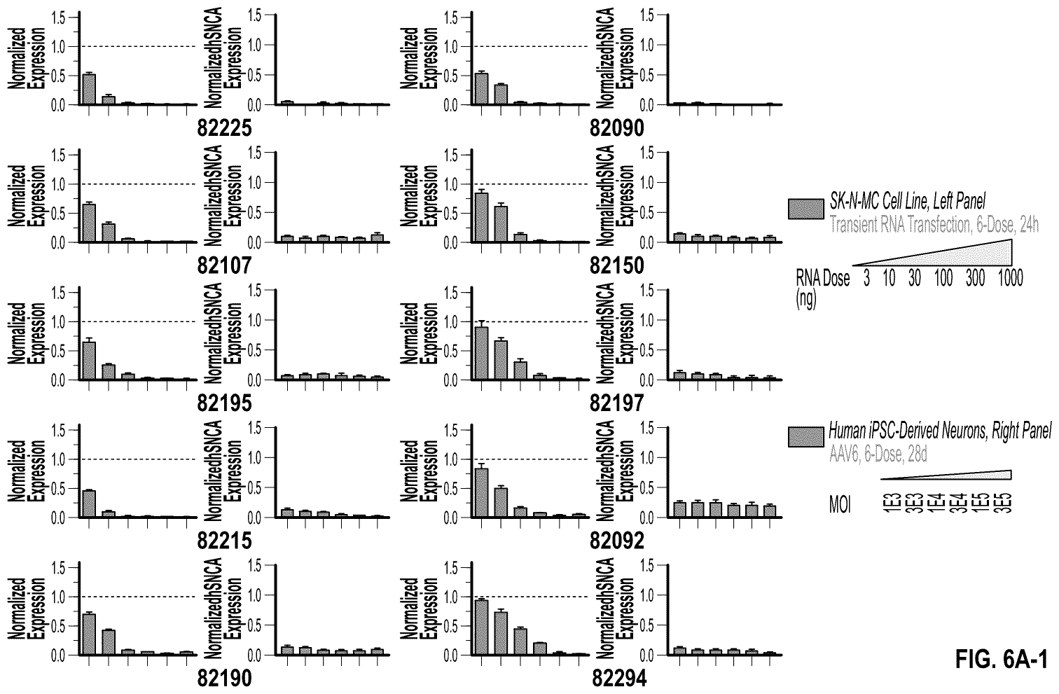

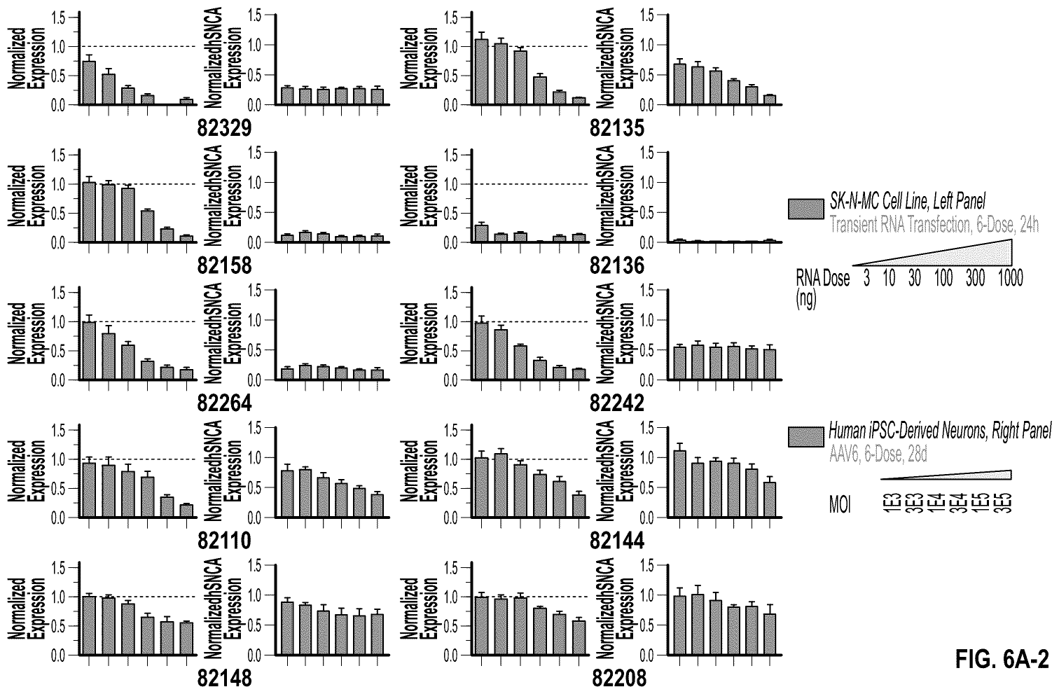

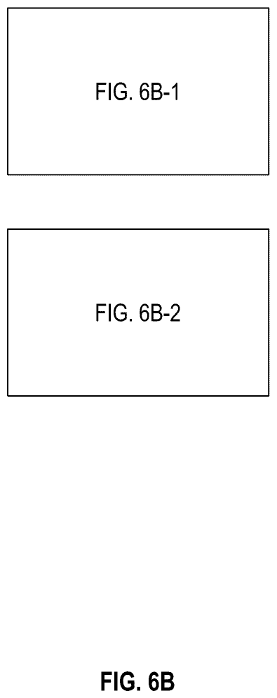

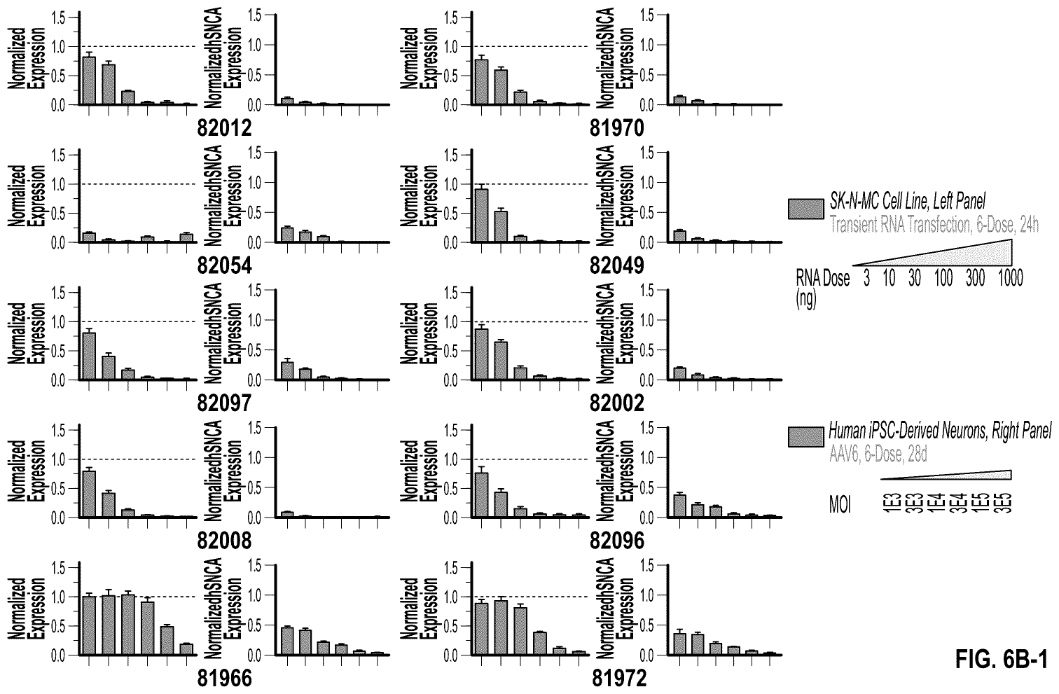

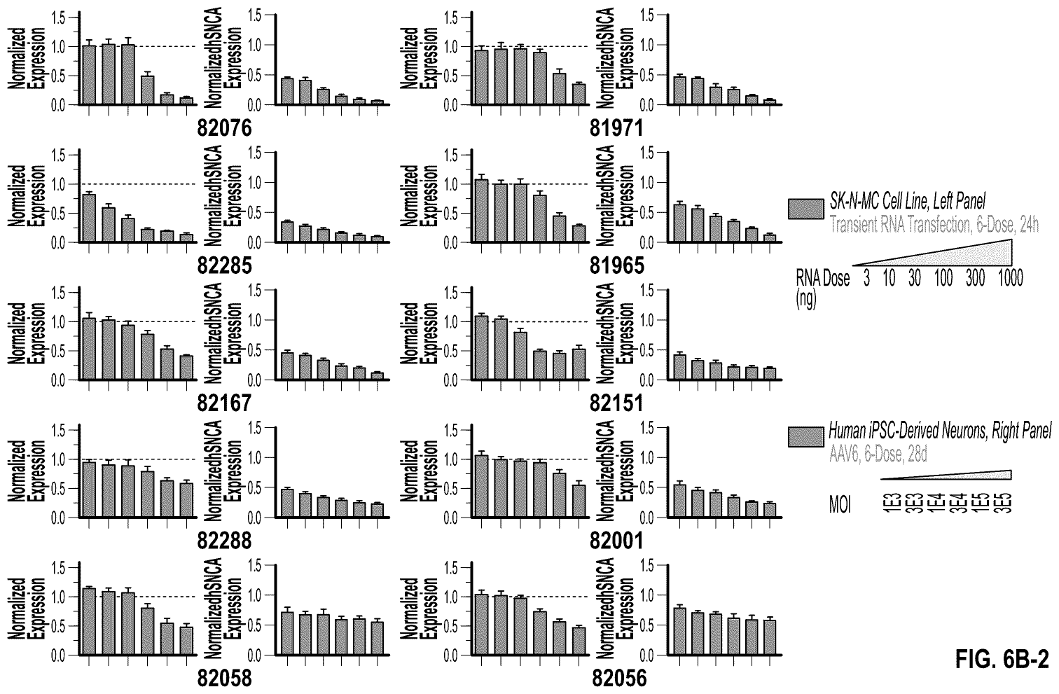

The target specificity of the ZFP domain may be improved by mutations to the ZFP backbone sequence as described in, e.g., U.S. Pat. Pub. 2018/0087072. The mutations include those made to residues in the ZFP backbone that can interact non-specifically with phosphates on the DNA backbone but are not involved in nucleotide target specificity. In some embodiments, these mutations comprise mutating a cationic amino acid residue to a neutral or anionic amino acid residue. In some embodiments, these mutations comprise mutating a polar amino acid residue to a neutral or non-polar amino acid residue. In further embodiments, mutations are made at positions (−5), (−9) and/or (−14) relative to the DNA binding helix. In some embodiments, a zinc finger may comprise one or more mutations at positions (−5), (−9) and/or (−14). In further embodiments, one or more zinc fingers in a multi-finger ZFP domain may comprise mutations at positions (−5), (−9) and/or (−14). In some embodiments, the amino acids at positions (−5), (−9) and/or (−14) (e.g., an arginine (R) or lysine (K)) are mutated to an alanine (A), leucine (L), serine (S), aspartate (N), glutamate (E), tyrosine (Y), and/or glutamine (Q). Examples of engineered ZFPs with 1, 2, or 3 backbone mutations are shown in FIGS. 4 and 5 and Tables 1 and 2. The symbol “\hat{ }” in Table 1 indicates that the arginine (R) residue at the 4th position upstream of the 1st amino acid in the indicated recognition helix is changed to glutamine (Q). In each recognition helix sequence, the positions of the seven DNA-binding amino acids are numbered −1, +1, +2, +3, +4, +5, and +6. Thus, the position for the R-to-Q substitution is numbered as (−5).

In some embodiments, the present engineered ZFPs comprise a DNA-binding recognition helix sequence and associated backbone mutation as shown in Table 1. In some embodiments, the present engineered ZFPs comprise the DNA-binding recognition helix sequences and associated backbone mutations as shown in a single row of Table 1.

In some embodiments, an engineered ZFP described herein comprises the recognition helix and backbone portions of a sequence shown in a single row of Table 2. In some embodiments, an engineered ZFP described herein comprises the recognition helix and backbone portions of a sequence shown in a single row of Table 2 as the sequence would appear following post-translational modification. For example, post-translational modification may remove the initiator methionine residue from a sequence as shown in Table 2.

In some embodiments, the present ZFP-TFs comprise one or more zinc finger domains. The domains may be linked together via an extendable flexible linker such that, for example, one domain comprises one or more (e.g., 4, 5, or 6) zinc fingers and another domain comprises additional one or more (e.g., 4, 5, or 6) zinc fingers. In some embodiments, the linker is a standard inter-finger linker such that the finger array comprises one DNA binding domain comprising 8, 9, 10, 11 or 12 or more fingers. In other embodiments, the linker is an atypical linker such as a flexible linker. For example, two ZFP domains may be linked to a transcription repressor TF in the configuration (from N terminus to C terminus) ZFP-ZFP-TF, TF-ZFP-ZFP, ZFP-TF-ZFP, or ZFP-TF-ZFP-TF (two ZFP-TF fusion proteins are fused together via a linker).

In some embodiments, the ZFP-TFs are “two-handed,” i.e., they contain two zinc finger clusters (two ZFP domains) separated by intervening amino acids so that the two ZFP domains bind to two discontinuous target sites. An example of a two-handed type of zinc finger binding protein is SIP1, where a cluster of four zinc fingers is located at the amino terminus of the protein and a cluster of three fingers is located at the carboxyl terminus (see Remade et al., EMBO J. (1999) 18(18):5073-84). Each cluster of zinc fingers in these proteins is able to bind to a unique target sequence and the spacing between the two target sequences can comprise many nucleotides.

### III. Zinc Finger Protein Transcription Factors

The ZFP domains described herein may be fused to a transcription factor. In some embodiments, the present fusion proteins contain a DNA-binding zinc finger protein (ZFP) domain and a transcription factor domain (i.e., ZFP-TF). In some embodiments, the transcription factor may be a transcription repressor domain, wherein the ZFP and repressor domains may be associated with each other by a direct peptidyl linkage or a peptide linker, or by dimerization (e.g., through a leucine zipper, a STAT protein N terminal domain, or an FK506 binding protein). As used herein, a “fusion protein” refers to a polypeptide with covalently linked domains as well as a complex of polypeptides associated with each other through non-covalent bonds. The transcription repressor domain can be associated with the ZFP domain at any suitable position, including the C- or N-terminus of the ZFP domain.

In some embodiments, the present ZFP-TFs bind to their target with a K_{D} of less than about 25 nM and repress transcription of a human SNCA gene by 20% or more (e.g., by 30%, 40%, 50%, 60%, 70%, 80%, 90%, or 95% or more). In some embodiments, two or more of the present ZFP-TFs are used concurrently in a patient, where the ZFP-TFs bind to different target regions in the SNCA gene, so as to achieve optimal repression of SNCA expression.

A. Transcription Repressor Domains

The present ZFP-TFs comprise an engineered ZFP domain as described herein and one or more transcription repressor domains that dampen the transcription activity of the SNCA gene. One or more engineered ZFP domains and one or more transcription repressor domains may be joined by a flexible linker. Non-limiting examples of transcription repressor domains are KRAB domain of KOX1, KAP-1, MAD, FKHR, EGR-1, ERD, SID, TGF-beta-inducible early gene (TIEG), v-ERB-A, MBD2, MBD3, TRa, histone methyltransferase, histone deacetylase (HDAC), nuclear hormone receptor (e.g., estrogen receptor or thyroid hormone receptor), members of the DNMT family (e.g., DNMT1, DNMT3A, DNMT3B), Rb, and MeCP2. See, e.g., Bird et al. (1999) Cell 99:451-454; Tyler et al. (1999) Cell 99:443-446; Knoepfler et al. (1999) Cell 99:447-450; and Robertson et al. (2000) Nature Genet. 25:338-342. Additional exemplary repression domains include, but are not limited to, ROM2 and AtHD2A. See, for example, Chem et al. (1996) Plant Cell 8:305-321; and Wu et al. (2000) Plant J. 22:19-27.

In some embodiments, the transcription repressor domain comprises a sequence from the Kruppel-associated box (KRAB) domain of the human zinc finger protein 10/KOX1 (ZNF10/KOX1) (e.g., GenBank No. NM 015394.4). An exemplary KRAB domain sequence is:

(SEQ ID NO: 12)
DAKSLTAWSR TLVTFKDVFV DFTREEWKLL DTAQQIVYRN
VMLENYKNLV SLGYQLTKPD VILRLEKGEE PWLVEREIHQ
ETHPDSETAF EIKSSV

Variants of this KRAB sequence may also be used so long as they have the same or similar transcription repressor function.

In some embodiments, an engineered ZFP-TF described herein binds to a target site as shown in a single row of Table 1, preferably with no or little detectable off-target binding or activity. Off-target binding may be determined, for example, by measuring the activity of ZFP-TFs at off-target genes. In some embodiments, an engineered ZFP-TF described herein comprises a DNA-binding recognition helix sequence shown in Table 1. In some embodiments, an engineered ZFP-TF described herein comprises two adjacent DNA-binding recognition helix sequences shown in a single row of Table 1. In some embodiments, an engineered ZFP-TF described herein comprises the DNA-binding recognition helix sequences shown in a single row of Table 1. In some embodiments, an engineered ZFP-TF described herein comprises the recognition helix and backbone portions of a sequence shown in a single row of Table 2. In some embodiments, an engineered ZFP-TF described herein comprises an amino acid sequence as shown in a single row of Table 2. In some embodiments, an engineered ZFP-TF described herein comprises the recognition helix and backbone portions of a sequence shown in a single row of Table 2 as the sequence would appear following post-translational modification. In some embodiments, an engineered ZFP-TF described herein comprises an amino acid sequence as shown in a single row of Table 2 as the sequence would appear following post-translational modification. For example, post-translational modification may remove the initiator methionine residue from a sequence as shown in Table 2.

B. Peptide Linkers

The ZFP domain and the transcription repressor domain of the present ZFP-TFs and/or the zinc fingers within the ZFP domains may be linked through a peptide linker, e.g., a noncleavable peptide linker of about 5 to 200 amino acids (e.g., 5, 6, 7, 8, 9, 10, 11, 12, 13, 14, 15, 16, 17, 18, 19, 20 or more amino acids). Preferred linkers are typically flexible amino acid subsequences that are synthesized as a recombinant fusion protein. See, e.g., description above; and U.S. Pat. Nos. 6,479,626; 6,903,185; 7,153,949; 8,772,453; and 9,163,245; and WO 2011/139349. The proteins described herein may include any combination of suitable linkers. Non-limiting examples of linkers are DGGGS (SEQ ID NO: 2), TGEKP (SEQ ID NO: 3), LRQKDGERP (SEQ ID NO: 4), GGRR (SEQ ID NO: 5), GGRRGGGS (SEQ ID NO: 6), LRQRDGERP (SEQ ID NO: 7), LRQKDGGGSERP (SEQ ID NO: 8), LRQKD(G3S)₂ ERP (SEQ ID NO: 9), and TGSQKP (SEQ ID NO: 10).

In some embodiments, TGEKPFA (SEQ ID NO: 15) and/or TGSQKPFQ (SEQ ID NO: 16) links the zinc fingers within the ZFP domain, and/or LRQKDAARGSGG (SEQ ID NO: 17) or LRGSGG (SEQ ID NO: 18) links the ZFP domain to the transcription repressor domain.

In some embodiments, the peptide linker is three to 20 amino acid residues in length and is rich in G and/or S. Non-limiting examples of such linkers are G4S-type linkers (SEQ ID NO: 11), i.e., linkers containing one or more (e.g., 2, 3, or 4) GGGGS (SEQ ID NO: 11) motifs, or variations of the motif (such as ones that have one, two, or three amino acid insertions, deletions, and substitutions from the motif).

### IV. Expression of the ZFP-TFs

A ZFP-TF of the present disclosure may be introduced to a patient through a nucleic acid molecule encoding it. The nucleic acid molecule may be an RNA or cDNA molecule. The nucleic acid molecule may be introduced into the brain of the patient through injection of a composition comprising a lipid:nucleic acid complex (e.g., a liposome). Alternatively, the ZFP-TF may be introduced to the patient through a nucleic acid expression vector comprising a sequence encoding the ZFP-TF. The expression vectors may include expression control sequences such as promoters, enhancers, transcription signal sequences, and transcription termination sequences that allow expression of the coding sequence for the ZFP-TFs in the cells of the nervous system. In some embodiments, the expression vector remains present in the cell as a stable episome. In other embodiments, the expression vector is integrated into the genome of the cell.

In some embodiments, the promoter on the vector for directing the ZFP-TF expression in the brain is a constitutive active promoter or an inducible promoter. Suitable promoters include, without limitation, a retroviral RSV LTR promoter (optionally with an RSV enhancer), a CMV promoter (optionally with a CMV enhancer), a CMV immediate early promoter, an SV40 promoter, a dihydrofolate reductase (DHFR) promoter, a β-actin promoter, a phosphoglycerate kinase (PGK) promoter, an EF1α promoter, a MoMLV LTR promoter, a CK6 promoter, a transthyretin promoter (TTR), a TK promoter, a tetracycline responsive promoter (TRE), an HBV promoter, an hAAT promoter, chimeric liver-specific promoters (LSPs), an E2F promoter, the telomerase (hTERT) promoter, a CMV enhancer/chicken β-actin/rabbit β-globin promoter (CAG promoter; Niwa et al., Gene (1991) 108(2):193-9), and an RU-486-responsive promoter. Brain cell-specific promoters such as a synapsin I promoter, a MeCP2 promoter, a CAMKII promoter, a PrP promoter, a GFAP promoter, or an engineered or natural promoter that restricts expression to neurons and/or glial cells may also be used.

Any method of introducing the nucleotide sequence into a cell may be employed, including but not limited to, electroporation, calcium phosphate precipitation, microinjection, cationic or anionic liposomes, liposomes in combination with a nuclear localization signal, naturally occurring liposomes (e.g., exosomes), or viral transduction.

For in vivo delivery of an expression vector, viral transduction may be used. A variety of viral vectors known in the art may be adapted by one of skill in the art for use in the present disclosure, for example, vaccinia vectors, adenoviral vectors, lentiviral vectors, poxyviral vectors, adeno-associated viral (AAV) vectors, retroviral vectors, and hybrid viral vectors. In some embodiments, the viral vector used herein is a recombinant AAV (rAAV) vector. AAV vectors are especially suitable for CNS gene delivery because they infect both dividing and non-dividing cells, exist as stable episomal structures for long term expression, and have very low immunogenicity (Hadaczek et al., Mol Ther. (2010) 18:1458-61; Zaiss, et al., Gene Ther. (2008) 15:808-16). Any suitable AAV serotype may be used. For example, the AAV may be AAV1, AAV2, AAV3, AAV3B, AAV4, AAV5, AAV6, AAV7, AAV8, AAV8.2, AAV9, or AAVrh10, or of a pseudotype such as AAV2/8, AAV2/5, AAV2/6, or AAV2/9, or a serotype that is the variant or derivative of one of the AAV serotypes listed herein (i.e., AAV derived from multiple serotypes; for example, the rAAV comprises AAV2 inverted terminal repeats (ITR) in its genome and an AAV8, 5, 6, or 9 capsid). In some embodiments, the expression vector is an AAV viral vector and is introduced to the target human cell by a recombinant AAV virion whose genome comprises the construct, including having the AAV Inverted Terminal Repeat (ITR) sequences on both ends to allow the production of the AAV virion in a production system such as an insect cell/baculovirus production system or a mammalian cell production system. The AAV may be engineered such that its capsid proteins have reduced immunogenicity or enhanced transduction ability in humans or nonhuman primates. In some embodiments, AAV9 is used. Viral vectors described herein may be produced using methods known in the art. Any suitable permissive or packaging cells may be employed to produce the viral particles. For example, mammalian or insect cells may be used as the packaging cell line.

### V. Pharmaceutical Applications

The present ZFP-TFs can be used to treat patients in need of downregulation of alpha-synuclein expression. The patients suffer from, or are at risk of developing, neurodegenerative diseases such as Parkinson's disease, Lewy body dementia, Alzheimer's disease, multiple system atrophy, and any other synucleinopathies. Patients at risk include those who are genetically predisposed, those who have suffered repeated brain injuries such as concussions, and those who have been exposed to environmental neurotoxins. The present disclosure provides a method of treating a neurological disease (e.g., a neurodegenerative disease) in a subject such as a human patient in need thereof, comprising introducing to the nervous system of the subject a therapeutically effective amount (e.g., an amount that allows sufficient repression of SNCA expression) of the ZFP-TF (e.g., an rAAV vector expressing it). The term “treating” encompasses alleviation of symptoms, prevention of onset of symptoms, slowing of disease progression, improvement of quality of life, and increased survival.

The present disclosure provides a pharmaceutical composition comprising a viral vector such as a recombinant AAV (rAAV) whose recombinant genome comprises an expression cassette for the ZFP-TFs. The pharmaceutical composition may further comprise a pharmaceutically acceptable carrier such as water, saline (e.g., phosphate-buffered saline), dextrose, glycerol, sucrose, lactose, gelatin, dextran, albumin, or pectin. In addition, the composition may contain auxiliary substances, such as, wetting or emulsifying agents, pH-buffering agents, stabilizing agents, or other reagents that enhance the effectiveness of the pharmaceutical composition. The pharmaceutical composition may contain delivery vehicles such as liposomes, nanocapsules, microparticles, microspheres, lipid particles, and vesicles.

The cells targeted by the therapeutics of the present disclosure are cells in the brain, including, without limitation, a neuronal cell (e.g., a motor neuron, a sensory neuron, a dopaminergic neuron, a cholinergic neuron, a glutamatergic neuron, a GABAergic neuron, or a serotonergic neuron); a glial cell (e.g., an oligodendrocyte, an astrocyte, a pericyte, a Schwann cell, or a microglial cell); an ependymal cell; or a neuroepithelial cell. The brain regions targeted by the therapeutics may be those most significantly affected in synucleinopathies, such as the striatum, caudate, putamen, substantia nigra, midbrain, olfactory bulb, cerebellum, locus coeruleus, pons, medulla, brainstem, globus pallidus, hippocampus, and cerebral cortex, or other brain regions. These regions can be reached directly through intrastriatal injection, intranigral injection, intracerebral injection, intra-cisterna magna (ICM) injection, or more generally through intraparenchymal injection, intracerebroventricular (ICV) injection, intrathecal injection, or intravenous injection. Other routes of administration include, without limitation, intraventricular, intranasal, or intraocular administration. In some embodiments, the viral vector spreads throughout the CNS tissue following direct administration into the cerebrospinal fluid (CSF), e.g., via intrathecal and/or intracerebroventricular injection, or intracisterna-magna injection. In other embodiments, the viral vectors cross the blood-brain barrier and achieve wide-spread distribution throughout the CNS tissue of a subject following intravenous administration. In other embodiments, the viral vectors are delivered directly to the target regions via intraparenchymal injections. In some cases, the viral vectors may undergo retrograde or anterograde transport to other brain regions following intraparenchymal delivery. In some aspects, the viral vectors have distinct CNS tissue targeting capabilities (e.g., CNS tissue tropisms), which achieve stable and nontoxic gene transfer at high efficiencies.

By way of example, the pharmaceutical composition may be provided to the patient through intraventricular administration, e.g., into a ventricular region of the forebrain of the patient such as the right lateral ventricle, the left lateral ventricle, the third ventricle, or the fourth ventricle. The pharmaceutical composition may be provided to the patient through intracerebral administration, e.g., injection of the composition into or near the striatum, caudate, putamen, substantia nigra, midbrain, olfactory bulb, cerebrum, cerebellum, locus coeruleus, pons, medulla, brainstem, globus pallidus, hippocampus, cerebral cortex, intracranial cavity, meninges, dura mater, arachnoid mater, or pia mater of the brain. Intracerebral administration may include, in some cases, administration of an agent into the cerebrospinal fluid (CSF) of the subarachnoid space surrounding the brain.

In some cases, intracerebral administration involves injection using stereotaxic procedures. Stereotaxic procedures are well known in the art and typically involve the use of a computer and a 3-dimensional scanning device that are used together to guide injection to a particular intracerebral region, e.g., a ventricular region. Micro-injection pumps (e.g., from World Precision Instruments) may also be used. In some cases, a microinjection pump is used to deliver a composition comprising a viral vector. In some cases, the infusion rate of the composition is in a range of 0.1 μl/min to 100 μl/min. As will be appreciated by the skilled artisan, infusion rates will depend on a variety of factors, including, for example, species of the subject, age of the subject, weight/size of the subject, serotype of the AAV, dosage required, and intracerebral region targeted. Thus, other infusion rates may be deemed by a skilled artisan to be appropriate in certain circumstances.

Delivery of rAAVs to a subject may be accomplished, for example, by intravenous administration. In certain instances, it may be desirable to deliver the rAAVs locally to the brain tissue, the spinal cord, cerebrospinal fluid (CSF), neuronal cells, glial cells, meninges, astrocytes, oligodendrocytes, microglia, interstitial spaces, and the like. In some cases, recombinant AAVs may be delivered directly to the CNS by injection into the ventricular region, as well as to the striatum, caudate, putamen, substantia nigra, midbrain, olfactory bulb, cerebellum, locus coeruleus, pons, medulla, brainstem, globus pallidus, hippocampus, cerebral cortex, or other brain region. AAVs may be delivered with a needle, catheter or related device, using neurosurgical techniques known in the art, such as by stereotactic injection (see, e.g., Stein et al., J Vir. (1999) 73:3424-9; Davidson et al., PNAS. (2000) 97:3428-32; Davidson et al., Nat Genet. (1993) 3:219-223; and Alisky and Davidson, Hum. Gene Ther. (2000) 11:2315-29).

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of neurology, medicine, medicinal and pharmaceutical chemistry, and cell biology described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

In order that this invention may be better understood, the following examples are set forth. These examples are for purposes of illustration only and are not to be construed as limiting the scope of the invention in any manner.

### EXAMPLES

### Example 1: Screening of ZFP-TFs

In order to identify ZFP-TFs that repress the expression of alpha-synuclein, we designed and screened a library of 416 ZFP-TFs predicted to bind 15 or 18 bp sequences in the region of the human SNCA gene spanning from 500 bps upstream of TSS 1 to 500 bps downstream of TSS 1 or 500 bp upstream of TSS 2a to 500 bp downstream of TSS 2b (FIG. 2B). The target regions of the ZFP-TFs are denoted by arrowheads in FIG. 2B, with the direction of the arrowhead indicating the strand of DNA the ZFP-TF binds to (5′ to 3′). FIG. 4 shows similar data to those displayed in FIG. 2B for ZFP-TFs with 1, 2, or 3 phosphate contact changes targeting TSSs 2a and 2b of the SNCA gene. In this study, a KRAB domain sequence (SEQ ID NO: 12) was used as the transcription repressor and fused to the C-terminus of the ZFP domain. The sequences of 47 representative ZFP-TFs are shown in Table 2 below. Templates for in vitro transcription were generated from pVAX-ZFP or pVAX-GFP plasmids using PCR (forward primer GCAGAGCTCTCTGGCTAACTAGAG (SEQ ID NO: 13); reverse primer T(180)CTGGCAACTAGAAGGCACAG (SEQ ID NO: 14). Messenger RNA was synthesized using a mMESSAGE mMACHINE T7 ULTRA Transcription Kit (Thermo Fisher Scientific) as per the manufacturer's instructions and purified using RNeasy96 columns (Qiagen). RNA encoding each ZFP-TF was then aliquoted into 96-well plates in a 6-dose dilution.

Recombinant rAAV vectors carrying the ZFP-TF coding sequences were generated in HEK293 cells according to well-known methods. Three days after the cells were transfected with plasmids encoding AAV helper genes and the rAAV genome, the cells were harvested. The cells were then lysed by three rounds of freeze/thaw and the cell debris was removed by centrifugation. The rAAV virions were precipitated using polyethylene glycol. After resuspension, the virions were purified by ultracentrifugation overnight on a cesium chloride gradient. The virions were formulated by dialysis and then filter-sterilized. The AAVs were aliquoted and stored at −80° C. until use. The AAVs were not re-frozen after thawing.

The screening was performed in the SK-N-MC human neuroepithelial cell line. SK-N-MC cells express human alpha-synuclein at high levels and are thus appropriate for testing of ZFP-TFs that reduce alpha-synuclein expression. The SK-N-MC cells were cultured in tissue culture flasks until confluency. The cells were plated on 96-well plates at 150,000 cells per well and were resuspended in Amaxa® SF solution. The cells were then mixed with ZFP-TF RNA (6 doses: 3, 10, 30, 100, 300, and 1000 ng) and transferred to Amaxa® shuttle plate wells. The cells were transfected using the Amaxa® Nucleofector® device (Lonza; program CM-137). Eagle's MEM cell media was added to each well of the plate. The cells were transferred to a 96-well tissue culture plate and incubated at 37° C. for 24 hours.

The ZFP-TFs were also tested in human iPSC-derived GABAergic neurons (Cellular Dynamics International). The cells were plated onto poly-L-ornithine- and laminin-coated 96-well plates at a density of 40,000 cells per well and then maintained according to the manufacturer's instructions. The cells were transfected with AAV6 expressing the desired ZFP-TF at 6 different MOIs (1E3, 3E3, 1E4, 3E4, 1E5, and 3E5) 48 hours after plating. The transduced cells were maintained for up to 32 days (50-75% media changes performed every 3-5 days). The cells were harvested 28-30 days after AAV transfection.

Harvested cells were lysed and reverse transcription was performed using the C2CT kit following the manufacturer's instructions. TaqMan quantitative polymerase chain reaction (qPCR) was used to measure the expression levels of SNCA. SNCA expression levels were normalized to the geometric mean of the expression levels of the housekeeping genes EIF4A2, ATP5B and GAPDH. A mock transfection and transfection with a ZFP-TF known not to target SNCA were used as negative controls.

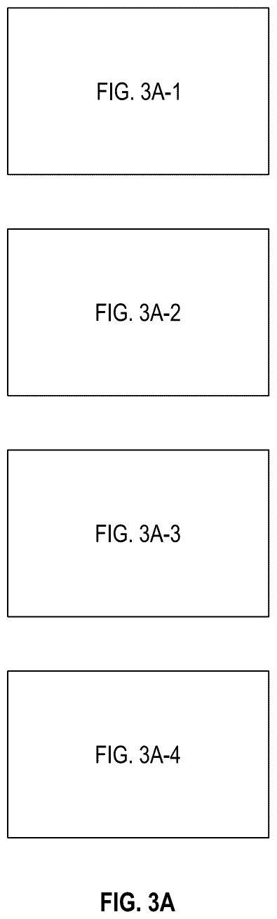

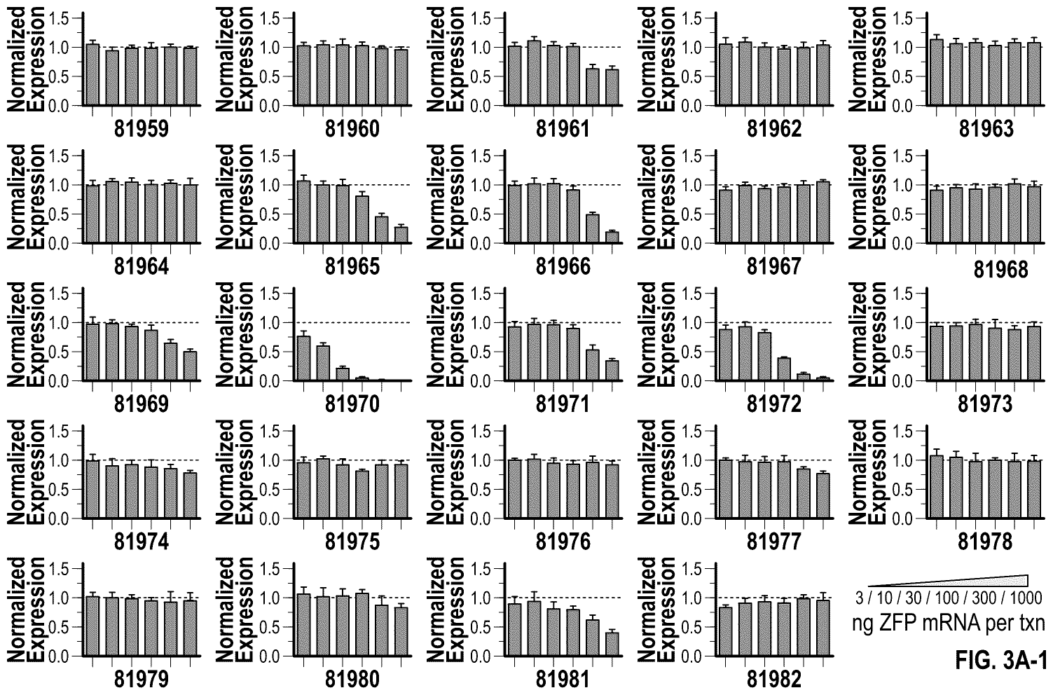

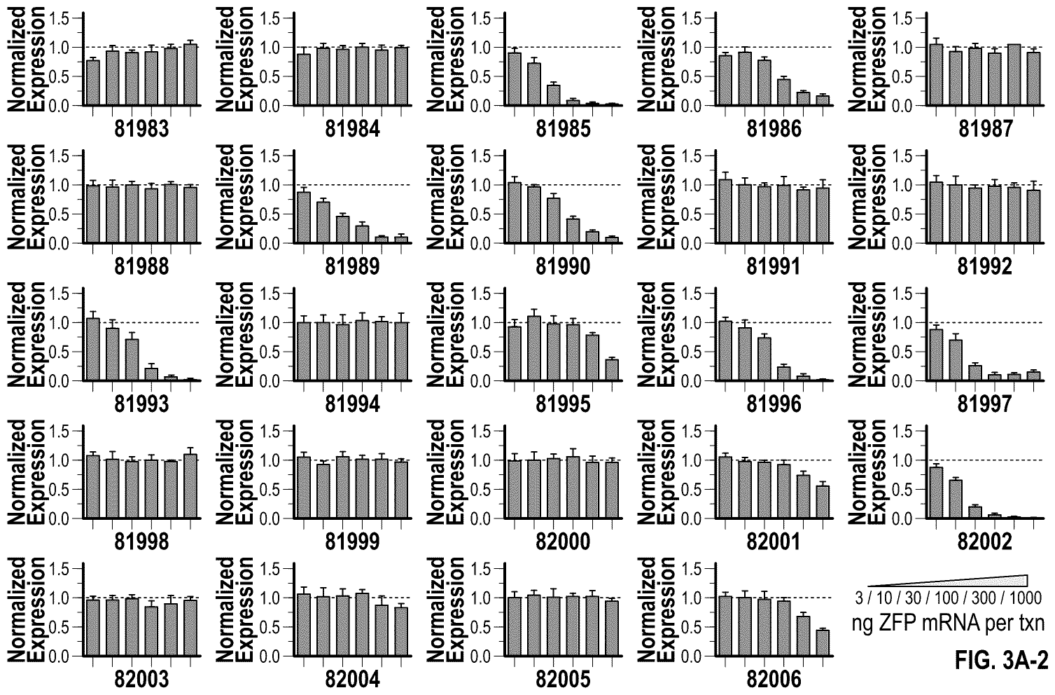

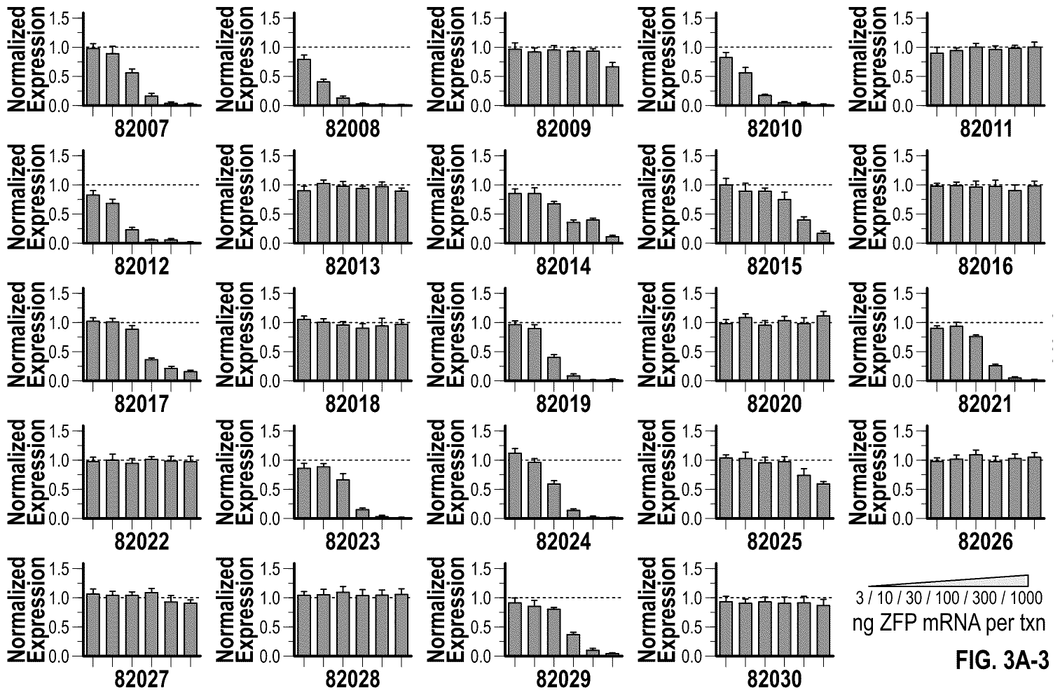

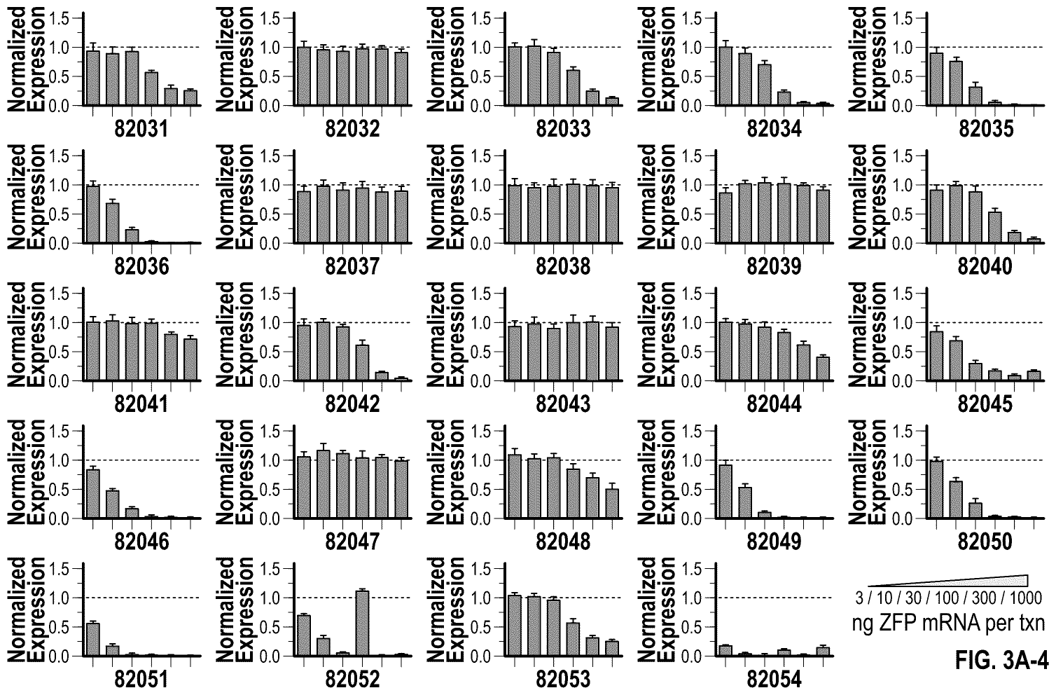

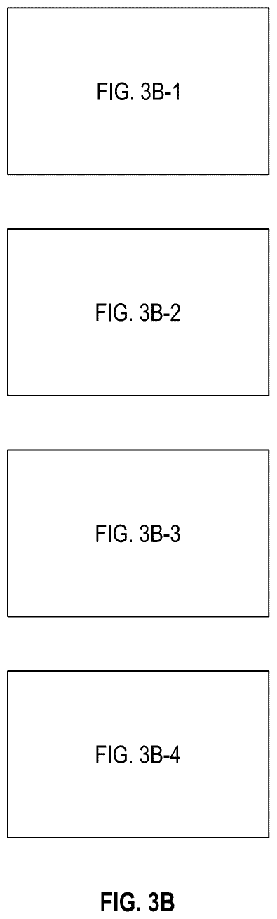

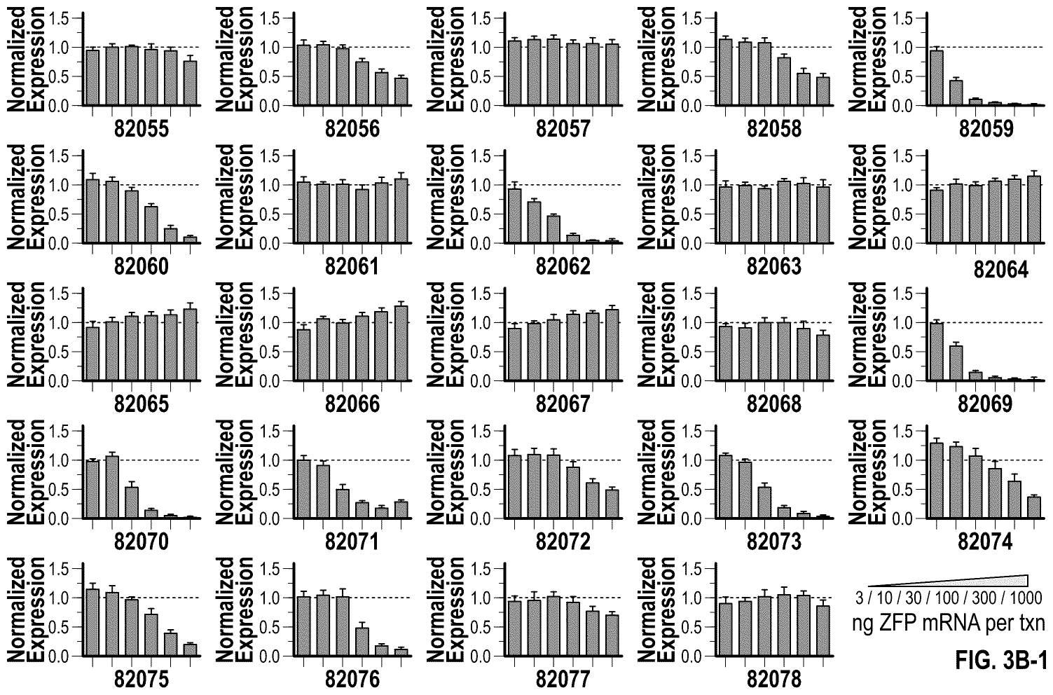

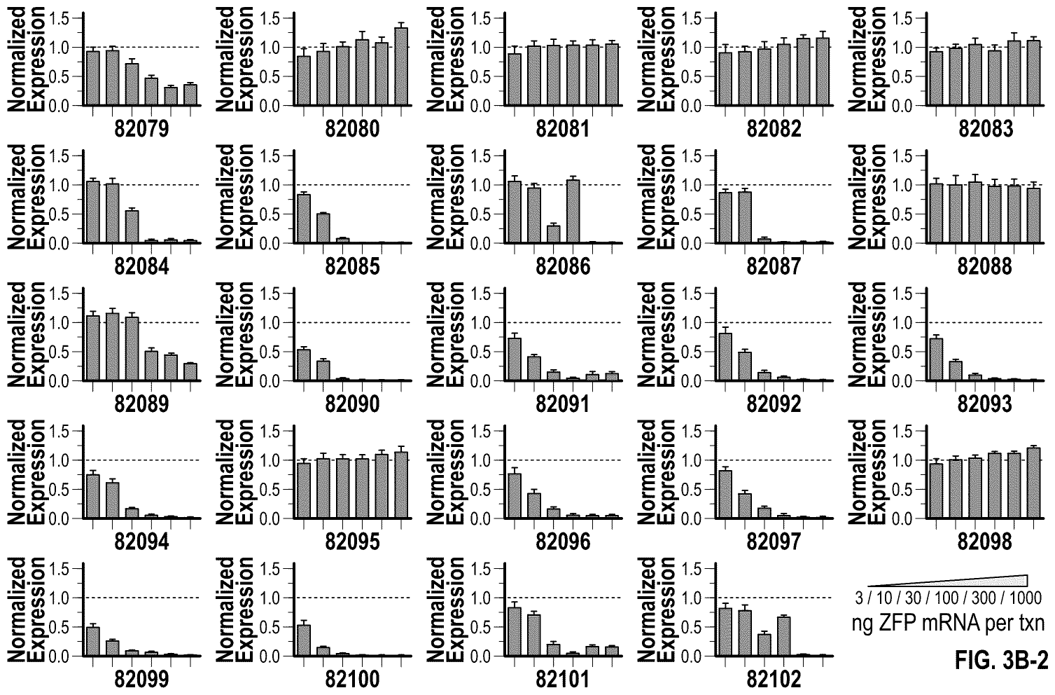

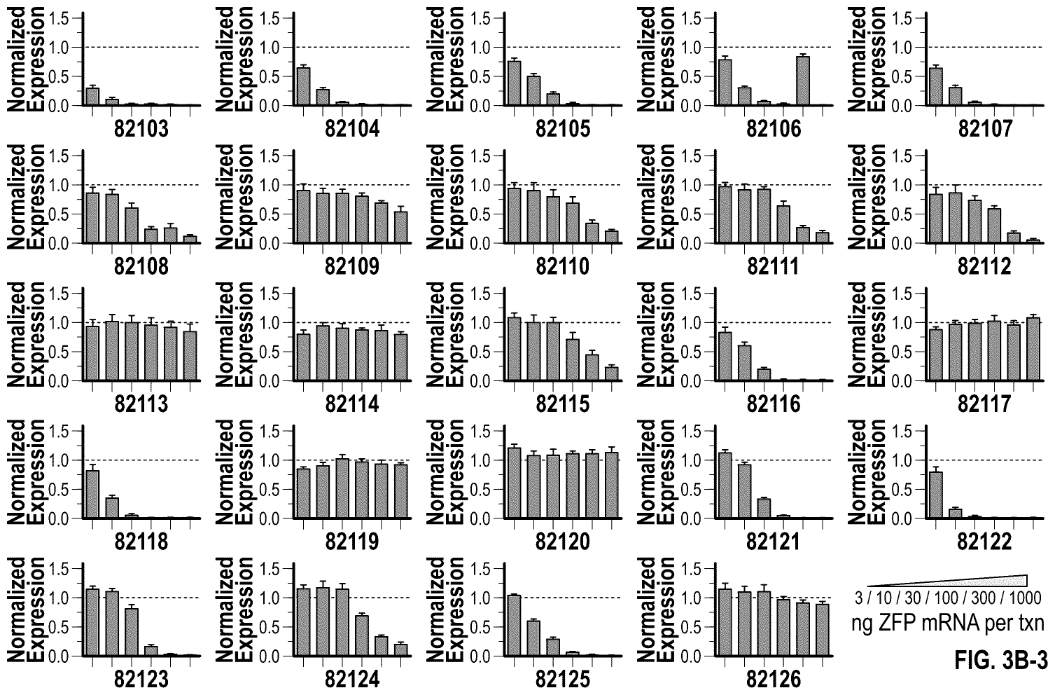

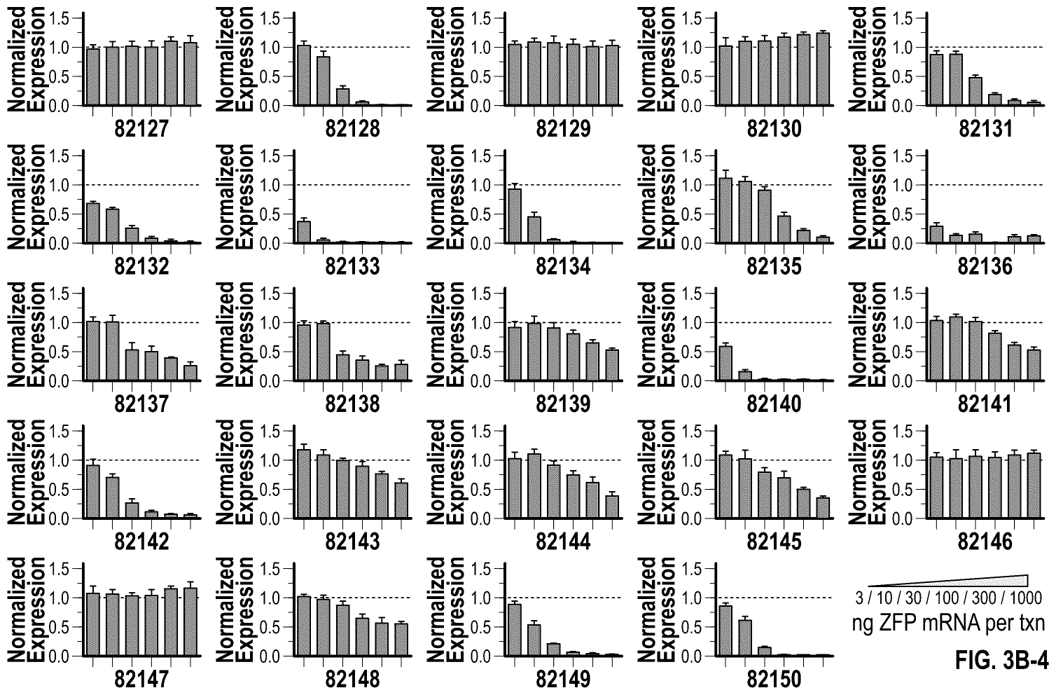

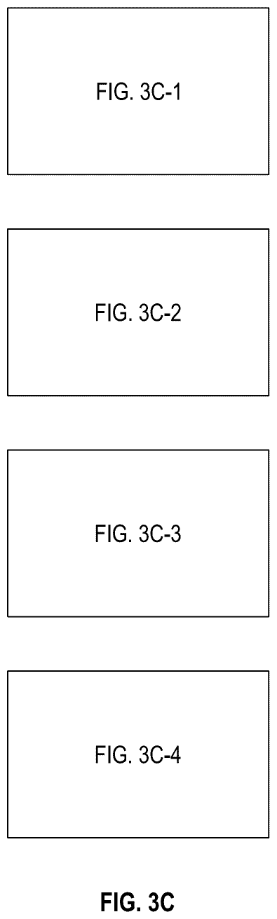

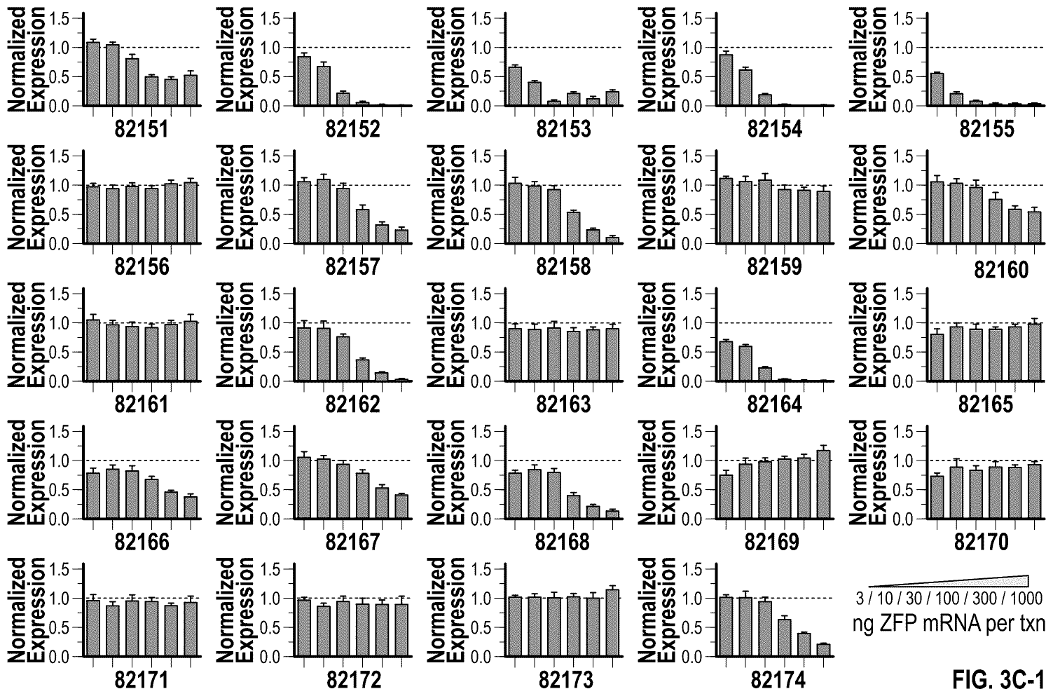

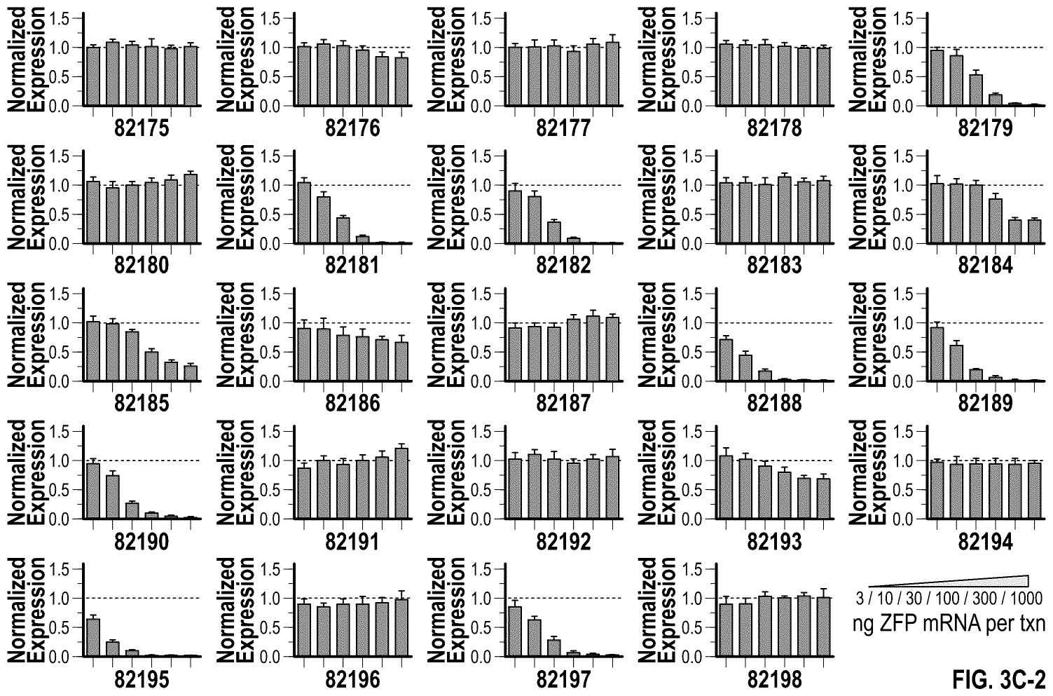

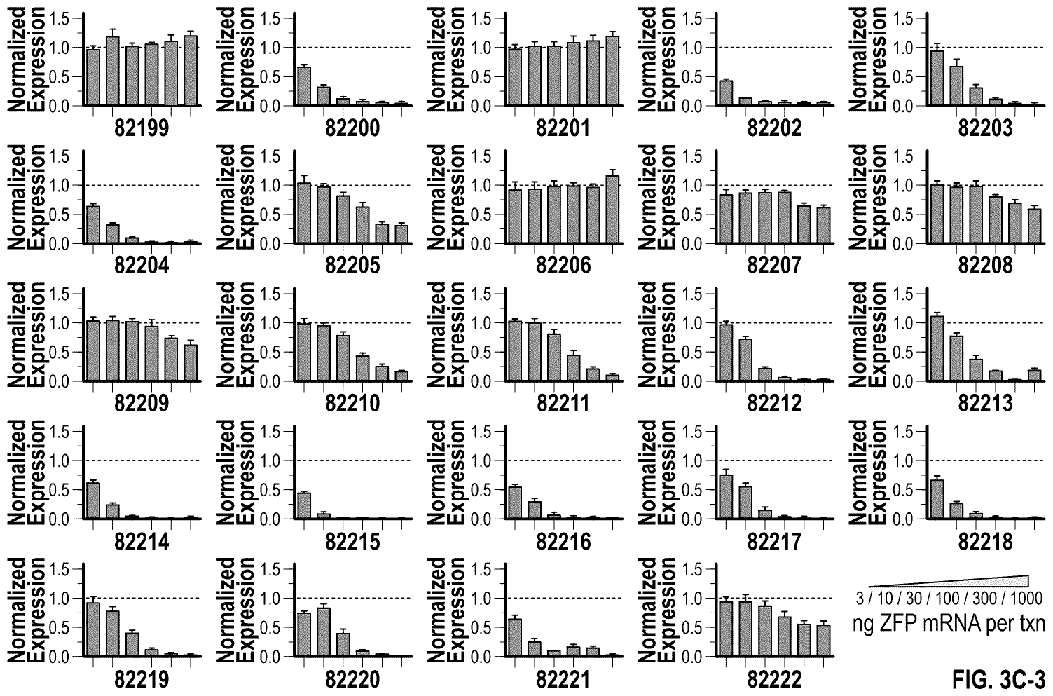

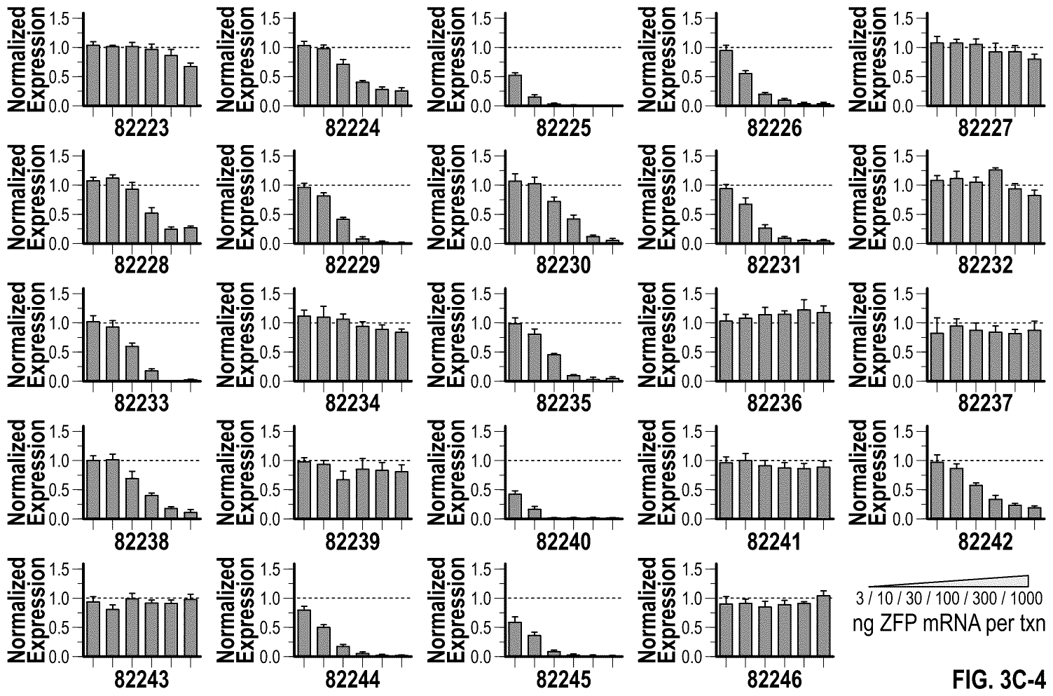

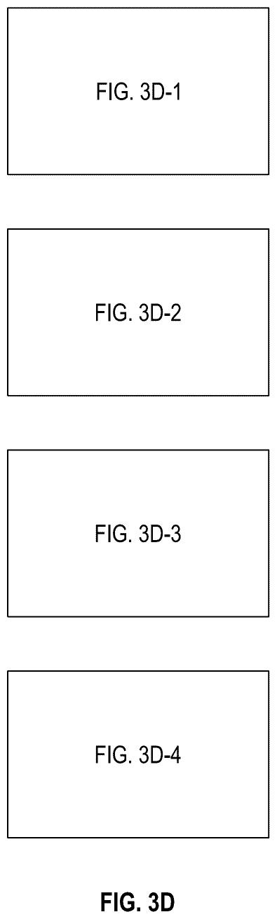

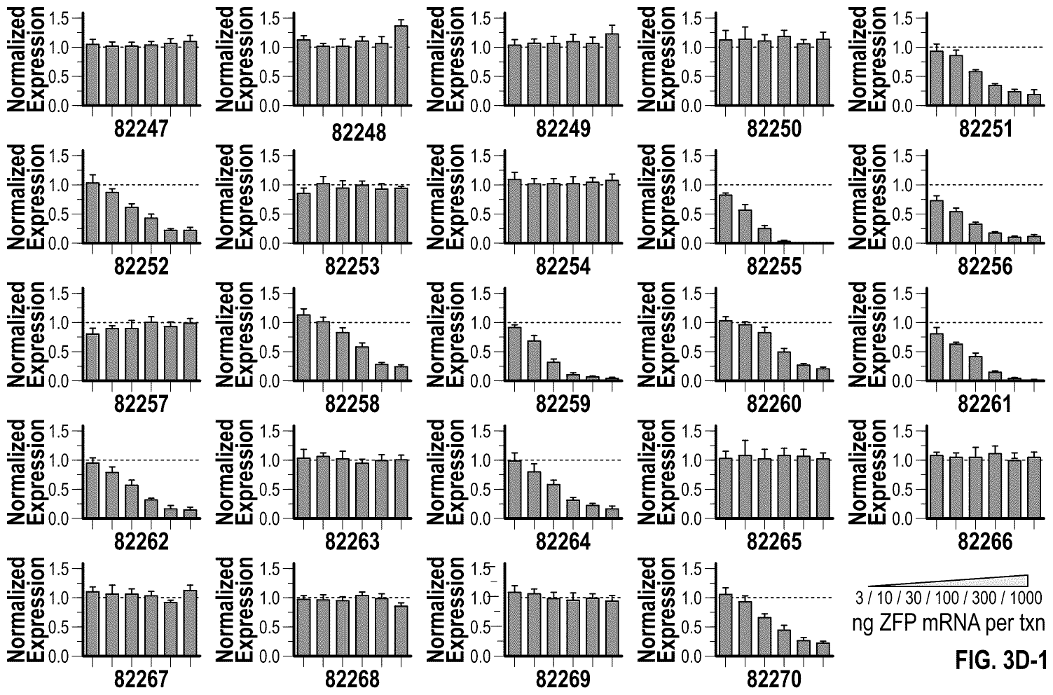

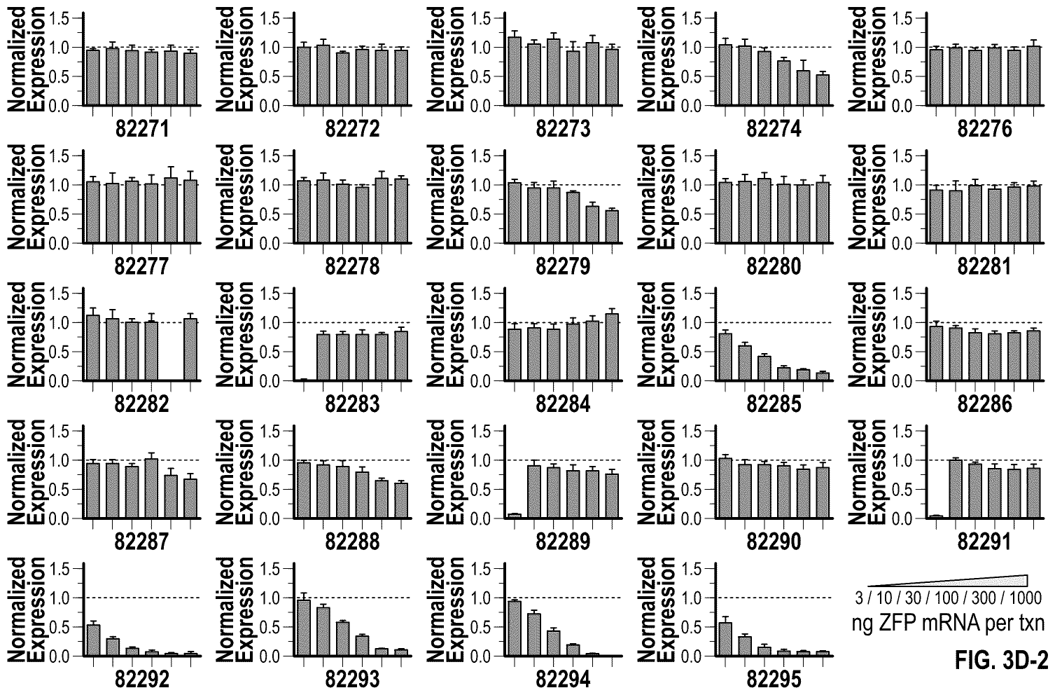

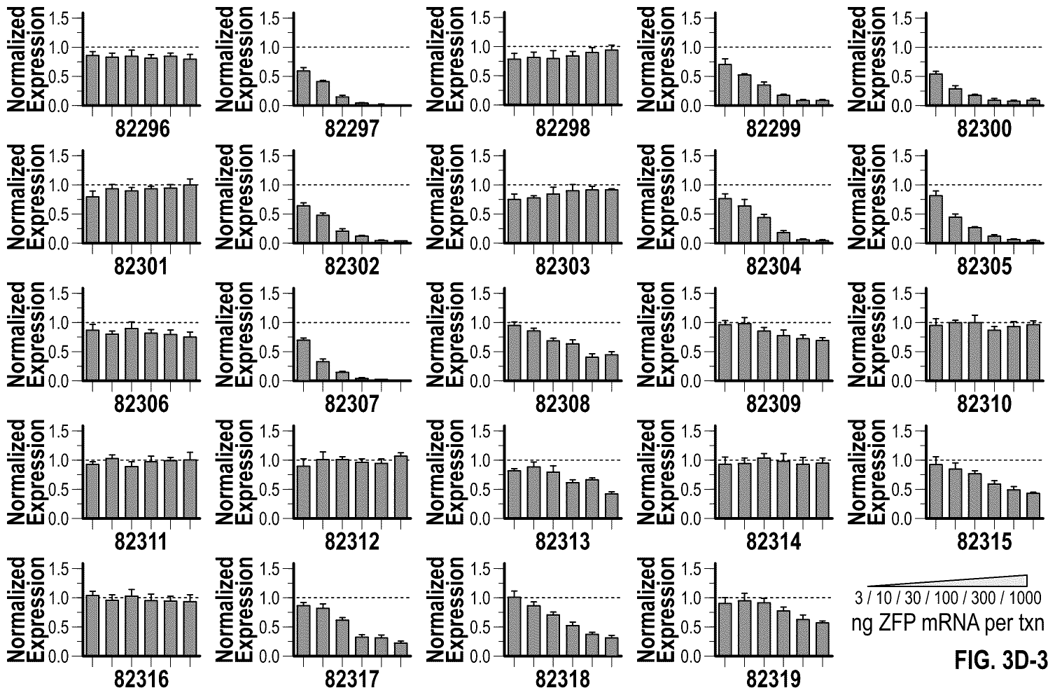

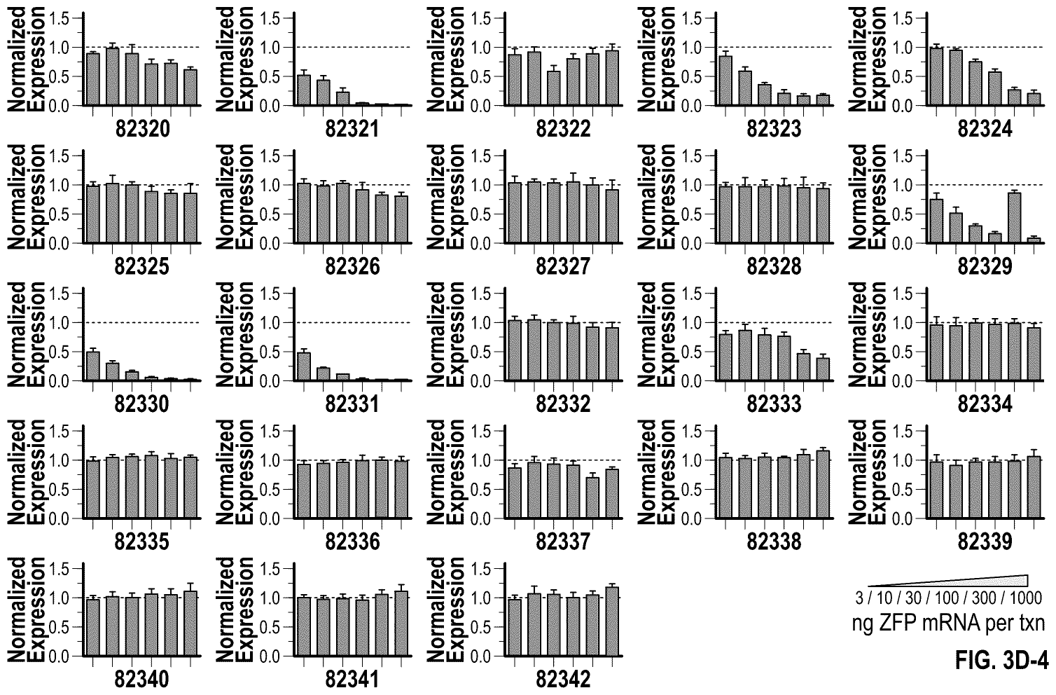

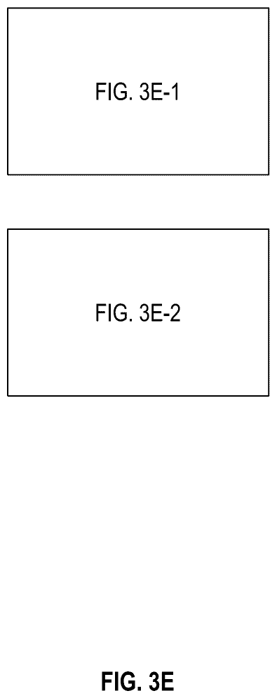

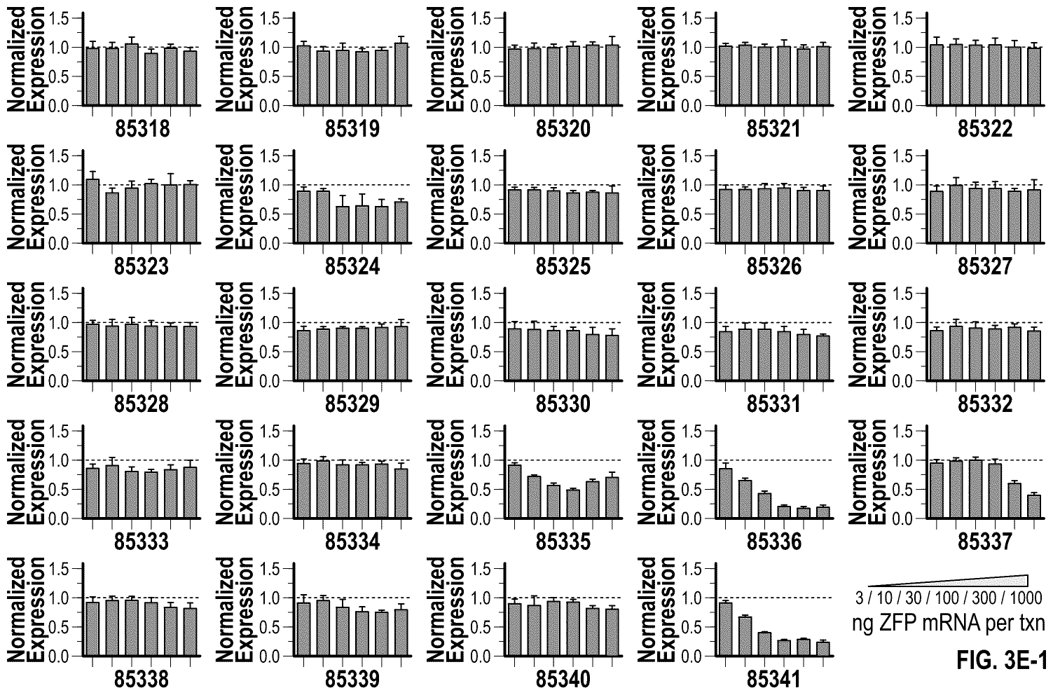

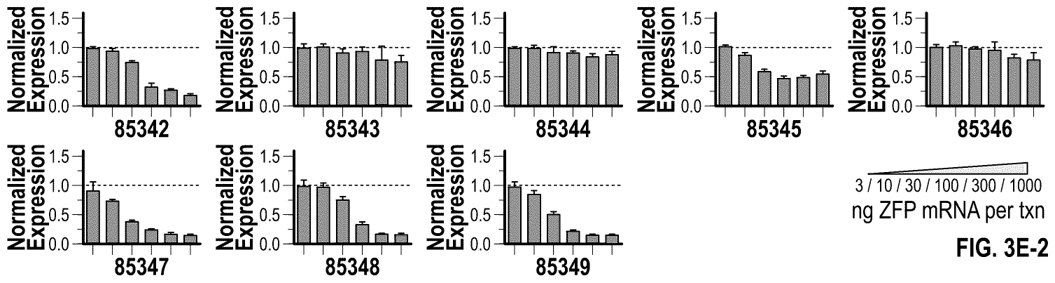

Dose-dependent repression of alpha-synuclein was demonstrated with many of the ZFP-TFs tested. The maximum repression achieved was more than 99%, but we also identified ZFP-TFs that repressed alpha-synuclein to a lesser degree (e.g., about 90%, about 75%, or about 40% at the highest dose). FIGS. 3A-E and 5 show the screening data.

### Example 2: Off-Target Activity of Alpha-Synuclein ZFP-TFs

To evaluate the off-target impact of the alpha-synuclein ZFP-TFs on global gene expression, we performed microarray experiments on total RNA isolated from human iPSC-derived neurons and primary mouse cortical neurons treated with AAVs encoding the representative alpha-synuclein ZFP-TFs.

Human iPSC-derived neurons were treated as described in Example 1. For microarray analysis, the cells were plated onto poly-L-omithine- and laminin-coated 24-well plates at a density of 260,000 cells per well, transfected with 1E5 VGs/cell 48 hours after plating, and harvested 19 days after viral transfection. RNA isolated from the harvested cells was used for microarray analysis.

Primary mouse cortical neurons were purchased from Gibco. Cells were plated onto poly-D-lysine-coated 24-well plates at 200,000 cells/well and maintained according to the manufacturer's specifications using Gibco Neurobasal Medium containing GlutaMAX™ I supplement, B27 supplement, and penicillin/streptomycin. Forty-eight hours after plating (at DIV2), the cells were infected with AAV6 at an MOI of 3E3 VGs/cell and harvested 7 days later (at DIV9; 50% media exchanges performed every 3-4 days). This was followed by RNA isolation and microarray analysis.

Off-target analysis was performed using the GeneTitan™ platform (Clariom S kit) according to the manufacturer's instructions. The assay results were analyzed using TAC software. Differentially regulated genes with FDR-corrected p-values<0.05 that were regulated by >2-fold were called out in the analysis. A ZFP-TF known to have minimal off-targets and a mock transfection were used as negative controls.

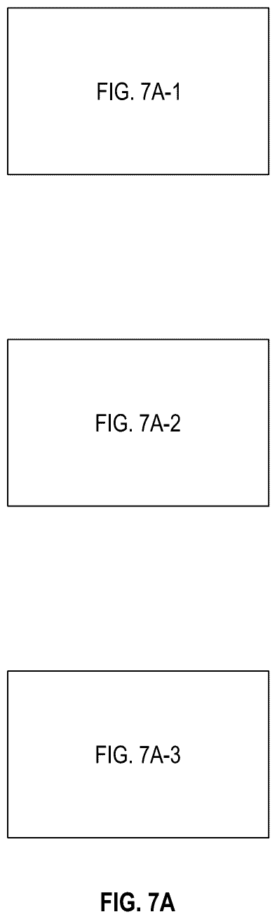

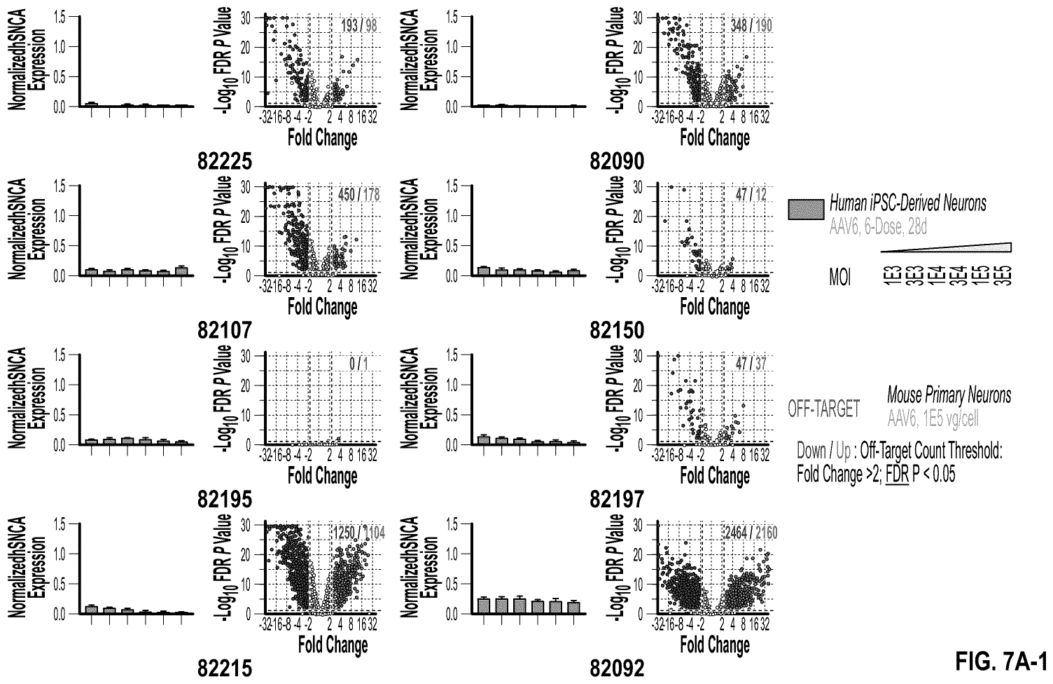

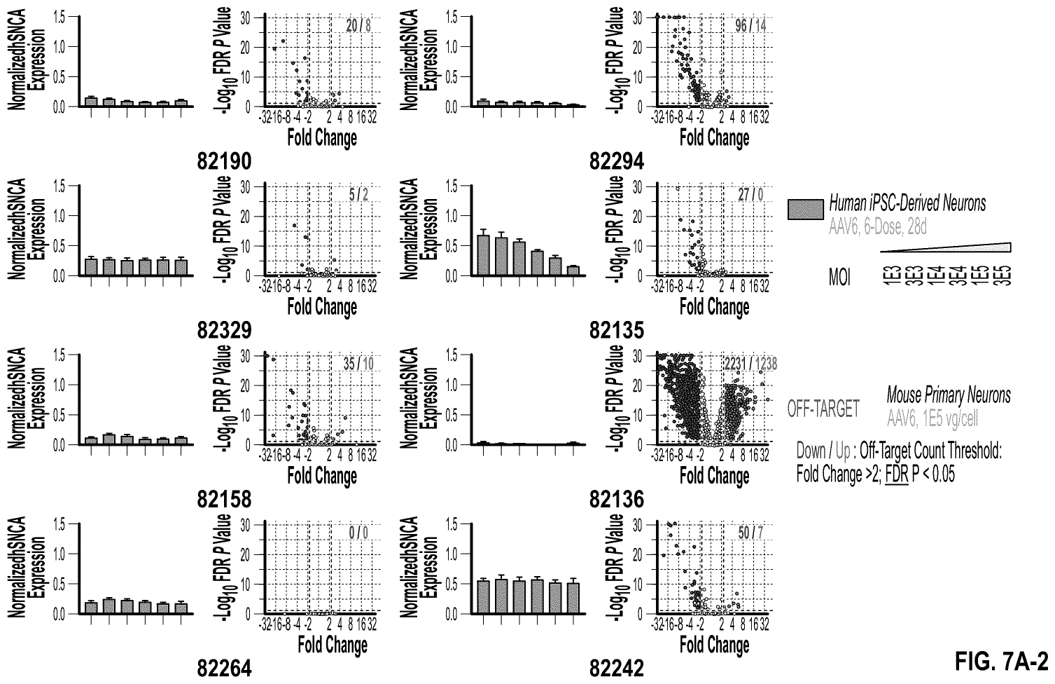

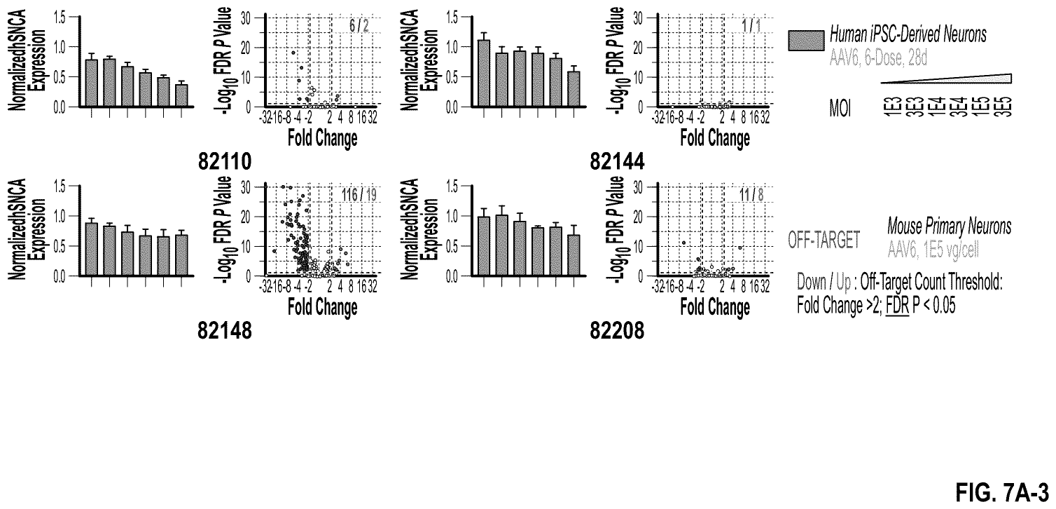

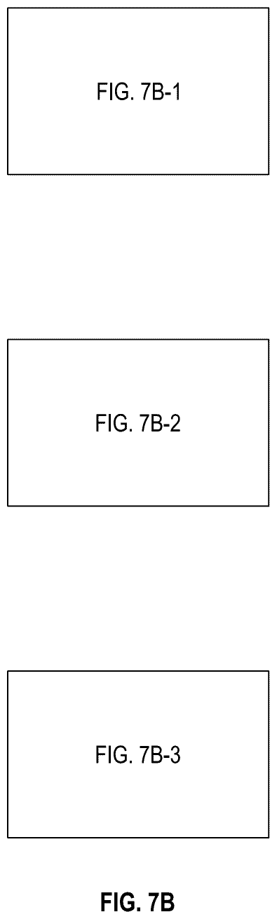

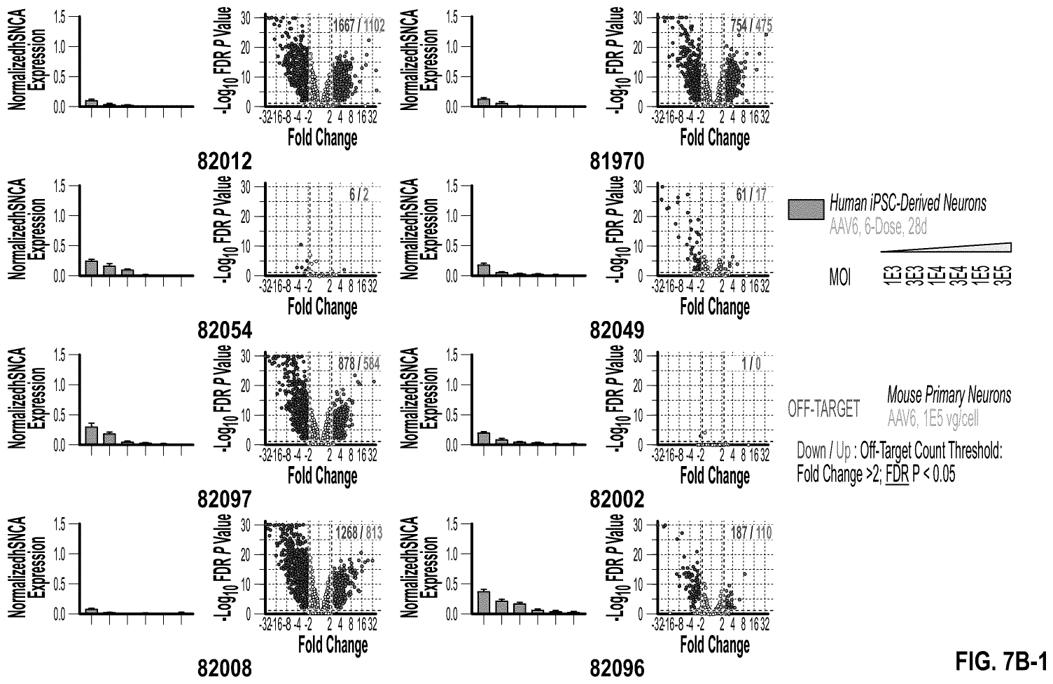

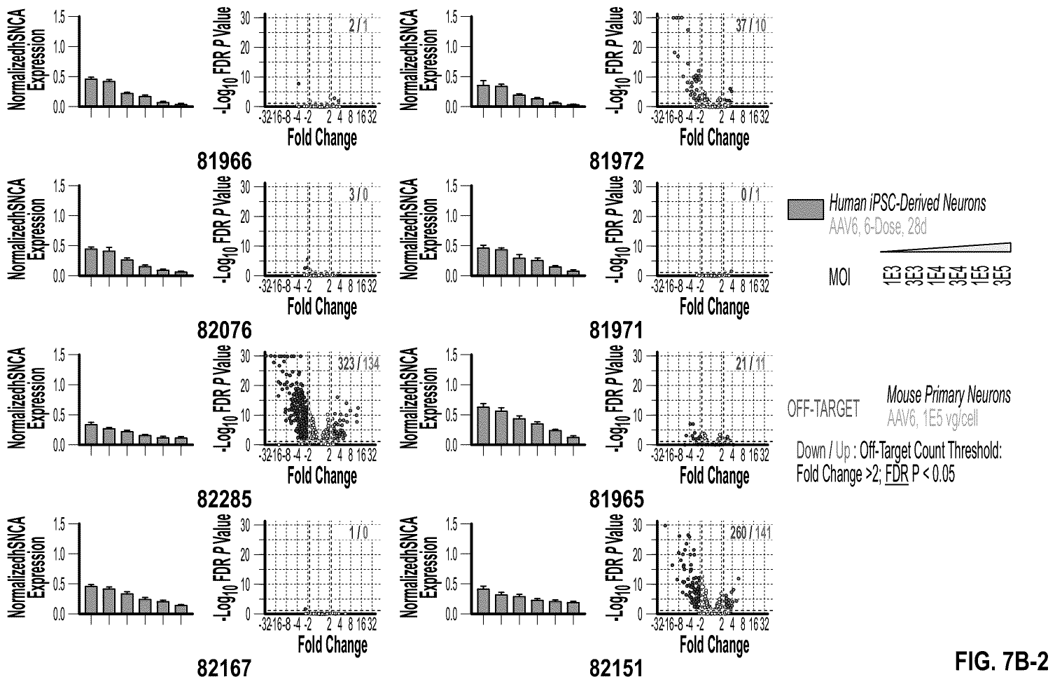

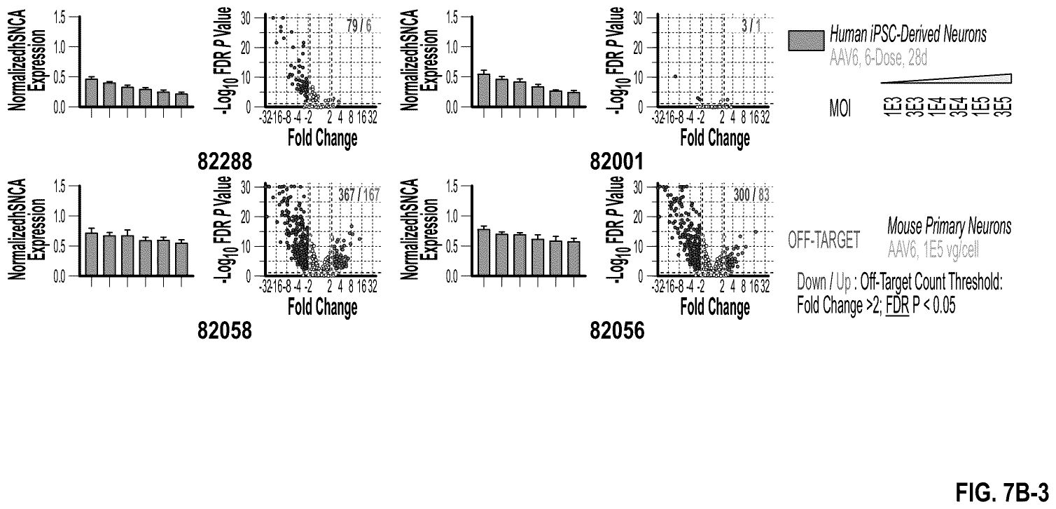

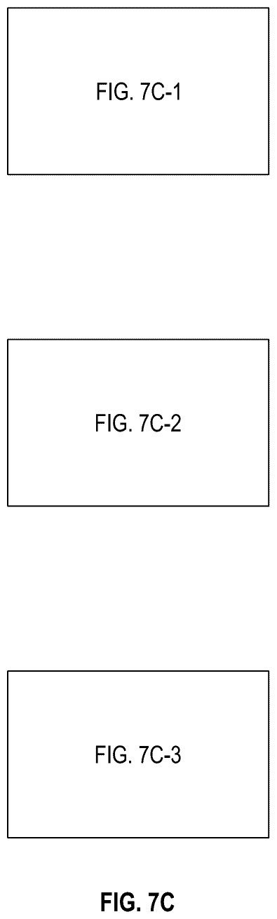

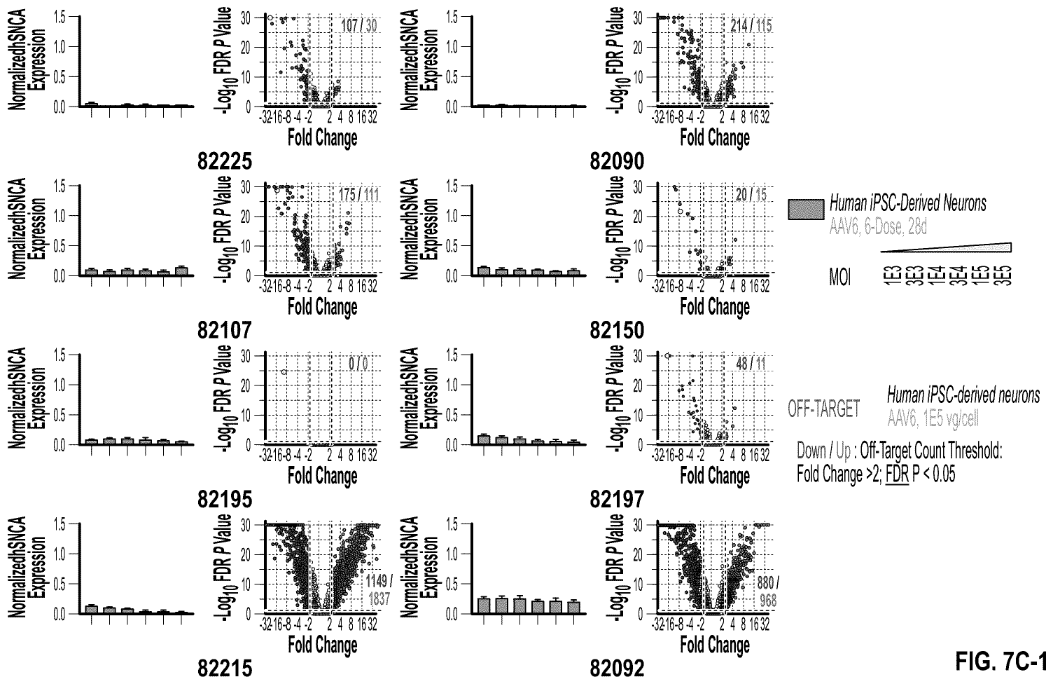

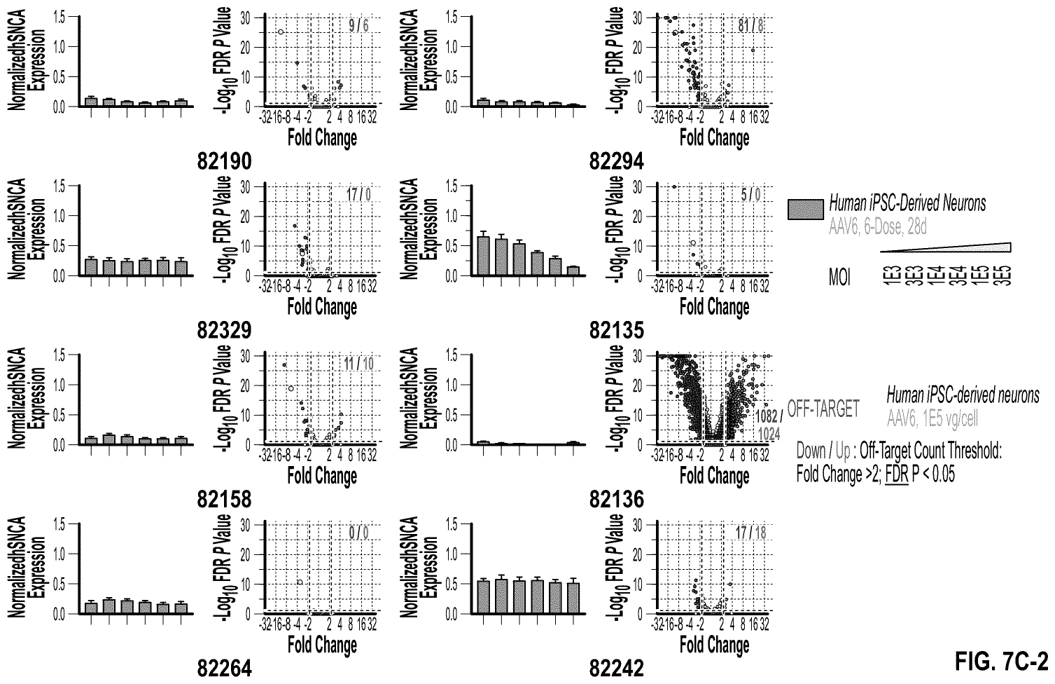

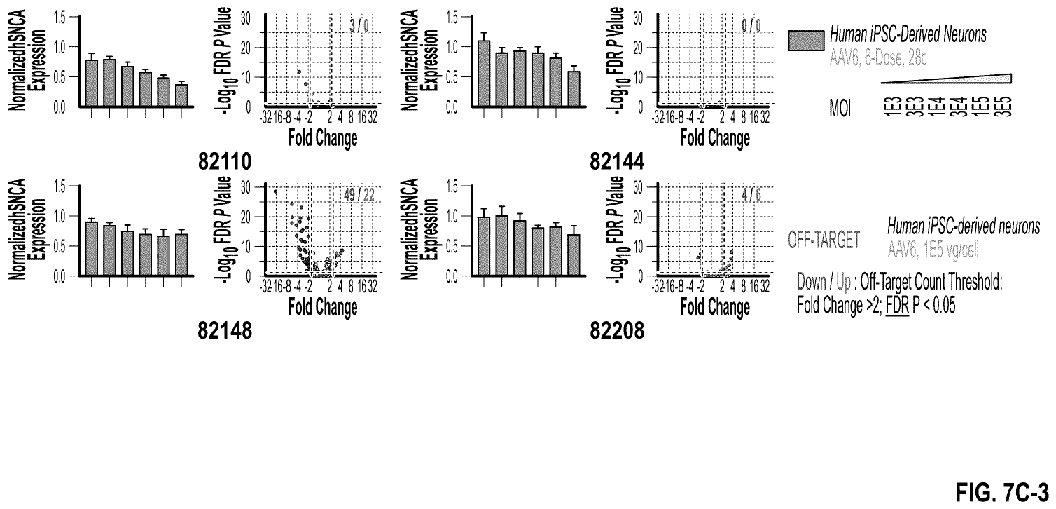

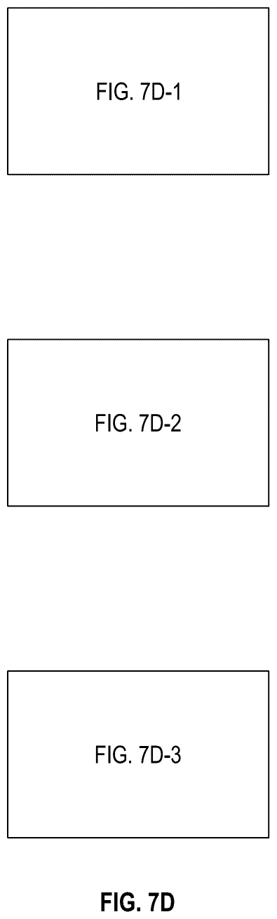

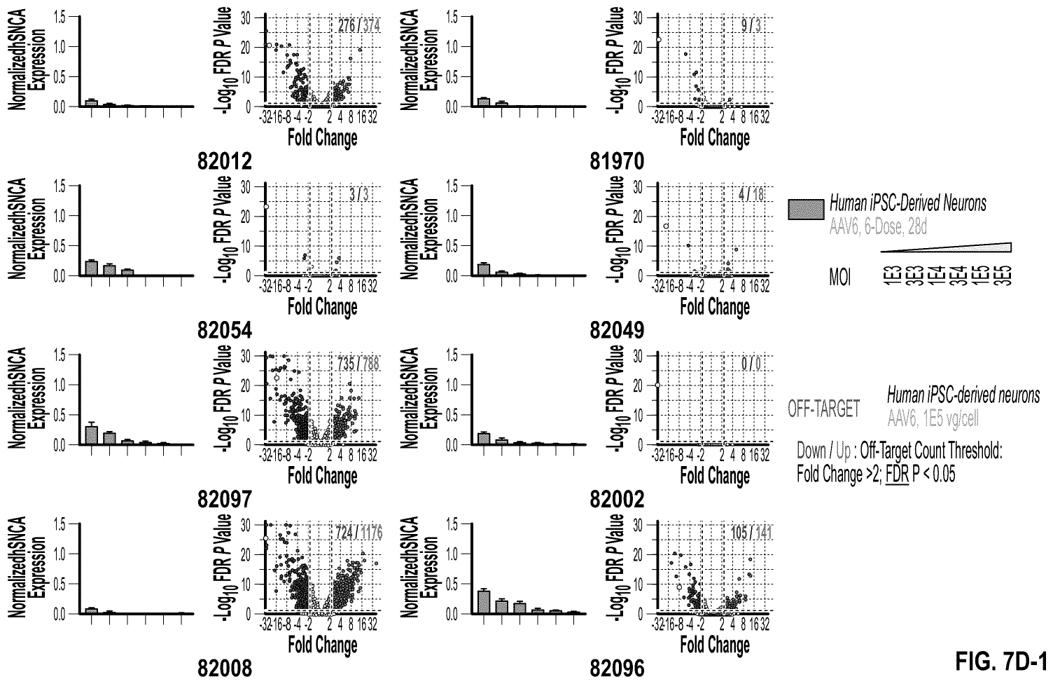

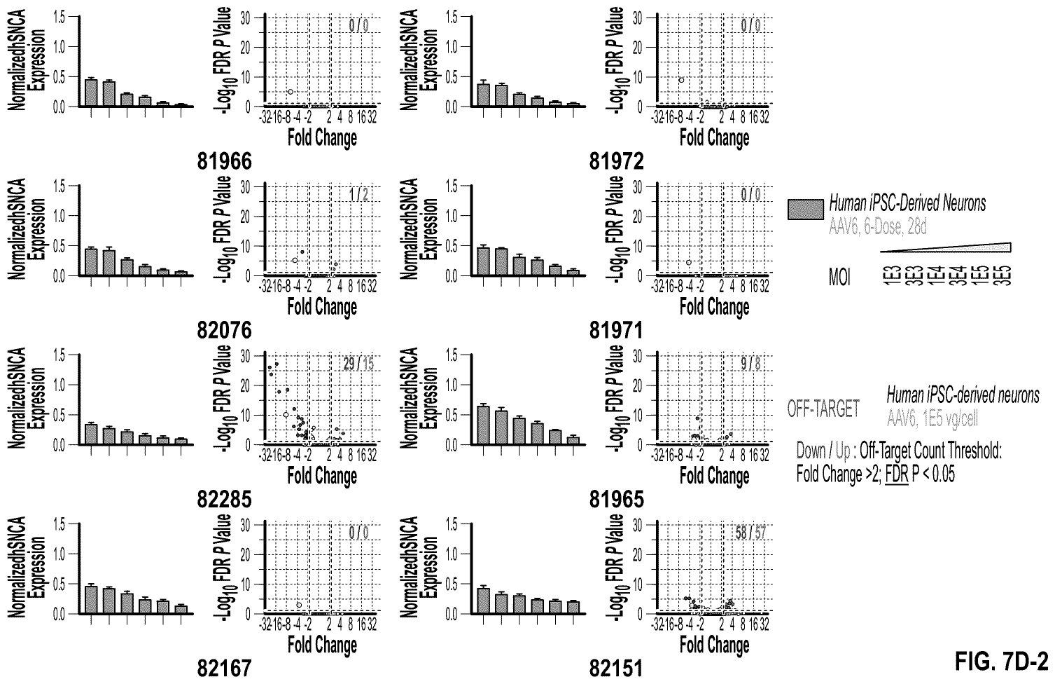

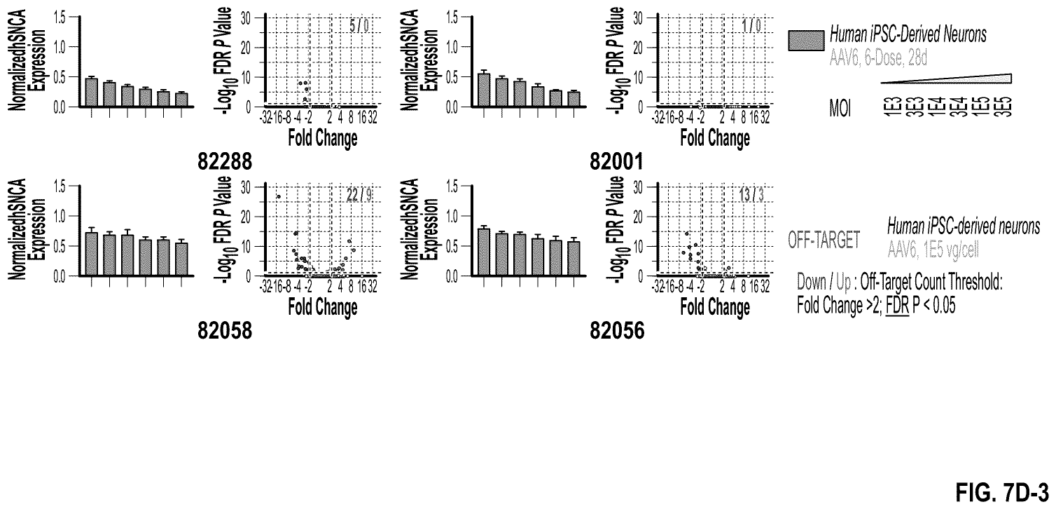

FIGS. 7A-D show the microarray results of 40 representative alpha-synuclein ZFP-TFs in human iPSC-derived neurons and primary mouse cortical neurons. There was a range of off-target activity, with some ZFP-TFs displaying very low to no detectable off-target activity.

### Example 3: In Vivo Repression of Alpha-Synuclein mRNA Expression

AAV9 constructs expressing two representative ZFP-TFs (82195 and 82264) with minimal to no detectable off-target activity in both human and mouse neurons and different maximal repression activity in human iPSC-derived neurons (82195, −95%; 82264, −80%) were used to demonstrate in vivo repression of human SNCA in the PAC synuclein mouse model (Kuo et al., Hum Mol Genet. (2010) 19(9):1633-50). This mouse model expresses the full human SNCA sequence along with its upstream regulatory sequence on a mouse alpha-synuclein-null background. AAV9 vectors expressing each ZFP-TF and vehicle were bilaterally administered to the striatum at two sites in 8-week-old female PAC synuclein mice (n=3 per group) at a rate of 0.5 μL/minute (2 sites per hemisphere: 5 μL to anterior striatum and 4 μl to the posterior striatum, a total of 9 μL/hemisphere and 18 μL/animal). Following infusion, the needle was left in place for 5 minutes to allow the test article to diffuse. The needle was then slowly retracted over 1-2 minutes. The stereotaxic coordinates for the injections were as follows (rostral striatum −AP: +1.4 mm, ML: +/−1.7 mm, DV: −3.0 mm; caudal striatum: AP: +0.2 mm, ML: +/−2.3 mm, DV: −2.7 mm). The mice were euthanized after 3 weeks and their brains were collected for molecular analyses. At the time of euthanasia, the animals were transcardially perfused with 0.9% saline, and the brain was removed and hemisected. The left hemisphere was further dissected into 12 different regions (olfactory bulb; rostral, medial, and caudal cortex; rostral, medial, and caudal striatum; hippocampus; thalamus; ventral midbrain; medulla; and cerebellum). The dissected tissues were placed in RNALater to preserve RNA integrity. After 24 h, RNAlater was removed and the tissues were flash-frozen in liquid nitrogen and maintained on dry ice until storage at −80° C.

Brain tissues were transferred to 1.5 mL Eppendorf tubes containing 0.6 mL TRI reagent (Thermo Fisher) and two 3.2 mm steel beads (BioSpec Products) on ice. The samples were lysed using a Qiagen TissueLyser at 4° C. using the following parameters: 5 cycles, 90 s duration, and 25.1 frequency. After a brief spin, 70 μL of 1-bromo-3-chloropropane was added to each sample at room temperature. The samples were vortexed for 10 s, centrifuged at 12,000×g for 10 min at 4° C., and 120 μL of the aqueous phase from each sample was transferred to wells of a 96-well plate.

Sixty microliters of isopropyl alcohol and 12 μL of MagMax magnetic beads (Thermo Scientific) were added to each well containing the aqueous phase of the tissue lysate. A Kingfisher 96 robot (Thermo Scientific) and the MagMax kit (Thermo Fisher) were used to isolate ribonucleic acid (RNA) from the tissue lysate following the manufacturer's protocol. One hundred microliters of the eluted RNA were separated from the magnetic beads using a magnetic stand. RNA yield and quality were evaluated using a Nanodrop 8000 instrument (Thermo Scientific).

Complementary deoxyribonucleic acid (cDNA) was prepared using the High-Capacity cDNA Reverse Transcription Kit (Applied Biosystems), with 10 μL of RNA and 10 μL of RT Master Mix (10×RT buffer, 10× random primer, 25×dNTP mix, Multiscribe enzyme, and RNAse-free water) by default. If needed, the RNA and RT Master Mix volumes were adjusted to ensure that the input was in the 100 to 1,000 ng range. Reverse transcription was performed on a C1000 Touch Biorad thermal cycler using the following program: 25° C. for 10 min, 37° C. for 120 min, 85° C. for 5 min, and hold at 4° C.

cDNA was subjected to RT-qPCR using Biorad CFX384 thermal cyclers. cDNA was diluted 10 fold in nuclease-free water, and 4 μL of diluted cDNA were added to each 10 μL PCR reaction. Each sample was assayed in technical quadruplicate. 2× Fast Multiplex PCR (Qiagen) master mix was used for triplex assays, and SsoAdvanced Universal Probes Supermix (Biorad) was used for other assays.

The following cycling conditions were used: Qiagen Fast Multiplex master mix→95° C. for 5 min, 95° C. for 45 s, 60° C. for 45 s, plate read, 40 cycles; Biorad SsoAdvanced master mix→95° C. for 90 s, 95° C. for 12 s, 60° C. for 40 s, plate read, 42 cycles.

Spiking of GFP RNA before vs. after the RNA isolation step was used to assess % RNA recovery. A 4-sample 5-fold dilution series of pooled RNA from the animals in the study was used as a standard curve. Sample data were normalized to the geometric mean of the three housekeeping genes ATP5B, EIF4A2, and GAPDH.

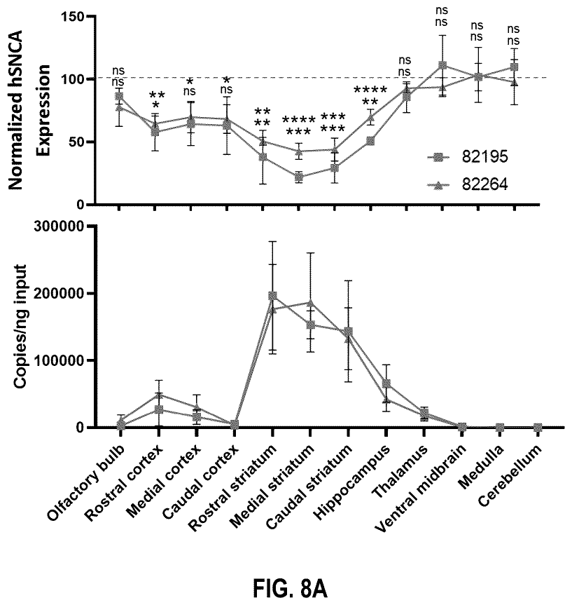

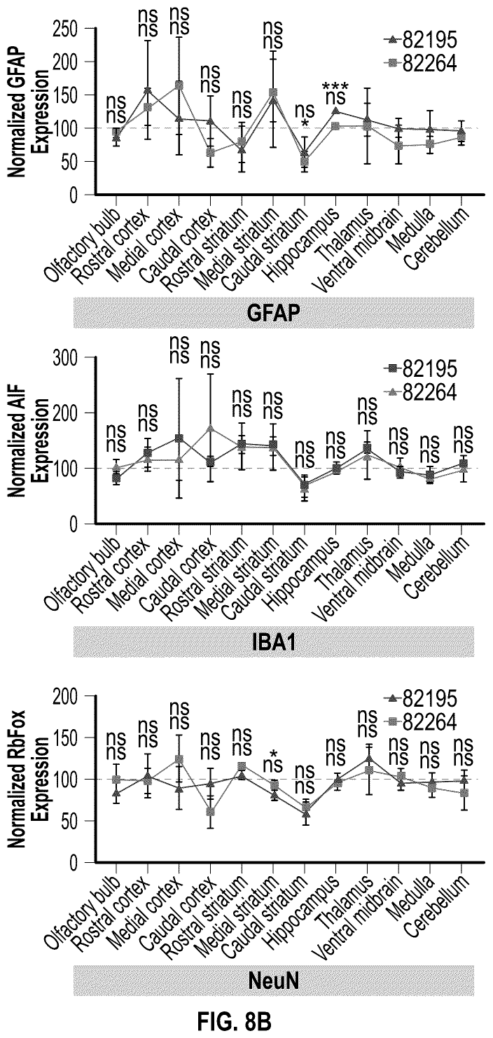

Alpha-synuclein, ZFP-TF, GFAP, IBA1, and NeuN mRNA expression data from the experiment are shown in FIGS. 8A and 8B. The data show that there was repression of alpha-synuclein in brain regions with significant ZFP-TF expression. Furthermore, the GFAP, IBA1, and NeuN expression data indicate that the ZFP-TFs were well-tolerated, as their administration did not lead to elevated expression of markers of neuroinflammation or reduced expression of the neuronal marker NeuN.

### List of Sequences

Table 1 below lists 47 exemplary engineered ZFPs of the present disclosure. For each ZFP, the genomic target sequence (Binding Sequence) and the DNA-binding recognition helix sequences (i.e., F1-F6) of each zinc finger within the ZFP domain are shown in a single row. “\hat{ }” in below table indicates that the arginine (R) residue at the 4th position upstream of the 1st amino acid in the indicated helix is changed to glutamine (Q). The SEQ ID NO for each sequence is shown in parenthesis underneath the sequence. In the nucleotide sequences in column 2, nucleotides contacted by the ZFP are shown in capital letters.

TABLE 1
Exemplary ZFPs

| | Binding | | | | | | |
|---|---|---|---|---|---|---|---|
| | Sequence | | | | | | |
| ZFP | (capital | | | | | | |
| name | letters) | F1 | F2 | F3 | F4 | F5 | F6 |
| SBS- | aTCCGAGATAG | QSGHLAR | QRKDLTQ | RSDHLSR | TRDGLRQ | RSANLAR | DSSDRKK |
| 82090 | GGaCGAGGAg | (20) | (21) | (22) | (23) | (24) | (25) |
| | (19) | | | | | | |
| SBS- | CCGAGATAGGG | RSDNLST | RSDNLTR | DRSDLSR | RREHLRA | TSGNLTR | QRSHLSD |
| 82092 | ACGAGGAGc | (27) | (28) | (29) | (30) | (31) | (32) |
| | (26) | | | | | | |
| SBS- | CAGGGAAaGCA | ERGTLAR | MACCRYA | RSADLTR | QSGDLTR | QSGNLAR | RQEHRVA |
| 82107 | GCGAGCGCCg | (34) | (35) 5 | (36) | (37) | (38) | (39) |
| | (33) | | | | | | |
| SBS- | gAAAGCAGCGA | RSAHLSR | DRSDLSR | MSCCRSA | RSADLSR | QSGDLTR | QRSNLNI |
| 82110 | GCGCCGGGa | (41) | (29) | (42) | (43) | (37) | (44) |
| | (40) | | | | | | |
| SBS- | gGACGGCGACG | RSDNLSV | ASWTLTQ | DRSDLSR | DRSNLTR | DRSHLTR | DRSNLTR |
| 82136 | ACCAGAAGg | (46) | (47) | (29) | (48) | (49) | (48) |
| | (45) | | | | | | |
| SBS- | CGCGACGCGGA | LRHHLTR | VAEYRYK | RSDNLSV | RHSHITS | RSDTLSA | RSADLSR |
| 82148 | AGTGAGGTg | (51) | (52) | (46) | (53) | (54) | (43) |
| | (50) | | | | | | |
| SBS- | CGCTCACGAGG | RSDNLST | QSGHLSR | DAHHLHR | RSANLTR | DRSNRTT | HRSSLRR |
| 82190 | GTGGAAAGg | (27) | (56) | (57) | (58) | (59) | (60) |
| | (55) | | | | | | |
| SBS- | aTTCGACGACA | RSDDLTR | QKFPRDS | RSDHLTQ | DSANLSR | DRSNLSR | DRTSLKW |
| 82195 | GGTTAGCGg | (62) | (63) | (64) | (65) | (66) | (67) |
| | (61) | | | | | | |
| SBS- | gGCCGCTGTGA | DRSNLSR | HLCCLGR | RSCCLHL | RNASRTR | QSSDLSR | HRKSLSR |
| 82208 | GCcGGCGACg | (66) | (69) | (70) | (71) | (72) | (73) |
| | (68) | | | | | | |
| SBS- | aGGCGGCACCC | DRSNLSR | DRSHLAR | RSDHLSE | TSSDRTK | DRSHLSR | DRSHLAR |
| 82215 | GGgGGGGACt | (66) | (75) | (76) | (77) | (78) | (75) |
| | (74) | | | | | | |
| SBS- | gAGCGCACAGG | DRSHLTR | DRSHLAR | QSGNLAR | AKWNLDA | QSGDLTR | TQGYLRK |
| 82225 | AAgGGCGGAg | (49) | (75) | (38) | (80) | (37) | (81) |
| | (79) | | | | | | |
| SBS- | gGCGGGGaGGA | TSGSLSR | QAGHLAK | DRSARTR | QSGHLSR | RSAHLSR | QSSDLRR |
| 82242 | GTCGGAGTTg | (83) | (84) | (85) | (56) | (41) | (86) |
| | (82) | | | | | | |
| SBS- | gGTCGTGgGCA | RSDHLSR | QHAHRNT | RPDTLRD | QNATRTK | RSDALSR | DRSALAR |
| 82294 | CCGGGAGGGg | (22) | (88) | (89) | (90) | (91) | (92) |
| | (87) | | | | | | |
| SBS- | gGCTGCTTCTC | NNRDLIN | TSSNLSR | RSDTLSE | NNRDRTK | QSSDLSR | HRSTRNR |
| 82329 | CGgGATCCGc | (94) | (95) | (96) | (97) | (72) | (98) |
| | (93) | | | | | | |
| SBS- | gGAGGACGGCG | QNAHRKT | DNSTRIK | DRSNLSR | HLCCLGR | DRSNLSR | TSANLSR |
| 82135 | ACgACCAGAa | (100) | (101) | (66) | (69) | (66) | (102) |
| | (99) | | | | | | |
| SBS- | aGAGGGGGCGA | QRNHRTT | DRSNLTR | MSCCRSA | RSADLSR | RSAHLSR | TSANLSR |
| 82144 | GCGACCGAg | (104) | (48) | (42) | (43) | (41) | (102) |
| | (103) | | | | | | |
| SBS- | CGTCGCGGCGC | QSSDLSR | DRSALAR | DRSYRNT | RRSDLKR | RSADLTR | DRSALAR |
| 82150 | TCgGTCGCTc | (72) | (92) | (106) | (107) | (36) | (92) |
| | (105) | | | | | | |
| SBS- | CTGCGCTGCAG | DSSTRKT | QSATRTK | ERGTLAR | QSGDLTR | QSSDLSR | AASNRSK |
| 82158 | CCCGCACGCa | (109) | (110) | (34) | (37) | (72) | (111) |
| | (108) | | | | | | |
| SBS- | CGTCGAAtGGC | RSDNLSQ | ASNDRKK | YDYGRYT | DRSHLAR | QSGNLAR | DRSALAR |
| 82197 | CACTCCCAGt | (113) | (114) | (115) | (75) | (38) | (92) |
| | (112) | | | | | | |
| SBS- | gTACCTGtGGA | RSDHLSE | QSNHRKT | VYEGLKK | QSGHLSR | RSDVLSE | DRSNRIK |
| 82264 | TCTAAACGGg | (76) | (117) | (118) | (56) | (119) | (120) |
| | (116) | | | | | | |
| SBS- | aAAGTGGgGGC | ^{∧}RRADLSR | YHWYLKK | ^{∧}QSSDLSR | DRSHLAR | ^{∧}RSDHLSQ | TSANRTT |
| 84948 | GCTTTTGCGc | (122) | (123) | (72) | (75) | (124) | (125) |
| | (121) | | | | | | |
| SBS- | gCAAGGCGGGA | ^{∧}RSDHLST | RSDALAR | ^{∧}QSANRTT | RSAHLSR | DRSHLTR | QSGNLHV |
| 84953 | AAGTGGGGg | (127) | (128) | (129) | (41) | (49) | (130) |
| | (126) | | | | | | |
| SBS- | gCAAGGCGGGA | ^{∧}RSDHLST | RSDALAR | ^{∧}QSANRTT | RSAHLSR | ^{∧}DRSHLTR | QSGNLHV |
| 84954 | AAGTGGGGg | (127) | (128) | (129) | (41) | (49) | (130) |
| | (126) | | | | | | |
| SBS- | gCGCAAGgCGG | ^{∧}RSDHLSE | AKHHRKT | QSGNLAR | RQEHRVA | RSDNLSA | QNRDRKN |
| 84958 | GAAAGTGGGg | (76) | (132) | (38) | (39) | (133) | (134) |
| | (131) | | | | | | |
| SBS- | CGCGCAAGGCG | ^{∧}RSDSLSV | QNANRKT | RSAHLSR | DRSHLAR | QSANRTT | RRSDLKR |
| 84961 | GGAAAGTGg | (136) | (137) | (41) | (75) | (129) | (107) |
| | (135) | | | | | | |
| SBS- | CGCGCAAGGCG | ^{∧}RSDSLSV | QNANRKT | ^{∧}RSAHLSR | DRSHLAR | QSANRTT | RRSDLKR |
| 84962 | GGAAAGTGg | (136) | (137) | (41) | (75) | (129) | (107) |
| | (135) | | | | | | |
| SBS- | gGAGCACGCTG | ^{∧}QSANRTT | RSAHLSR | ^{∧}QSGDLTR | QSSDLRR | DQSNLRA | RSANLTR |
| 84998 | CAGGGAAAg | (129) | (41) | (37) | (86) | (139) | (58) |
| | (138) | | | | | | |
| SBS- | gGAGCACGCTG | ^{∧}QSANRTT | RSAHLSR | ^{∧}QSGDLTR | QSSDLRR | ^{∧}DQSNLRA | RSANLTR |
| 84999 | CAGGGAAAg | (129) | (41) | (37) | (86) | (139) | (58) |
| | (138) | | | | | | |
| SBS- | aGCACGCtGCA | ^{∧}QSGDLTR | QRSNLNI | RSAHLSR | QSGDLTR | DSSTRKT | QSATRTK |
| 85000 | GGGAAAGCAg | (37) | (44) | (41) | (37) | (109) | (110) |
| | (140) | | | | | | |
| SBS- | aGCACGCtGCA | ^{∧}QSGDLTR | QRSNLNI | ^{∧}RSAHLSR | QSGDLTR | DSSTRKT | QSATRTK |
| 85001 | GGGAAAGCAg | (37) | (44) | (41) | (37) | (109) | (110) |
| | (140) | | | | | | |
| SBS- | tGCAGGGAAAG | ^{∧}MSCCRSA | RSADLSR | ^{∧}QSGDLTR | QRSNLNI | ^{∧}RSAHLSR | QSGDLTR |
| 85014 | CAGCGAGCg | (42) | (43) | (37) | (44) | (41) | (37) |
| | (141) | | | | | | |
| SBS- | gTGGGGGCGGA | ^{∧}RSDNLAR | DRSVLHR | QSAHRKN | RHSHLTS | RSDHLSE | RNDHRTT |
| 85042 | GAGCCGAGg | (143) | (144) | (145) | (53) | (76) | (146) |
| | (142) | | | | | | |
| SBS- | gAGAGGGgGCG | ^{∧}QRNHRTT | DRSNLTR | MSCCRSA | RSADLSR | RSDHLSR | QGAHLGA |
| 85069 | AGCGACCGAg | (104) | (48) | (42) | (43) | (22) | (148) |
| | (147) | | | | | | |
| SBS- | gcAGCGCAGAC | ^{∧}RSDALSV | DSSHRTR | DNRDLST | DRSNLTR | QSGDLTR | TQGYLRK |
| 85096 | CCCGGCCCGg | (150) | (151) | (152) | (48) | (37) | (81) |
| | (149) | | | | | | |
| SBS- | tCTCGGAGGGG | ^{∧}RSDHLSQ | DNSHRTR | DRSDLSR | RSTHLVR | DRSHLTR | DRSYRNT |
| 85102 | CCgGGCCGGg | (124) | (154) | (29) | (155) | (49) | (106) |
| | (153) | | | | | | |
| SBS- | gGACGCTCTCG | ^{∧}DRSDLSR | RSTHLVR | DRSHLTR | DRSYRNT | QESDLVR | DRSNLTR |
| 85105 | GAGGGGCCg | (29) | (155) | (49) | (106) | (157) | (48) |
| | (156) | | | | | | |
| SBS- | aAAAGGAGCGC | ^{∧}RSDNLSV | RRAHLNQ | DQSNLRA | RRSDLKR | QSGHLAR | QRVALQA |
| 85177 | ACAGGAAGg | (46) | (159) | (139) | (107) | (20) | (160) |
| | (158) | | | | | | |
| SBS- | gGAAAAGgAGC | ^{∧}QSGNLAR | AKWNLDA | QSGDLTR | TQGYLRK | RSDNLSV | QSGDLTR |
| 85180 | GCACAGGAAg | (38) | (80) | (37) | (81) | (46) | (37) |
| | (161) | | | | | | |
| SBS- | tCAGGTAaGTA | ^{∧}TSGNLTR | RSELLQS | ARSTRIT | QSGSLTR | QSGSLTR | AKWNLDA |
| 85216 | CCTGTGGATc | (31) | (163) | (164) | (165) | (165) | (80) |
| | (162) | | | | | | |
| SBS- | tCAGGTAaGTA | ^{∧}TSGNLTR | RSELLQS | ^{∧}ARSTRIT | QSGSLTR | QSGSLTR | AKWNLDA |
| 85217 | CCTGTGGATc | (31) | (163) | (164) | (165) | (165) | (80) |
| | (162) | | | | | | |
| SBS- | tCAGGTAaGTA | ^{∧}TSGNLTR | RSELLQS | ^{∧}ARSTRIT | QSGSLTR | ^{∧}QSGSLTR | AKWNLDA |
| 85218 | CCTGTGGATc | (31) | (163) | (164) | (165) | (165) | (80) |
| | (162) | | | | | | |
| SBS- | tCTGGGAGGTG | ^{∧}RPYTLRL | DNSNRIK | ^{∧}RSANLAR | TSGHLSR | QSGHLAR | RKWTLQG |
| 85256 | AGTACTTGt | (167) | (168) | (24) | 169) | (20) | (170) |
| | (166) | | | | | | |
| SBS- | tCTGGGAGGTG | ^{∧}RPYTLRL | DNSNRIK | ^{∧}RSANLAR | TSGHLSR | ^{∧}QSGHLAR | RKWTLQG |
| 85257 | AGTACTTGt | (167) | (168) | (24) | (169) | (20) | (170) |
| | (166) | | | | | | |
| SBS- | CCAAAGGGACA | ^{∧}QSADRTK | DNSNRIK | ^{∧}RSDNLST | DSANRIK | ^{∧}RSDHLSE | QNANRIT |
| 85260 | AGTACTCAc | (172) | (168) | (27) | (173) | (76) | (174) |
| | (171) | | | | | | |
| SBS- | CCCAAAGgGAC | ^{∧}QSADRTK | DNSNRIK | RSANLSV | DRANLSR | RSDNLSV | QKATRIT |
| 85261 | AAGTACTCAc | (172) | (168) | (176) | (177) | (46) | (178) |
| | (175) | | | | | | |
| SBS- | CCCAAAGgGAC | ^{∧}QSADRTK | DNSNRIK | ^{∧}RSANLSV | DRANLSR | RSDNLSV | QKATRIT |
| 85262 | AAGTACTCAc | (172) | (168) | (176) | (177) | (46) | (178) |
| | (175) | | | | | | |
| SBS- | CCCAAAGgGAC | ^{∧}QSADRTK | DNSNRIK | ^{∧}RSANLSV | DRANLSR | ^{∧}RSDNLSV | QKATRIT |
| 85263 | AAGTACTCAc | (172) | (168) | (176) | (177) | (46) | (178) |
| | (175) | | | | | | |

Table 2 below lists the full amino acid sequences of 47 exemplary ZFP-TFs of the present disclosure, wherein the DNA-binding recognition helix sequences are in boldface, intramodule and intermodule linkers are underlined. The R(−5)Q backbone mutations are indicated by boldface and underline. The linkers between the ZFP and KRAB domain and double-underlined.

TABLE 2
Exemplary ZFP-TF Sequences

| ZFP-TF | |
|---|---|
| name | Amino Acid Sequences |
| SBS- | MAPKKKRKVGVPAAMAERPFQCRICMRNFSQSGHLARHIRTHTGEKPFAC |
| 82090 | DICGRKFAQRKDLTQHTKIHTHPRAPIPKPFQCRICMRNFSRSDHLSRHI |
| | RTHTGEKPFACDICGRKFATRDGLRQHTKIHTGSQKPFQCRICMRNFSRS |
| | ANLARHIRTHTGEKPFACDICGRKFADSSDRKKHTKIHLRQKDAARGSGG |
| | DAKSLTAWSRTLVTFKDVFVDFTREEWKLLDTAQQIVYRNVMLENYKNLV |
| | SLGYQLTKPDVILRLEKGEEPWLVEREIHQETHPDSETAFEIKSSVRS |
| | (SEQ ID NO: 179) |
| SBS- | MAPKKKRKVGVPAAMAERPFQCRICMRNFSRSDNLSTHIRTHTGEKPFAC |
| 82092 | DICGRKFARSDNLTRHTKIHTGSQKPFQCRICMRNFSDRSDLSRHIRTHT |
| | GEKPFACDICGRKFARREHLRAHTKIHTGSQKPFQCRICMRNFSTSGNLT |
| | RHIRTHTGEKPFACDICGRKFAQRSHLSDHTKIHLRQKDAARGSGGDAKS |
| | LTAWSRTLVTFKDVFVDFTREEWKLLDTAQQIVYRNVMLENYKNLVSLGY |
| | QLTKPDVILRLEKGEEPWLVEREIHQETHPDSETAFEIKSSVRS (SEQ |
| | ID NO: 180) |
| SBS- | MAPKKKRKVGVPAAMAERPFQCRICMRNFSERGTLARHIRTHTGEKPFAC |
| 82107 | DICGRKFAMACCRYAHTKIHTGSQKPFQCRICMRNFSRSADLTRHIRTHT |
| | GEKPFACDICGRKFAQSGDLTRHTKIHTHPRAPIPKPFQCRICMRNFSQS |
| | GNLARHIRTHTGEKPFACDICGRKFARQEHRVAHTKIHLRQKDAARGSGG |
| | DAKSLTAWSRTLVTFKDVFVDFTREEWKLLDTAQQIVYRNVMLENYKNLV |
| | SLGYQLTKPDVILRLEKGEEPWLVEREIHQETHPDSETAFEIKSSVRS |
| | (SEQ ID NO: 181) |
| SBS- | MAPKKKRKVGVPAAMAERPFQCRICMRNFSRSAHLSRHIRTHTGEKPFAC |
| 82110 | DICGRKFADRSDLSRHTKIHTGSQKPFQCRICMRNFSMSCCRSAHIRTHT |
| | GEKPFACDICGRKFARSADLSRHTKIHTGSQKPFQCRICMRNFSQSGDLT |
| | RHIRTHTGEKPFACDICGRKFAQRSNLNIHTKIHLRQKDAARGSGGDAKS |
| | LTAWSRTLVTFKDVFVDFTREEWKLLDTAQQIVYRNVMLENYKNLVSLGY |
| | QLTKPDVILRLEKGEEPWLVEREIHQETHPDSETAFEIKSSVRS (SEQ |
| | ID NO: 182) |
| SBS- | MAPKKKRKVGVPAAMAERPFQCRICMRNFSRSDNLSVHIRTHTGEKPFAC |
| 82136 | DICGRKFAASWTLTQHTKIHTGSQKPFQCRICMRNFSDRSDLSRHIRTHT |
| | GEKPFACDICGRKFADRSNLTRHTKIHTGSQKPFQCRICMRNFSDRSHLT |
| | RHIRTHTGEKPFACDICGRKFADRSNLTRHTKIHLRQKDAARGSGGDAKS |
| | LTAWSRTLVTFKDVFVDFTREEWKLLDTAQQIVYRNVMLENYKNLVSLGY |
| | QLTKPDVILRLEKGEEPWLVEREIHQETHPDSETAFEIKSSVRS (SEQ |
| | ID NO: 183) |
| SBS- | MAPKKKRKVGVPAAMAERPFQCRICMRNFSLRHHLTRHIRTHTGEKPFAC |
| 82148 | DICGRKFAVAEYRYKHTKIHTGSQKPFQCRICMRNFSRSDNLSVHIRTHT |
| | GEKPFACDICGRKFARHSHLTSHTKIHTGSQKPFQCRICMRNFSRSDTLS |
| | AHIRTHTGEKPFACDICGRKFARSADLSRHTKIHLRQKDAARGSGGDAKS |
| | LTAWSRTLVTFKDVFVDFTREEWKLLDTAQQIVYRNVMLENYKNLVSLGY |
| | QLTKPDVILRLEKGEEPWLVEREIHQETHPDSETAFEIKSSVRS (SEQ |
| | ID NO: 184) |
| SBS- | MAPKKKRKVGVPAAMAERPFQCRICMRNFSRSDNLSTHIRTHTGEKPFAC |
| 82190 | DICGRKFAQSGHLSRHTKIHTGSQKPFQCRICMRNFSDAHHLHRHIRTHT |
| | GEKPFACDICGRKFARSANLTRHTKIHTGSQKPFQCRICMRNFSDRSNRT |
| | THIRTHTGEKPFACDICGRKFAHRSSLRRHTKIHLRQKDAARGSGGDAKS |
| | LTAWSRTLVTFKDVFVDFTREEWKLLDTAQQIVYRNVMLENYKNLVSLGY |
| | QLTKPDVILRLEKGEEPWLVEREIHQETHPDSETAFEIKSSVRS (SEQ |
| | ID NO: 185) |
| SBS- | MAPKKKRKVGVPAAMAERPFQCRICMRNFSRSDDLTRHIRTHTGEKPFAC |
| 82195 | DICGRKFAQKFPRDSHTKIHTGSQKPFQCRICMRNFSRSDHLTQHIRTHT |
| | GEKPFACDICGRKFADSANLSRHTKIHTGSQKPFQCRICMRNFSDRSNLS |
| | RHIRTHTGEKPFACDICGRKFADRTSLKWHTKIHLRQKDAARGSGGDAKS |
| | LTAWSRTLVTFKDVFVDFTREEWKLLDTAQQIVYRNVMLENYKNLVSLGY |
| | QLTKPDVILRLEKGEEPWLVEREIHQETHPDSETAFEIKSSVRS (SEQ |
| | ID NO: 186) |
| SBS- | MAPKKKRKVGVPAAMAERPFQCRICMRNFSDRSNLSRHIRTHTGEKPFAC |
| 82208 | DICGRKFAHLCCLGRHTKIHTHPRAPIPKPFQCRICMRNFSRSCCLHLHI |
| | RTHTGEKPFACDICGRKFARNASRTRHTKIHTGSQKPFQCRICMRNFSQS |
| | SDLSRHIRTHTGEKPFACDICGRKFAHRKSLSRHTKIHLRQKDAARGSGG |
| | DAKSLTAWSRTLVTFKDVFVDFTREEWKLLDTAQQIVYRNVMLENYKNLV |
| | SLGYQLTKPDVILRLEKGEEPWLVEREIHQETHPDSETAFEIKSSVRS |
| | (SEQ ID NO: 187) |
| SBS- | MAPKKKRKVGVPAAMAERPFQCRICMRNFSDRSNLSRHIRTHTGEKPFAC |
| 82215 | DICGRKFADRSHLARHTKIHTHPRAPIPKPFQCRICMRNFSRSDHLSEHI |
| | RTHTGEKPFACDICGRKFATSSDRTKHTKIHTGSQKPFQCRICMRNFSDR |
| | SHLSRHIRTHTGEKPFACDICGRKFADRSHLARHTKIHLRQKDAARGSGG |
| | DAKSLTAWSRTLVTFKDVFVDFTREEWKLLDTAQQIVYRNVMLENYKNLV |
| | SLGYQLTKPDVILRLEKGEEPWLVEREIHQETHPDSETAFEIKSSVRS |
| | (SEQ ID NO: 188) |
| SBS- | MAPKKKRKVGVPAAMAERPFQCRICMRNFSDRSHLTRHIRTHTGEKPFAC |
| 82225 | DICGRKFADRSHLARHTKIHTHPRAPIPKPFQCRICMRNFSQSGNLARHI |
| | RTHTGEKPFACDICGRKFAAKWNLDAHTKIHTGSQKPFQCRICMRNFSQS |
| | GDLTRHIRTHTGEKPFACDICGRKFATQGYLRKHTKIHLRQKDAARGSGG |
| | DAKSLTAWSRTLVTFKDVFVDFTREEWKLLDTAQQIVYRNVMLENYKNLV |
| | SLGYQLTKPDVILRLEKGEEPWLVEREIHQETHPDSETAFEIKSSVRS |
| | (SEQ ID NO: 189) |
| SBS- | MAPKKKRKVGVPAAMAERPFQCRICMRNFSTSGSLSRHIRTHTGEKPFAC |
| 82242 | DICGRKFAQAGHLAKHTKIHTGSQKPFQCRICMRNFSDRSARTRHIRTHT |
| | GEKPFACDICGRKFAQSGHLSRHTKIHTHPRAPIPKPFQCRICMRNFSRS |
| | AHLSRHIRTHTGEKPFACDICGRKFAQSSDLRRHTKIHLRQKDAARGSGG |
| | DAKSLTAWSRTLVTFKDVFVDFTREEWKLLDTAQQIVYRNVMLENYKNLV |
| | SLGYQLTKPDVILRLEKGEEPWLVEREIHQETHPDSETAFEIKSSVRS |
| | (SEQ ID NO: 190) |
| SBS- | MAPKKKRKVGVPAAMAERPFQCRICMRNFSRSDHLSRHIRTHTGEKPFAC |
| 82294 | DICGRKFAQHAHRNTHTKIHTGSQKPFQCRICMRNFSRPDTLRDHIRTHT |
| | GEKPFACDICGRKFAQNATRTKHTKIHTHPRAPIPKPFQCRICMRNFSRS |
| | DALSRHIRTHTGEKPFACDICGRKFADRSALARHTKIHLRQKDAARGSGG |
| | DAKSLTAWSRTLVTFKDVFVDFTREEWKLLDTAQQIVYRNVMLENYKNLV |
| | SLGYQLTKPDVILRLEKGEEPWLVEREIHQETHPDSETAFEIKSSVRS |
| | (SEQ ID NO: 191) |
| SBS- | MAPKKKRKVGVPAAMAERPFQCRICMRNFSNNRDLINHIRTHTGEKPFAC |
| 82329 | DICGRKFATSSNLSRHTKIHTHPRAPIPKPFQCRICMRNFSRSDTLSEHI |
| | RTHTGEKPFACDICGRKFANNRDRTKHTKIHTGSQKPFQCRICMRNFSQS |
| | SDLSRHIRTHTGEKPFACDICGRKFAHRSTRNRHTKIHLRQKDAARGSGG |
| | DAKSLTAWSRTLVTFKDVFVDFTREEWKLLDTAQQIVYRNVMLENYKNLV |
| | SLGYQLTKPDVILRLEKGEEPWLVEREIHQETHPDSETAFEIKSSVRS |
| | (SEQ ID NO: 192) |
| SBS- | MAPKKKRKVGVPAAMAERPFQCRICMRNFSQNAHRKTHIRTHTGEKPFAC |
| 82135 | DICGRKFADNSTRIKHTKIHTHPRAPIPKPFQCRICMRNFSDRSNLSRHI |
| | RTHTGEKPFACDICGRKFAHLCCLGRHTKIHTGSQKPFQCRICMRNFSDR |
| | SNLSRHIRTHTGEKPFACDICGRKFATSANLSRHTKIHLRQKDAARGSGG |
| | DAKSLTAWSRTLVTFKDVFVDFTREEWKLLDTAQQIVYRNVMLENYKNLV |
| | SLGYQLTKPDVILRLEKGEEPWLVEREIHQETHPDSETAFEIKSSVRS |
| | (SEQ ID NO: 193) |
| SBS- | MAPKKKRKVGVPAAMAERPFQCRICMRNFSQRNHRTTHIRTHTGEKPFAC |
| 82144 | DICGRKFADRSNLTRHTKIHTGSQKPFQCRICMRNFSMSCCRSAHIRTHT |
| | GEKPFACDICGRKFARSADLSRHTKIHTGSQKPFQCRICMRNFSRSAHLS |
| | RHIRTHTGEKPFACDICGRKFATSANLSRHTKIHLRQKDAARGSGGDAKS |
| | LTAWSRTLVTFKDVFVDFTREEWKLLDTAQQIVYRNVMLENYKNLVSLGY |
| | QLTKPDVILRLEKGEEPWLVEREIHQETHPDSETAFEIKSSVRS (SEQ |
| | ID NO: 194) |
| SBS- | MAPKKKRKVGVPAAMAERPFQCRICMRNFSQSSDLSRHIRTHTGEKPFAC |
| 82150 | DICGRKFADRSALARHTKIHTHPRAPIPKPFQCRICMRNFSDRSYRNTHI |
| | RTHTGEKPFACDICGRKFARRSDLKRHTKIHTGSQKPFQCRICMRNFSRS |
| | ADLTRHIRTHTGEKPFACDICGRKFADRSALARHTKIHLRQKDAARGSGG |
| | DAKSLTAWSRTLVTFKDVFVDFTREEWKLLDTAQQIVYRNVMLENYKNLV |
| | SLGYQLTKPDVILRLEKGEEPWLVEREIHQETHPDSETAFEIKSSVRS |
| | (SEQ ID NO: 195) |
| SBS- | MAPKKKRKVGVPAAMAERPFQCRICMRNFSDSSTRKTHIRTHTGEKPFAC |
| 82158 | DICGRKFAQSATRTKHTKIHTHPRAPIPKPFQCRICMRNFSERGTLARHI |
| | RTHTGEKPFACDICGRKFAQSGDLTRHTKIHTGSQKPFQCRICMRNFSQS |
| | SDLSRHIRTHTGEKPFACDICGRKFAAASNRSKHTKIHLRQKDAARGSGG |
| | DAKSLTAWSRTLVTFKDVFVDFTREEWKLLDTAQQIVYRNVMLENYKNLV |
| | SLGYQLTKPDVILRLEKGEEPWLVEREIHQETHPDSETAFEIKSSVRS |
| | (SEQ ID NO: 196) |
| SBS- | MAPKKKRKVGVPAAMAERPFQCRICMRNFSRSDNLSQHIRTHTGEKPFAC |
| 82197 | DICGRKFAASNDRKKHTKIHTGSQKPFQCRICMRNFSYDYGRYTHIRTHT |
| | GEKPFACDICGRKFADRSHLARHTKIHTHPRAPIPKPFQCRICMRNFSQS |
| | GNLARHIRTHTGEKPFACDICGRKFADRSALARHTKIHLRQKDAARGSGG |
| | DAKSLTAWSRTLVTFKDVFVDFTREEWKLLDTAQQIVYRNVMLENYKNLV |
| | SLGYQLTKPDVILRLEKGEEPWLVEREIHQETHPDSETAFEIKSSVRS |
| | (SEQ ID NO: 197) |
| SBS- | MAPKKKRKVGVPAAMAERPFQCRICMRNFSRSDHLSEHIRTHTGEKPFAC |
| 82264 | DICGRKFAQSNHRKTHTKIHTGSQKPFQCRICMRNFSVYEGLKKHIRTHT |
| | GEKPFACDICGRKFAQSGHLSRHTKIHTHPRAPIPKPFQCRICMRNFSRS |
| | DVLSEHIRTHTGEKPFACDICGRKFADRSNRIKHTKIHLRQKDAARGSGG |
| | DAKSLTAWSRTLVTFKDVFVDFTREEWKLLDTAQQIVYRNVMLENYKNLV |
| | SLGYQLTKPDVILRLEKGEEPWLVEREIHQETHPDSETAFEIKSSVRS |
| | (SEQ ID NO: 198) |
| SBS- | MAPKKKRKVGVPAAMAERPFQCRICM [symbol] NFSRRADLSRHIRTHTGEKPFAC |
| 84948 | DICGRKFAYHWYLKKHTKIHTGSQKPFQCRICM [symbol] NFSQSSDLSRHIRTHT |
| | GEKPFACDICGRKFADRSHLARHTKIHTHPRAPIPKPFQCRICM [symbol] NFSRS |
| | DHLSQHIRTHTGEKPFACDICGRKFATSANRTTHTKIHLRQKDAARGSGG |
| | DAKSLTAWSRTLVTFKDVFVDFTREEWKLLDTAQQIVYRNVMLENYKNLV |
| | SLGYQLTKPDVILRLEKGEEPWLVEREIHQETHPDSETAFEIKSSVRS |
| | (SEQ ID NO: 199) |
| SBS- | MAPKKKRKVGVPAAMAERPFQCRICM [symbol] NFSRSDHLSTHIRTHTGEKPFAC |
| 84953 | DICGRKFARSDALARHTKIHTGSQKPFQCRICM [symbol] NFSQSANRTTHIRTHT |
| | GEKPFACDICGRKFARSAHLSRHTKIHTGSQKPFQCRICMRNFSDRSHLT |
| | RHIRTHTGEKPFACDICGRKFAQSGNLHVHTKIHLRQKDAARGSGGDAKS |
| | LTAWSRTLVTFKDVFVDFTREEWKLLDTAQQIVYRNVMLENYKNLVSLGY |
| | QLTKPDVILRLEKGEEPWLVEREIHQETHPDSETAFEIKSSVRS (SEQ |
| | ID NO: 200) |
| SBS- | MAPKKKRKVGVPAAMAERPFQCRICM [symbol] NFSRSDHLSTHIRTHTGEKPFAC |
| 84954 | DICGRKFARSDALARHTKIHTGSQKPFQCRICM [symbol] NFSQSANRTTHIRTHT |
| | GEKPFACDICGRKFARSAHLSRHTKIHTGSQKPFQCRICM [symbol] NFSDRSHLT |
| | RHIRTHTGEKPFACDICGRKFAQSGNLHVHTKIHLRQKDAARGSGGDAKS |
| | LTAWSRTLVTFKDVFVDFTREEWKLLDTAQQIVYRNVMLENYKNLVSLGY |
| | QLTKPDVILRLEKGEEPWLVEREIHQETHPDSETAFEIKSSVRS (SEQ |
| | ID NO: 201) |
| SBS- | MAPKKKRKVGVPAAMAERPFQCRICM [symbol] NFSRSDHLSEHIRTHTGEKPFAC |
| 84958 | DICGRKFAAKHHRKTHTKIHTGSQKPFQCRICMRNFSQSGNLARHIRTHT |
| | GEKPFACDICGRKFARQEHRVAHTKIHTHPRAPIPKPFQCRICMRNFSRS |
| | DNLSAHIRTHTGEKPFACDICGRKFAQNRDRKNHTKIHLRQKDAARGSGG |
| | DAKSLTAWSRTLVTFKDVFVDFTREEWKLLDTAQQIVYRNVMLENYKNLV |
| | SLGYQLTKPDVILRLEKGEEPWLVEREIHQETHPDSETAFEIKSSVRS |
| | (SEQ ID NO: 202) |
| SBS- | MAPKKKRKVGVPAAMAERPFQCRICM [symbol] NFSRSDSLSVHIRTHTGEKPFAC |
| 84961 | DICGRKFAQNANRKTHTKIHTGSQKPFQCRICMRNFSRSAHLSRHIRTHT |
| | GEKPFACDICGRKFADRSHLARHTKIHTGSQKPFQCRICMRNFSQSANRT |
| | THIRTHTGEKPFACDICGRKFARRSDLKRHTKIHLRQKDAARGSGGDAKS |
| | LTAWSRTLVTFKDVFVDFTREEWKLLDTAQQIVYRNVMLENYKNLVSLGY |
| | QLTKPDVILRLEKGEEPWLVEREIHQETHPDSETAFEIKSSVRS (SEQ |
| | ID NO: 203) |
| SBS- | MAPKKKRKVGVPAAMAERPFQCRICMQNFSRSDSLSVHIRTHTGEKPFAC |
| 84962 | DICGRKFAQNANRKTHTKIHTGSQKPFQCRICM [symbol] NFSRSAHLSRHIRTHT |
| | GEKPFACDICGRKFADRSHLARHTKIHTGSQKPFQCRICMRNFSQSANRT |
| | THIRTHTGEKPFACDICGRKFARRSDLKRHTKIHLRQKDAARGSGGDAKS |
| | LTAWSRTLVTFKDVFVDFTREEWKLLDTAQQIVYRNVMLENYKNLVSLGY |
| | QLTKPDVILRLEKGEEPWLVEREIHQETHPDSETAFEIKSSVRS (SEQ |
| | ID NO: 204) |
| SBS- | MAPKKKRKVGVPAAMAERPFQCRICMQNFSQSANRTTHIRTHTGEKPFAC |
| 84998 | DICGRKFARSAHLSRHTKIHTGSQKPFQCRICMQNFSQSGDLTRHIRTHT |
| | GEKPFACDICGRKFAQSSDLRRHTKIHTGSQKPFQCRICMRNFSDQSNLR |
| | AHIRTHTGEKPFACDICGRKFARSANLTRHTKIHLRQKDAARGSGGDAKS |
| | LTAWSRTLVTFKDVFVDFTREEWKLLDTAQQIVYRNVMLENYKNLVSLGY |
| | QLTKPDVILRLEKGEEPWLVEREIHQETHPDSETAFEIKSSVRS (SEQ |
| | ID NO: 205) |
| SBS- | MAPKKKRKVGVPAAMAERPFQCRICM [symbol] NFSQSANRTTHIRTHTGEKPFAC |
| 84999 | DICGRKFARSAHLSRHTKIHTGSQKPFQCRICM [symbol] NFSQSGDLTRHIRTHT |
| | GEKPFACDICGRKFAQSSDLRRHTKIHTGSQKPFQCRICM [symbol] NFSDQSNLR |
| | AHIRTHTGEKPFACDICGRKFARSANLTRHTKIHLRQKDAARGSGGDAKS |
| | LTAWSRTLVTFKDVFVDFTREEWKLLDTAQQIVYRNVMLENYKNLVSLGY |
| | QLTKPDVILRLEKGEEPWLVEREIHQETHPDSETAFEIKSSVRS (SEQ |
| | ID NO: 206) |
| SBS- | MAPKKKRKVGVPAAMAERPFQCRICM [symbol] NFSQSGDLTRHIRTHTGEKPFAC |
| 85000 | DICGRKFAQRSNLNIHTKIHTGSQKPFQCRICMRNFSRSAHLSRHIRTHT |
| | GEKPFACDICGRKFAQSGDLTRHTKIHTHPRAPIPKPFQCRICMRNFSDS |
| | STRKTHIRTHTGEKPFACDICGRKFAQSATRTKHTKIHLRQKDAARGSGG |
| | DAKSLTAWSRTLVTFKDVFVDFTREEWKLLDTAQQIVYRNVMLENYKNLV |
| | SLGYQLTKPDVILRLEKGEEPWLVEREIHQETHPDSETAFEIKSSVRS |
| | (SEQ ID NO: 207) |
| SBS- | MAPKKKRKVGVPAAMAERPFQCRICM [symbol] NFSQSGDLTRHIRTHTGEKPFAC |
| 85001 | DICGRKFAQRSNLNIHTKIHTGSQKPFQCRICM [symbol] NFSRSAHLSRHIRTHT |
| | GEKPFACDICGRKFAQSGDLTRHTKIHTHPRAPIPKPFQCRICMRNFSDS |
| | STRKTHIRTHTGEKPFACDICGRKFAQSATRTKHTKIHLRQKDAARGSGG |
| | DAKSLTAWSRTLVTFKDVFVDFTREEWKLLDTAQQIVYRNVMLENYKNLV |
| | SLGYQLTKPDVILRLEKGEEPWLVEREIHQETHPDSETAFEIKSSVRS |
| | (SEQ ID NO: 208) |
| SBS- | MAPKKKRKVGVPAAMAERPFQCRICM [symbol] NFSMSCCRSAHIRTHTGEKPFAC |
| 85014 | DICGRKFARSADLSRHTKIHTGSQKPFQCRICM [symbol] NFSQSGDLTRHIRTHT |
| | GEKPFACDICGRKFAQRSNLNIHTKIHTGSQKPFQCRICM [symbol] NFSRSAHLS |
| | RHIRTHTGEKPFACDICGRKFAQSGDLTRHTKIHLRQKDAARGSGGDAKS |
| | LTAWSRTLVTFKDVFVDFTREEWKLLDTAQQIVYRNVMLENYKNLVSLGY |
| | QLTKPDVILRLEKGEEPWLVEREIHQETHPDSETAFEIKSSVRS (SEQ |
| | ID NO: 209) |
| SBS- | MAPKKKRKVGVPAAMAERPFQCRICM [symbol] NFSRSDNLARHIRTHTGEKPFAC |
| 85042 | DICGRKFADRSVLHRHTKIHTGSQKPFQCRICMRNFSQSAHRKNHIRTHT |
| | GEKPFACDICGRKFARHSHLTSHTKIHTGSQKPFQCRICMRNFSRSDHLS |
| | EHIRTHTGEKPFACDICGRKFARNDHRTTHTKIHLRQKDAARGSGGDAKS |
| | LTAWSRTLVTFKDVFVDFTREEWKLLDTAQQIVYRNVMLENYKNLVSLGY |
| | QLTKPDVILRLEKGEEPWLVEREIHQETHPDSETAFEIKSSVRS (SEQ |
| | ID NO: 210) |
| SBS- | MAPKKKRKVGVPAAMAERPFQCRICM [symbol] NFSQRNHRTTHIRTHTGEKPFAC |
| 85069 | DICGRKFADRSNLTRHTKIHTGSQKPFQCRICMRNFSMSCCRSAHIRTHT |
| | GEKPFACDICGRKFARSADLSRHTKIHTHPRAPIPKPFQCRICMRNFSRS |
| | DHLSRHIRTHTGEKPFACDICGRKFAQGAHLGAHTKIHLRQKDAARGSGG |
| | DAKSLTAWSRTLVTFKDVFVDFTREEWKLLDTAQQIVYRNVMLENYKNLV |
| | SLGYQLTKPDVILRLEKGEEPWLVEREIHQETHPDSETAFEIKSSVRS |
| | (SEQ ID NO: 211) |
| SBS- | MAPKKKRKVGVPAAMAERPFQCRICM [symbol] NFSRSDALSVHIRTHTGEKPFAC |
| 85096 | DICGRKFADSSHRTRHTKIHTGSQKPFQCRICMRNFSDNRDLSTHIRTHT |
| | GEKPFACDICGRKFADRSNLTRHTKIHTGSQKPFQCRICMRNFSQSGDLT |
| | RHIRTHTGEKPFACDICGRKFATQGYLRKHTKIHLRQKDAARGSGGDAKS |
| | LTAWSRTLVTFKDVFVDFTREEWKLLDTAQQIVYRNVMLENYKNLVSLGY |
| | QLTKPDVILRLEKGEEPWLVEREIHQETHPDSETAFEIKSSVRS (SEQ |
| | ID NO: 212) |
| SBS- | MAPKKKRKVGVPAAMAERPFQCRICM [symbol] NFSRSDHLSQHIRTHTGEKPFAC |
| 85102 | DICGRKFADNSHRTRHTKIHTHPRAPIPKPFQCRICMRNFSDRSDLSRHI |
| | RTHTGEKPFACDICGRKFARSTHLVRHTKIHTGSQKPFQCRICMRNFSDR |
| | SHLTRHIRTHTGEKPFACDICGRKFADRSYRNTHTKIHLRQKDAARGSGG |
| | DAKSLTAWSRTLVTFKDVFVDFTREEWKLLDTAQQIVYRNVMLENYKNLV |
| | SLGYQLTKPDVILRLEKGEEPWLVEREIHQETHPDSETAFEIKSSVRS |
| | (SEQ ID NO: 213) |
| SBS- | MAPKKKRKVGVPAAMAERPFQCRICM [symbol] NFSDRSDLSRHIRTHTGEKPFAC |
| 85105 | DICGRKFARSTHLVRHTKIHTGSQKPFQCRICMRNFSDRSHLTRHIRTHT |
| | GEKPFACDICGRKFADRSYRNTHTKIHTGSQKPFQCRICMRNFSQESDLV |
| | RHIRTHTGEKPFACDICGRKFADRSNLTRHTKIHLRQKDAARGSGGDAKS |
| | LTAWSRTLVTFKDVFVDFTREEWKLLDTAQQIVYRNVMLENYKNLVSLGY |
| | QLTKPDVILRLEKGEEPWLVEREIHQETHPDSETAFEIKSSVRS (SEQ |
| | ID NO: 214) |
| SBS- | MAPKKKRKVGVPAAMAERPFQCRICM [symbol] NFSRSDNLSVHIRTHTGEKPFAC |
| 85177 | DICGRKFARRAHLNQHTKIHTGSQKPFQCRICMRNFSDQSNLRAHIRTHT |
| | GEKPFACDICGRKFARRSDLKRHTKIHTGSQKPFQCRICMRNFSQSGHLA |
| | RHIRTHTGEKPFACDICGRKFAQRVALQAHTKIHLRQKDAARGSGGDAKS |
| | LTAWSRTLVTFKDVFVDFTREEWKLLDTAQQIVYRNVMLENYKNLVSLGY |
| | QLTKPDVILRLEKGEEPWLVEREIHQETHPDSETAFEIKSSVRS (SEQ |
| | ID NO: 215) |
| SBS- | MAPKKKRKVGVPAAMAERPFQCRICM [symbol] NFSQSGNLARHIRTHTGEKPFAC |
| 85180 | DICGRKFAAKWNLDAHTKIHTGSQKPFQCRICMRNFSQSGDLTRHIRTHT |
| | GEKPFACDICGRKFATQGYLRKHTKIHTHPRAPIPKPFQCRICMRNFSRS |
| | DNLSVHIRTHTGEKPFACDICGRKFAQSGDLTRHTKIHLRQKDAARGSGG |
| | DAKSLTAWSRTLVTFKDVFVDFTREEWKLLDTAQQIVYRNVMLENYKNLV |
| | SLGYQLTKPDVILRLEKGEEPWLVEREIHQETHPDSETAFEIKSSVRS |
| | (SEQ ID NO: 216) |
| SBS- | MAPKKKRKVGVPAAMAERPFQCRICM [symbol] NFSTSGNLTRHIRTHTGEKPFAC |
| 85216 | DICGRKFARSELLQSHTKIHTGSQKPFQCRICMRNFSARSTRITHIRTHT |
| | GEKPFACDICGRKFAQSGSLTRHTKIHTHPRAPIPKPFQCRICMRNFSQS |
| | GSLTRHIRTHTGEKPFACDICGRKFAAKWNLDAHTKIHLRQKDAARGSGG |
| | DAKSLTAWSRTLVTFKDVFVDFTREEWKLLDTAQQIVYRNVMLENYKNLV |
| | SLGYQLTKPDVILRLEKGEEPWLVEREIHQETHPDSETAFEIKSSVRS |
| | (SEQ ID NO: 217) |
| SBS- | MAPKKKRKVGVPAAMAERPFQCRICM [symbol] NFSTSGNLTRHIRTHTGEKPFAC |
| 85217 | DICGRKFARSELLQSHTKIHTGSQKPFQCRICM [symbol] NFSARSTRITHIRTHT |
| | GEKPFACDICGRKFAQSGSLTRHTKIHTHPRAPIPKPFQCRICMRNFSQS |
| | GSLTRHIRTHTGEKPFACDICGRKFAAKWNLDAHTKIHLRQKDAARGSGG |
| | DAKSLTAWSRTLVTFKDVFVDFTREEWKLLDTAQQIVYRNVMLENYKNLV |
| | SLGYQLTKPDVILRLEKGEEPWLVEREIHQETHPDSETAFEIKSSVRS |
| | (SEQ ID NO: 218) |
| SBS- | MAPKKKRKVGVPAAMAERPFQCRICM [symbol] NFSTSGNLTRHIRTHTGEKPFAC |
| 85218 | DICGRKFARSELLQSHTKIHTGSQKPFQCRICM [symbol] NFSARSTRITHIRTHT |
| | GEKPFACDICGRKFAQSGSLTRHTKIHTHPRAPIPKPFQCRICM [symbol] NFSQS |
| | GSLTRHIRTHTGEKPFACDICGRKFAAKWNLDAHTKIHLRQKDAARGSGG |
| | DAKSLTAWSRTLVTFKDVFVDFTREEWKLLDTAQQIVYRNVMLENYKNLV |
| | SLGYQLTKPDVILRLEKGEEPWLVEREIHQETHPDSETAFEIKSSVRS |
| | (SEQ ID NO: 219) |
| SBS- | MAPKKKRKVGVPAAMAERPFQCRICM [symbol] NFSRPYTLRLHIRTHTGEKPFAC |
| 85256 | DICGRKFADNSNRIKHTKIHTGSQKPFQCRICM [symbol] NFSRSANLARHIRTHT |
| | GEKPFACDICGRKFATSGHLSRHTKIHTGSQKPFQCRICMRNFSQSGHLA |
| | RHIRTHTGEKPFACDICGRKFARKWTLQGHTKIHLRQKDAARGSGGDAKS |
| | LTAWSRTLVTFKDVFVDFTREEWKLLDTAQQIVYRNVMLENYKNLVSLGY |
| | QLTKPDVILRLEKGEEPWLVEREIHQETHPDSETAFEIKSSVRS (SEQ |
| | ID NO: 220) |
| SBS- | MAPKKKRKVGVPAAMAERPFQCRICM [symbol] NFSRPYTLRLHIRTHTGEKPFAC |
| 85257 | DICGRKFADNSNRIKHTKIHTGSQKPFQCRICM [symbol] NFSRSANLARHIRTHT |
| | GEKPFACDICGRKFATSGHLSRHTKIHTGSQKPFQCRICM [symbol] NFSQSGHLA |
| | RHIRTHTGEKPFACDICGRKFARKWTLQGHTKIHLRQKDAARGSGGDAKS |
| | LTAWSRTLVTFKDVFVDFTREEWKLLDTAQQIVYRNVMLENYKNLVSLGY |
| | QLTKPDVILRLEKGEEPWLVEREIHQETHPDSETAFEIKSSVRS (SEQ |
| | ID NO: 221) |
| SBS- | MAPKKKRKVGVPAAMAERPFQCRICM [symbol] NFSQSADRTKHIRTHTGEKPFAC |
| 85260 | DICGRKFADNSNRIKHTKIHTGSQKPFQCRICM [symbol] NFSRSDNLSTHIRTHT |
| | GEKPFACDICGRKFADSANRIKHTKIHTGSQKPFQCRICM [symbol] NFSRSDHLS |
| | EHIRTHTGEKPFACDICGRKFAQNANRITHTKIHLRQKDAARGSGGDAKS |
| | LTAWSRTLVTFKDVFVDFTREEWKLLDTAQQIVYRNVMLENYKNLVSLGY |
| | QLTKPDVILRLEKGEEPWLVEREIHQETHPDSETAFEIKSSVRS (SEQ |
| | ID NO: 222) |
| SBS- | MAPKKKRKVGVPAAMAERPFQCRICM [symbol] NFSQSADRTKHIRTHTGEKPFAC |
| 85261 | DICGRKFADNSNRIKHTKIHTGSQKPFQCRICMRNFSRSANLSVHIRTHT |
| | GEKPFACDICGRKFADRANLSRHTKIHTHPRAPIPKPFQCRICMRNFSRS |
| | DNLSVHIRTHTGEKPFACDICGRKFAQKATRITHTKIHLRQKDAARGSGG |
| | DAKSLTAWSRTLVTFKDVFVDFTREEWKLLDTAQQIVYRNVMLENYKNLV |
| | SLGYQLTKPDVILRLEKGEEPWLVEREIHQETHPDSETAFEIKSSVRS |
| | (SEQ ID NO: 223) |
| SBS- | MAPKKKRKVGVPAAMAERPFQCRICM [symbol] NFSQSADRTKHIRTHTGEKPFAC |
| 85262 | DICGRKFADNSNRIKHTKIHTGSQKPFQCRICMQNFSRSANLSVHIRTHT |
| | GEKPFACDICGRKFADRANLSRHTKIHTHPRAPIPKPFQCRICMRNFSRS |
| | DNLSVHIRTHTGEKPFACDICGRKFAQKATRITHTKIHLRQKDAARGSGG |
| | DAKSLTAWSRTLVTFKDVFVDFTREEWKLLDTAQQIVYRNVMLENYKNLV |
| | SLGYQLTKPDVILRLEKGEEPWLVEREIHQETHPDSETAFEIKSSVRS |
| | (SEQ ID NO: 224) |
| SBS- | MAPKKKRKVGVPAAMAERPFQCRICM [symbol] NFSQSADRTKHIRTHTGEKPFAC |
| 85263 | DICGRKFADNSNRIKHTKIHTGSQKPFQCRICM [symbol] NFSRSANLSVHIRTHT |
| | GEKPFACDICGRKFADRANLSRHTKIHTHPRAPIPKPFQCRICMQNFSRS |
| | DNLSVHIRTHTGEKPFACDICGRKFAQKATRITHTKIHLRQKDAARGSGG |
| | DAKSLTAWSRTLVTFKDVFVDFTREEWKLLDTAQQIVYRNVMLENYKNLV |
| | SLGYQLTKPDVILRLEKGEEPWLVEREIHQETHPDSETAFEIKSSVRS |
| | (SEQ ID NO: 225) |

1. A fusion protein comprising a zinc finger protein (ZFP) domain and a transcription repressor domain, wherein the ZFP domain binds to a target region of a human alpha-synuclein gene (SNCA gene).

2. The fusion protein of claim 1, wherein the target region is within 1 kb of a transcription start site (TSS) in the SNCA gene.

3. The fusion protein of claim 2, wherein the TSS is TSS1, 2a, or 2b.

4. The fusion protein of claim 3, wherein the target region is within 500 bps upstream of TSS 1, within 500 bps downstream of TSS 1, within 500 bps upstream of TSS 2a, and/or within 500 bps downstream of TSS 2b, of the SNCA gene.

5. The fusion protein of claim 1, wherein the ZFP domain comprises six zinc fingers and the fusion protein represses expression of the SNCA gene by at least about 40%, 75%, 90%, 95%, or 99% with no or minimal detectable off-target binding or activity.

6. The fusion protein of claim 1, wherein the transcription repressor domain comprises a KRAB domain amino acid sequence from human KOX1.

7. The fusion protein of claim 1, wherein the ZFP domain is linked to the transcription repressor domain through a peptide linker.

8. The fusion protein of claim 1, wherein the ZFP domain comprises a DNA-binding recognition helix sequence shown in Table 1.

9. The fusion protein of claim 1, wherein the ZFP domain comprises the DNA-binding recognition helix sequences as shown in a single row of Table 1.

10. A nucleic acid construct comprising a coding sequence for the fusion protein of claim 1, wherein the coding sequence is linked operably to a transcription regulatory element.

11. (canceled)

12. A recombinant virus comprising the nucleic acid construct of claim 10.

13. (canceled)

14. A pharmaceutical composition comprising the recombinant virus of claim 12, and a pharmaceutically acceptable carrier.

15. A host cell comprising the nucleic acid construct of claim 10.

16-17. (canceled)

18. A method of inhibiting expression of alpha-synuclein in a human brain cell, comprising introducing into the cell the fusion protein claim 1.

19. The method of claim 18, wherein the human brain cell is a neuron, a glial cell, an ependymal cell, or a neuroepithelial cell.

20. The method of claim 18, wherein the cell is in the brain of a patient suffering from or at risk of developing Parkinson's disease, Lewy body dementia, Alzheimer's disease, multiple system atrophy, or another synucleinopathy.

21. The method of claim 18, comprising introducing into the cell a recombinant virus that expresses the fusion protein.

22. The method of claim 21, wherein the recombinant virus is an adeno-associated virus (AAV), optionally of serotype 9.

23. A method of treating a synucleinopathy in a patient, comprising administering to the patient a recombinant AAV encoding the fusion protein of of claim 1.

24. The method of claim 23, wherein the AAV is introduced to the patient via intravenous, intrathecal, intracerebroventricular, intra-cisternal magna, intrastriatal, or intranigral injection, or injection into any brain region.

25. The method of claim 23, wherein the synucleinopathy is Parkinson's disease, Lewy body dementia, Alzheimer's disease, or multiple system atrophy.

26-27. (canceled)
